(19) United States
(12) Patent Application Publication
(10) Pub. No.: US 2026/0048100 A1
(43) Pub. Date: Feb. 19, 2026

(54) CYTOKINE FUSION PROTEIN CAPABLE OF CONDITIONAL RELEASE AND ACTIVATION, AND PREPARATION AND USE THEREOF

(71) Applicants: SHANGHAI ZEYIN BIOTECHNOLOGY CO., LTD, Shanghai (CN); SHANGHAI JIAO TONG UNIVERSITY, Shanghai (CN)

(72) Inventors: Huili LU, Shanghai (CN); Wenqiang SHI, Shanghai (CN); Yang WANG, Shanghai (CN); Luyao SONG, Shanghai (CN); Zexin LIU, Shanghai (CN); Qiongya ZENG, Shanghai (CN)

(21) Appl. No.: 19/342,710

(22) Filed: Sep. 29, 2025

Related U.S. Application Data
(63) Continuation of application No. PCT/CN2024/084681, filed on Mar. 29, 2024.

(30) Foreign Application Priority Data
Mar. 30, 2023 (CN) 202310335131.4

(51) Int. Cl. A61K 38/20; A61K 38/17; A61K 39/00; A61K 39/395; A61P 35/00; C07K 14/54; C07K 14/715; C07K 16/22; C07K 16/28

(52) U.S. Cl. CPC A61K 38/2086; A61K 38/1793; A61K 39/3955; A61K 39/39558; A61P 35/00; C07K 14/5443; C07K 14/7155; C07K 16/22; C07K 16/2827; C07K 16/2863; A61K 2039/507; C07K 2317/55; C07K 2317/92; C07K 2319/30; C07K 2319/50

(57) ABSTRACT

Provided are a cytokine fusion protein capable of conditional release and activation, a preparation method therefor, and use thereof. The fusion protein includes: a first structural unit, including at least one of an Fc fragment, an antibody, or an antigen-binding fragment; a second structural unit, including a cleavable peptide linker capable of being cleaved by a specific enzyme in a target tissue and having an amino acid sequence as set forth in any one of SEQ ID No: 29 to SEQ ID No: 32; and a third structural unit, including cytokine or a complex of cytokine and a receptor thereof. The first structural unit is linked to the N- or the C-terminus of the third structural unit via the second structural unit.

### CROSS-REFERENCE TO RELATED APPLICATIONS

This application is a continuation of International Patent Application No. PCT/CN2024/084681 filed on Mar. 29, 2024, which claims priority to and benefits of Chinese patent application No. 202310335131.4, filed with China National Intellectual Property Administration on Mar. 30, 2023, the entire contents of which are incorporated herein by reference.

### STATEMENT REGARDING SEQUENCE LISTING

A Sequence Listing associated with this application is being filed concurrently herewith in ASCII format and is hereby incorporated by reference into the present specification. The text file containing the Sequence listing is titled “Sequence_Listing.xml”, was created on Sep. 18, 2025, and is 62,315 bytes in size.

### FIELD

The present disclosure belongs to the technical field of pharmaceuticals, and specifically relates to a cytokine fusion protein capable of conditional release and activation, a preparation method therefor, and use thereof. More specifically, the present disclosure relates to a fusion protein, a nucleic acid, an expression vector, a host cell, a method for preparing the fusion protein, a fusion protein complex, an immunotherapy cell, a pharmaceutical composition, and use of the fusion protein and the fusion protein complex in the manufacture of a medicament for treating or preventing a cancer, an infectious disease, or an autoimmune disease.

### BACKGROUND

Cytokines play a crucial role in regulating immune responses and serve as the cornerstone of tumor immunotherapy. A variety of cytokines (such as IL-2, IL-7, and IL-15) possess significant anti-tumor effects, but their clinical development has been severely limited by drawbacks such as short half-life, poor targeting, and systemic administration-associated toxic side effects. Currently, various technologies, including Fc fusion, PEG modification, immunocytokines, and conditionally activated cytokine prodrugs, have been developed for the research of next-generation cytokine drugs, aiming to improve the druggability of cytokines. Among these, prodrugs can mask the activity of cytokines in normal tissues, and restore the activity of cytokines through specific protease hydrolysis or other methods after reaching tumor tissues, achieving the goal of targeted anti-tumor therapy. Prodrugs have been extensively studied in the field of antibody drugs, with some products already in the clinical trial stage. However, their application in the field of cytokines is only beginning and represents a frontier of research both domestically and internationally.

Reported conditionally activated cytokine prodrugs, including those based on IL-2, IL-15, IFN-γ, etc., all use affinity peptides or receptors to bind to cytokines. For example, IL-15 prodrugs employ the IL-15Rβ/γ subunits to block binding to IL-15Rβ/γ on target cells, thereby masking its function. However, this affinity-masking approach presents potential obstacles in drug development. For example, after the affinity peptides or receptors are cleaved in the tumor microenvironment, they may still retain the ability to bind to the cytokine in their free states, which interferes with the cytokine's recognition of target cells and their activity, thereby affecting the anti-tumor effect. In addition, incorporating extra affinity-masking peptides or receptors in prodrug molecules increases the molecular weight, immunogenicity, structural complexity, and the like. Therefore, there is an urgent need to develop new masking technologies for current conditionally activated cytokine prodrugs.

Immunocytokines are bifunctional fusion proteins constructed by combining monoclonal antibodies with cytokines. They can utilize the targeting ability of antibodies to enrich cytokines at tumor sites, enhancing the targeting of cytokines. Also, they can exert synergistic anti-tumor effects of both antibodies and cytokines. Based on the remarkable efficacy and favorable safety shown in preclinical studies, pharmaceutical companies both domestically and internationally are advancing research products into clinical trials. The most advanced molecule is L19-TNF, which is jointly developed by the University of Zurich in Switzerland and Philogen. It has entered Phase III clinical trials when used in combination with doxorubicin for the treatment of metastatic soft tissue sarcomas. However, studies have found that the proportion of immunocytokines targeted to tumors is generally less than 0.1%, and their side effect levels are similar to those of the cytokine alone. As a result, the dosages of immunocytokine drugs in the current clinical trial stage are limited to relatively low levels. For example, the Phase III clinical dosage of L19-TNF is only 13 μg/kg, which is far below the typical dose range between 10 mg/kg and 20 mg/kg for conventional monoclonal antibodies. Therefore, safety remains one of the major bottlenecks limiting the clinical application of immunocytokines. Structural optimization of immunocytokines to further enhance their targeting and safety is the key to advancing such molecules into clinical practice.

### SUMMARY

In view of the above-described deficiencies or limitations in the related art, the present disclosure seeks to provide a cytokine fusion protein capable of masking and regulating cytokine activity through steric hindrance, and further capable of fusing with antibody-based molecules to generate immunocytokine prodrugs, thereby improving the therapeutic targeting and efficacy of such drugs.

In an aspect of the present disclosure, the present disclosure provides a fusion protein. The cytokine fusion protein includes a first structural unit, a second structural unit, and a third structural unit. The first structural unit includes at least one of an Fc fragment, an antibody, or an antigen-binding fragment thereof. The second structural unit includes a cleavable peptide linker having an amino acid sequence as set forth in any one of SEQ ID No: 29 to SEQ ID No: 32. The third structural unit includes cytokine or a complex of cytokine and a receptor thereof. The first structural unit is linked to the N- or C-terminus of the third structural unit via the second structural unit. Steric hindrance of the first structural unit can mask the structure of the third structural unit, thereby regulating binding of the third structural unit to its target cell and altering its activity. The second structural unit contains a cleavable peptide linker (also referred to herein as a cleavable linker), which can be cleaved by a target tissue (such as a protease overexpressed in a tumor site, optionally a matrix metalloproteinase, a serine protease, or an asparagine endopeptidase). Experiments disclosed herein have shown that the third structural unit of the fusion protein of the present disclosure exhibits different activities before and after cleavage, resulting in better targeting and safety.

In some embodiments of the present disclosure, the first structural unit of the fusion protein of the present disclosure is linked to the N-terminus of the third structural unit via the second structural unit. As an example, where the first structural unit includes an Fc fragment and the third structural unit is an active complex of cytokine IL-15 and a receptor thereof, the fusion protein has the structure illustrated in FIG. 1a, including sequentially from the N-terminus to the C-terminus: an antibody Fc fragment, a cleavable linker, IL-15, and the sushi domain of IL-15Ra. The present disclosure has found that, compared with a fully active Fc-sushi-IL-15 superagonist protein (fusion protein LH02 in the Examples), the cell proliferation-promoting activity of IL-15 in the fusion protein is significantly reduced before cleavage of the second structural unit, while greatly enhanced after cleavage, thereby achieving significantly improved safety in mice.

In some embodiments of the present disclosure, the first structural unit of the fusion protein of the present disclosure is linked to the C-terminus of the third structural unit via the second structural unit. As an example, where the first structural unit includes an antibody Fc fragment and the third structural unit is a complex of IL-15 and the sushi domain of IL-15Rα, the fusion protein (named LIC18) has the structure illustrated in FIG. 1f, including sequentially from the N-terminus to the C-terminus: the sushi domain of IL-15Rα, IL-15, the second structural unit containing the cleavable linker, and the antibody Fc fragment. The sushi domain is linked to IL-15 by a flexible linker. The present disclosure has found that, compared with the fusion protein where the sushi-IL-15 complex is located at the C-terminus of Fc, the fusion protein where the sushi-IL-15 complex is located at the N-terminus of Fc exhibits significantly reduced activity before cleavage of the second structural unit, while greatly enhanced after proteolytic cleavage of the second structural unit, thereby achieving improved targeting and safety.

In some embodiments of the present disclosure, the first structural unit is an antibody Fab, whose heavy chain and light chain are respectively linked to the N- or C-terminus of the third structural unit via the second structural unit.

In some embodiments of the present disclosure, the first structural unit is an antibody Fab, and one of the heavy chain and light chain is linked to the N- or C-terminus of the third structural unit via the second structural unit. As an example, where the first structural unit is a monoclonal antibody Fab fragment targeting PD-L1, the second structural unit is a cleavable linker containing a urokinase substrate, and the third structural unit is a complex of cytokine IL-15 and an IL-15Rα fragment containing the sushi domain, the third structural unit is fused to the C-terminus of the heavy chain of the first structural unit via the second structural unit, and the resulting immunocytokine (named LIC110) has the structure illustrated in FIG. 1c, with the light chain being the light chain of an anti-PD-L1 monoclonal antibody, and the heavy chain including sequentially from the N-terminus to the C-terminus: the heavy chain variable region and CHI region of the anti-PD-L1 antibody, the cleavable linker, IL-15, and the IL-15Rα fragment containing the sushi domain. The present disclosure has found that the steric hindrance of Fab of the LIC110 fusion protein masks the activity of IL-15 before enzymatic cleavage, and after enzymatic cleavage, the portion of IL-15 and the IL-15Rα fragment containing the sushi domain (referred to as ILR) is released, thereby removing the steric hindrance and restoring the activity.

In some embodiments of the present disclosure, the third structural unit of the fusion protein of the present disclosure includes IL-15 and the IL-15Rα fragment containing the sushi domain. The sushi domain is a fragment of 65 to 85 amino acids in length, preferably 85 amino acids. IL-15 is linked to the IL-15Rα fragment by a flexible linker to form an IL-15 superagonist (referred to as ILR). Compared with monomeric IL-15, the IL-15 superagonist exhibits better in vivo stability and half-life, stronger ability to activate T cells and NK cells at the tumor site, and better therapeutic efficacy against tumors.

In another aspect of the present disclosure, the present disclosure provides a nucleic acid encoding the above-described fusion protein.

In some embodiments of the present disclosure, the nucleic acid molecule is DNA.

It should be noted that, for the nucleic acid mentioned in the present disclosure, those skilled in the art should understand that they actually include any one or both of the complementary double strands. In the present disclosure, for convenience, only one strand is given in most cases, but the other strand complementary to the one strand is actually also disclosed. In addition, the nucleic acid sequences of the present disclosure include a DNA form or an RNA form, the disclosure of one of which implies the disclosure of the other.

In another aspect of the present disclosure, the present disclosure provides an expression vector, which carries the above-described nucleic acid. When the above-described nucleic acid is connected to the vector, the nucleic acid can be directly or indirectly connected to the control elements on the vector, as long as these control elements can control the translation and expression and the like of the nucleic acid. These control elements may be directly derived from the expression vector itself, or they may be exogenous, i.e., derived from the expression vector itself. The nucleic acid is operably connected to the control elements.

As used herein, “operably connected” means that an exogenous gene is connected to the vector, in such a manner that the control elements in the vector, such as transcription control sequences and translation control sequences, can play their expected functions of regulating the transcription and translation of the exogenous gene. Common vectors may be, for example, plasmids, bacteriophages, etc. Once the expression vector according to some specific embodiments of the present disclosure is introduced into suitable recipient cells, the expression of the above-described fusion protein can be effectively achieved under the mediation of the regulatory system, thereby obtaining a large amount of the fusion protein in vitro.

In some embodiments of the present disclosure, the expression vector is a eukaryotic expression vector or a prokaryotic expression vector. Preferably, the expression vector is a plasmid expression vector.

In another aspect of the present disclosure, the present disclosure provides a recombinant cell, which carries the above-described nucleic acid or the above-described expression vector or expresses the above-described fusion protein. Under suitable conditions, the recombinant cell can be used to effectively express the above-described fusion protein.

It should be noted that, the “suitable conditions” described in the present disclosure refer to conditions suitable for the expression of the fusion protein described in the present disclosure. Those skilled in the art can understand that the conditions suitable for the expression of the fusion protein include, but are not limited to, appropriate transformation or transfection methods, appropriate transformation or transfection conditions, healthy host cell states, appropriate host cell density, suitable cell culture environment, and suitable cell culture time. The “suitable conditions” are not particularly limited, and those skilled in the art can optimize the most suitable conditions for the expression of the fusion protein according to the specific environment of the laboratory.

In some embodiments of the present disclosure, the recombinant cell is obtained by introducing the above-described expression vector into a host cell.

In some embodiments of the present disclosure, the recombinant cell is a eukaryotic cell.

In some embodiments of the present disclosure, the recombinant cell is a mammalian cell.

In some embodiments of the present disclosure, the present disclosure provides a fusion protein complex. As an optional embodiment, the fusion protein complex of the present disclosure is a homodimeric or heterodimeric protein containing the fusion protein of the present disclosure. Fusion proteins in the complex can be linked to each other by an interchain bond formed between Fc regions.

In another aspect of the present disclosure, the present disclosure provides an immunocytokine including the above-described fusion protein. The fusion protein includes an antibody or antigen-binding fragment. The antibody or antigen-binding fragment targets a tumor antigen or an immune checkpoint.

In some embodiments of the present disclosure, the target tumor antigen or immune checkpoint includes at least one of EGFR, VEGF, Claudin 18.2, Nectin-4, GPC-3, PD-L1, PD-1, TIGIT, LAG3, TIM-3, and CTLA-4.

In some embodiments of the present disclosure, the antibody or antigen-binding fragment is from a human IgG1 or IgG4 antibody.

In some embodiments of the present disclosure, as an example, where the third structural unit is a complex of cytokine IL-15 and an IL-15Rα fragment containing the sushi domain (such as ILR in FIG. 1), its N-terminus is fused with a monoclonal antibody sequence targeting PD-L1, and the resulting immunocytokine (named LH05) has the structure illustrated in FIG. 1n, including sequentially from the N-terminus to the C-terminus: a Fab fragment of the anti-PD-L1 antibody, the Fc fragment, the cleavable linker, IL-15, and the IL-15Rα fragment containing the sushi domain. The present disclosure has found that, the immunocytokine is capable of binding to the PD-L1 antigen protein. In addition, compared with the fully active Fc-sushi-IL-15 superagonist protein, the cell proliferation-promoting activity of IL-15 in the immunocytokine is significantly reduced before cleavage of the second structural unit, while greatly enhanced after cleavage, thereby achieving significantly improved targeting and safety in mice.

In another aspect of the present disclosure, the present disclosure provides an immunotherapy cell, which expresses the above-described fusion protein. As can be seen above, the fusion protein is capable of masking and regulating cytokine activity through steric hindrance, and further capable of fusing with antibody-based molecules to generate immunocytokine prodrugs, thereby improving the therapeutic targeting and efficacy of such drugs. As a result, the immune cell can express the above-described fusion protein and can be used to treat an infectious disease, a cancer, or an autoimmune disease.

In another aspect of the present disclosure, the present disclosure provides a pharmaceutical composition. The pharmaceutical composition includes the above-described fusion protein, the above-described nucleic acid, the above-described expression vector, the above-described recombinant cell, the above-described fusion protein complex, the above-described immunocytokine, or the above-described immunotherapy cell. The pharmaceutical composition of the present disclosure can be used to treat an infectious disease, a cancer, or an autoimmune disease.

In another aspect of the present disclosure, the present disclosure provides a combination agent or kit. The combination agent or kit includes the above-described fusion protein or the above-described fusion protein complex as a first active ingredient; and a monoclonal antibody targeting a tumor as a second active ingredient. The technical solution of combining the fusion protein of the present disclosure with a monoclonal antibody drug targeting a tumor, for example, combination of the fusion protein of the present disclosure with an anti-VEGF antibody or an anti-Her2 antibody, has exerted a synergistic anti-tumor effect in a mouse tumor model.

In some embodiments of the present disclosure, the monoclonal antibody targeting the tumor includes an anti-VEGF antibody and/or an anti-Her2 antibody.

In another aspect of the present disclosure, the present disclosure provides use of the above-described fusion protein, fusion protein complex, immunotherapy cell, pharmaceutical composition, or combination agent or kit in the manufacture of a medicament for treating an infectious disease, a cancer, or an autoimmune disease.

In another aspect of the present disclosure, the present disclosure provides a method for treating a cancer, an infectious disease, or an autoimmune disease. The method includes administering to a subject a pharmaceutically acceptable amount of the above-described fusion protein, the above-described fusion protein complex, the above-described immunocytokine, the above-described immunotherapy cell, the above-described pharmaceutical composition, or the above-described combination agent or kit.

In some embodiments of the present disclosure, the cancer includes, but is not limited to, leukemia (e.g., acute leukemia, acute lymphocytic leukemia, acute myeloid leukemia, acute myeloblastic leukemia, acute promyelocytic leukemia, acute myelomonocytic leukemia, acute monocytic leukemia, acute erythrocytic leukemia, chronic leukemia, chronic myeloid leukemia, or chronic lymphocytic leukemia), polycythemia vera, lymphoma (Hodgkin's disease, non-Hodgkin's disease), macroglobulinemia, and solid tumors such as sarcomas and malignant tumors (e.g., fibrosarcoma, myxosarcoma, liposarcoma, chondrosarcoma, osteosarcoma, chordoma, angiosarcoma, endotheliosarcoma, lymphangiosarcoma, lymphangioendotheliosarcoma, synovioma, mesothelioma, Ewen's tumor, leiomyosarcoma, rhabdomyosarcoma, colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, liver cancer, cholangiocarcinoma, choriocarcinoma, seminoma, embryonal carcinoma, Wilms' tumor, cervical cancer, uterine cancer, testicular cancer, lung cancer, small cell lung cancer, bladder cancer, epithelial carcinoma, glioma, astrocytoma, medulloblastoma, craniopharyngioma, ependymoma, pinealma, hemangioblastoma, acoustic neuroma, oligodendroglioma, schwannoma, meningioma, melanoma, neuroblastoma, and retinoblastoma). The infectious disease includes, but is not limited to, smallpox virus infection, HIV infection, bacterial infection, fungal infection, and HBV infection. The autoimmune disease includes, but is not limited to, multiple sclerosis, psoriasis, rheumatoid arthritis, systemic lupus erythematosus, ankylosing spondylitis, Crohn's disease, gastritis, and mucositis.

### BRIEF DESCRIPTION OF THE DRAWINGS

Other features, objects and advantages of the present disclosure will become more apparent upon reading the detailed description of non-limiting embodiments with reference to the following drawings:

FIG. 1 is a schematic diagram illustrating the structures of the fusion proteins of the present disclosure, in which: a represents Fc-L-C where Fc serves as the first structural unit; b represents a fusion protein Fab-L-C where Fab serves as the first structural unit; c represents Fab-L-c where Fab serves as the first structural unit and only one cytokine is fused to the constant region of the heavy chain or light chain; d represents scFv-L-C where scFv serves as the first structural unit; e represents scFv-Fc-L-C in a form of scFv fusion; f represents C-L-Fc where Fc serves as the first structural unit and is fused to the C-terminus of the third structural unit; g represents C-L-Fab where Fab serves as the first structural unit; i represents C-L-Fab where Fab serves as the first structural unit and only one cytokine is fused to the constant region of the heavy chain or light chain; j represents C-L-scFv where scFv serves as the first structural unit; k represents C-L-Fc in the form of scFv fusion; 1 represents a protein complex/immunocytokine formed by fusion of Fc with Fab-L-C where Fab serves as the first structural unit and only one cytokine is fused to the constant region of the heavy chain or light chain; m represents a protein complex/immunocytokine formed by fusion of Fc with C-L-Fab where Fab serves as the first structural unit and only one cytokine is fused to the constant region of the heavy chain or light chain; and n represents a protein complex/immunocytokine formed by fusion of Fab with Fc-L-C; in which: L represents a cleavable linker, and C represents a cytokine or a complex of a cytokine and a receptor thereof.

FIG. 2 is a diagram showing protein A affinity chromatography purification of the LIC11 fusion protein in Example 1 of the present disclosure.

FIG. 3 is an SDS-PAGE electrophoretogram of the purified LIC11 fusion protein in Example 1 of the present disclosure, in which: NR represents a non-reduced sample, R represents a reduced sample, S represents the sample before loading, FL represents flow-through liquid during protein A affinity chromatography, E represents an eluted LIC11 fusion protein, and NaOH represents impurities eluted with a NaOH solution after chromatography.

FIG. 4 shows Western blot identification of the LIC11 fusion protein in Example 1 of the present disclosure, in which Non-reduced represents a non-reduced sample, and Reduced represents a reduced sample.

FIG. 5 is an SDS-PAGE diagram for detecting cleavage efficiency of the LIC11 fusion protein by uPA enzyme at different time points in Example 2 of the present disclosure.

FIG. 6 shows the detection results of Mo7e cell proliferation activity induced by the LIC11 fusion protein before and after cleavage by uPA enzyme in Example 3 of the present disclosure.

FIG. 7 is a diagram showing protein A affinity chromatography purification of the LIC15 fusion protein in Example 4 of the present disclosure.

FIG. 8 is an SDS-PAGE electrophoretogram of the purified LIC15 fusion protein in Example 1 of the present disclosure, in which: NR represents a non-reduced sample, R represents a reduced sample, S represents the sample before loading, FL represents flow-through liquid during protein A affinity chromatography, E represents an eluted LIC15 fusion protein, and NaOH represents impurities eluted with a NaOH solution after chromatography.

FIG. 9 is an SDS-PAGE diagram for detecting cleavage efficiency of the LIC15 fusion protein by MMP-2 enzyme in Example 5 of the present disclosure.

FIG. 10 shows the detection results of enzymatic cleavage and release of the LIC15 fusion protein after masking the activity of IL-15 in Example 5 of the present disclosure.

FIG. 11 is an SDS-PAGE electrophoretogram of the purified LIC12 fusion protein in Example 6 of the present disclosure, in which: NR represents a non-reduced sample, R represents a reduced sample. S represents the sample before loading, FL represents flow-through liquid during protein A affinity chromatography, E represents an eluted LIC12 fusion protein, and NaOH represents impurities eluted with a NaOH solution after chromatography.

FIG. 12 is an SDS-PAGE electrophoretogram of the purified LIC13 fusion protein in Example 6 of the present disclosure, in which: NR represents a non-reduced sample, R represents a reduced sample, S represents the sample before loading, FL represents flow-through liquid during protein A affinity chromatography, E represents an eluted LIC13 fusion protein, and NaOH represents impurities eluted with a NaOH solution after chromatography.

FIG. 13 is an SDS-PAGE electrophoretogram of the purified LIC14 fusion protein in Example 6 of the present disclosure, in which: NR represents a non-reduced sample, R represents a reduced sample, S represents the sample before loading, FL represents flow-through liquid during protein A affinity chromatography, E represents an eluted LIC14 fusion protein, and NaOH represents impurities eluted with a NaOH solution after chromatography.

FIG. 14 is an SDS-PAGE diagram showing the enzymatic cleavage efficiency of the fusion proteins corresponding to different linkers in Example 7 of the present disclosure.

FIG. 15 shows the results of Mo7e cell proliferation activity induced by the fusion proteins corresponding to different linkers in Example 7 of the present disclosure.

FIG. 16 is a curve showing tumor growth of mice in the experimental group and the negative control group in Example 7 of the present disclosure.

FIG. 17 is a curve showing tumor growth of mice in the LIC11 fusion protein group and the positive control group in Example 7 of the present disclosure.

FIG. 18 shows body weights of mice in the LIC11 fusion protein group and the positive control group in Example 7 of the present disclosure.

FIG. 19 is a graph showing results of body weight changes in mice after in vivo administration of the LIC11 fusion protein in Example 8 of the present disclosure.

FIG. 20 shows spleen images and spleen weight of mice after in vivo administration of the LIC11 fusion protein in Example 8 of the present disclosure.

FIG. 21 is a graph showing results of CD8⁺ T cell counts in the peripheral blood of mice after in vivo administration of the LIC11 fusion protein in Example 8 of the present disclosure.

FIG. 22 is a graph showing results of NK cell counts in the peripheral blood of mice after in vivo administration of the LIC11 fusion protein in Example 8 of the present disclosure.

FIG. 23 is a diagram showing protein L affinity chromatography purification of the LIC110 fusion protein in Example 9 of the present disclosure.

FIG. 24 is an SDS-PAGE electrophoretogram of the purified LIC110 fusion protein in Example 9 of the present disclosure, in which: S represents the sample before loading, FL represents flow-through liquid during protein A affinity chromatography, and E represents an eluted LIC110 fusion protein.

FIG. 25 shows the detection results of Mo7e cell proliferation activity induced by the LIC110 fusion protein before and after cleavage by uPA enzyme in Example 10 of the present disclosure.

FIG. 26 is a diagram showing protein A affinity chromatography purification of the LIC18 fusion protein in Example 11 of the present disclosure.

FIG. 27 is an SDS-PAGE electrophoretogram of the purified LIC18 fusion protein in Example 11 of the present disclosure, in which: NR represents a non-reduced sample, S represents the sample before loading, FL represents flow-through liquid, E1 represents the elution peak 1, E2 represents the elution peak 2, and NaOH represents impurities eluted with a NaOH solution.

FIG. 28 shows the detection results of Mo7e cell proliferation activity induced by the LIC18 fusion protein before and after cleavage by uPA enzyme in Example 12 of the present disclosure.

FIG. 29 is a diagram showing protein A affinity chromatography purification of the LH05 fusion protein in Example 13 of the present disclosure.

FIG. 30 is an SDS-PAGE electrophoretogram of the purified LH05 fusion protein in Example 13 of the present disclosure, in which: NR represents a non-reduced sample, R represents a reduced sample, S represents the sample before loading, FL represents flow-through liquid, E represents the eluate, and NaOH represents impurities eluted with a NaOH solution.

FIG. 31 shows Western blot identification of the LH05 fusion protein (anti-IgG(H+L) antibody) in Example 13 of the present disclosure, in which: NR represents a non-reduced sample, R represents a reduced sample, S represents the sample before loading, FL represents flow-through liquid, E represents the eluate, and NaOH represents impurities eluted with a NaOH solution.

FIG. 32 shows Western blot identification of the LH05 fusion protein (anti-IL-15 antibody) in Example 13 of the present disclosure.

FIG. 33 shows the detection results of affinity of the LH05 fusion protein to the human PD-L1 antigen in Example 14 of the present disclosure.

FIG. 34 shows the detection results of affinity of the LH05 fusion protein to the mouse PD-L1 antigen in Example 14 of the present disclosure.

FIG. 35 is an SDS-PAGE diagram of the LH05 fusion protein cleaved by uPA enzyme in Example 15 of the present disclosure.

FIG. 36 shows the detection results of Mo7e cell proliferation activity induced by the LH05 fusion protein before and after cleavage by uPA enzyme in Example 16 of the present disclosure.

FIG. 37 shows the detection results of toxicity assessment of the LH05 fusion protein in mice after in vivo administration in Example 17 of the present disclosure, in which: A is a curve showing body weight changes, and B is a curve showing survival rates.

FIG. 38 shows the detection results of safety assessment of the LH05 fusion protein in mice after in vivo administration in Example 17 of the present disclosure, in which: A represents spleen weight; B represents CD8⁺ T cell counts and NK cell counts in peripheral blood; and C represents the levels of inflammatory factors IFN-γ and IL-6 in the plasma.

FIG. 39 is a curve showing tumor growth of mouse RM-1 tumors treated with the LH05 fusion protein in Example 18 of the present disclosure.

FIG. 40 is a curve showing survival rate of mice with RM-1 tumors treated with the LH05 fusion protein in Example 18 of the present disclosure.

FIG. 41 is a curve showing tumor growth of mouse HT-29 xenograft tumors treated with the LH05 fusion protein in combination with an anti-VEGF monoclonal antibody in Example 19 of the present disclosure.

FIG. 42 is an SDS-PAGE diagram of the purified LIC23 fusion protein in Example 20 of the present disclosure, in which: NR represents a non-reduced sample, R represents a reduced sample, S₀ represents the sample before loading, FL represents flow-through liquid, E represents the eluate, and NaOH represents impurities eluted with a NaOH solution.

FIG. 43 is an SDS-PAGE diagram of the LIC23 fusion protein cleaved by MMP enzyme in Example 21 of the present disclosure.

FIG. 44 shows the detection results of Mo7e cell proliferation activity induced by the LIC23 fusion protein before and after cleavage by MMP enzyme in Example 22 of the present disclosure.

FIG. 45 is a curve showing tumor growth of mouse U87 xenograft tumors treated with the LIC31 fusion protein in Example 23 of the present disclosure.

FIG. 46 is a diagram showing protein A affinity chromatography purification of the LIC20 fusion protein in Example 24 of the present disclosure.

FIG. 47. is an SDS-PAGE electrophoretogram of the purified LIC20 fusion protein in Example 24 of the present disclosure, in which: NR represents a non-reduced sample, R represents a reduced sample, Sup represents the supernatant of the medium, FL represents flow-through liquid during protein A affinity chromatography, E represents the eluted LIC20 fusion protein, and NaOH represents impurities eluted with a NaOH solution after chromatography.

FIG. 48 shows the detection results of Mo7e cell proliferation activity induced by the LIC20 fusion protein before and after cleavage by uPA enzyme in Example 25 of the present disclosure, in which the dotted line represents LIC20, and the solid line represents the LIC11 control.

FIG. 49 shows the detection results of Mo7e cell proliferation activity induced by the LIC19 fusion protein before and after cleavage by uPA enzyme in Example 26 of the present disclosure, in which the dotted line represents LIC19, and the solid line represents the LIC11 control.

FIG. 50 shows the detection results of Mo7e cell proliferation activity induced by the LIC20 fusion protein before and after cleavage by uPA enzyme in Example 25 of the present disclosure, in which the dotted line represents LIC111, and the solid line represents the LIC11 control.

### DETAILED DESCRIPTION OF THE EMBODIMENTS

The present disclosure will be described in detail below with reference to the accompanying embodiments. It should be understood that, the specific embodiments described herein are only used to explain the present disclosure rather than to limit the present disclosure.

It should be noted that, the embodiments of the present disclosure and features in the embodiments can be combined with each other where there is no conflict.

It should be noted that, in the present disclosure, endpoints and any value of the ranges shall not be limited to the exact range or value, and those ranges or values should be understood to include values close to those ranges or values. For numerical ranges, endpoints of respective ranges, an endpoint and individual point value of respective ranges, and individual point values can be combined with each other to obtain one or more new numerical ranges, which should be deemed to be specifically disclosed herein. If the specific technologies or conditions are not specified in the examples, they shall be carried out according to the technologies or conditions as described in the literature in the art or according to the product instructions. The reagents and instruments used without indicating the manufacturer are all conventional products that can be purchased commercially.

It should be noted that, unless otherwise indicated, scientific and technical terms used herein have the meanings commonly understood by those skilled in the art. In addition, the laboratory operating procedures for cell culture, molecular genetics, nucleic acid chemistry, and immunology used herein are conventional procedures widely used in the corresponding fields.

The present disclosure provides a fusion protein including a first structural unit, a second structural unit, and a third structural unit. The first structural unit includes at least one of an Fc fragment, an antibody, or an antigen-binding fragment. The second structural unit includes a cleavable peptide linker having an amino acid sequence as set forth in any one of SEQ ID No: 29 to SEQ ID No: 32. The third structural unit includes cytokine or a complex of cytokine and a receptor thereof. The first structural unit is linked to the N-terminus or the C-terminus of the third structural unit via the second structural unit.

In the fusion protein of the present disclosure, the steric hindrance of the first structural unit and the second structural unit can mask the structure of the third structural unit, thereby regulating binding of the third structural unit to a target cell thereof and altering activity of the third structural unit. In addition, after the cleavable peptide linker is cleaved by a specific enzyme in a target tissue, the third structural unit can be separated from the first structural unit, endowing the third structural unit with greater freedom and facilitating exertion of its activity.

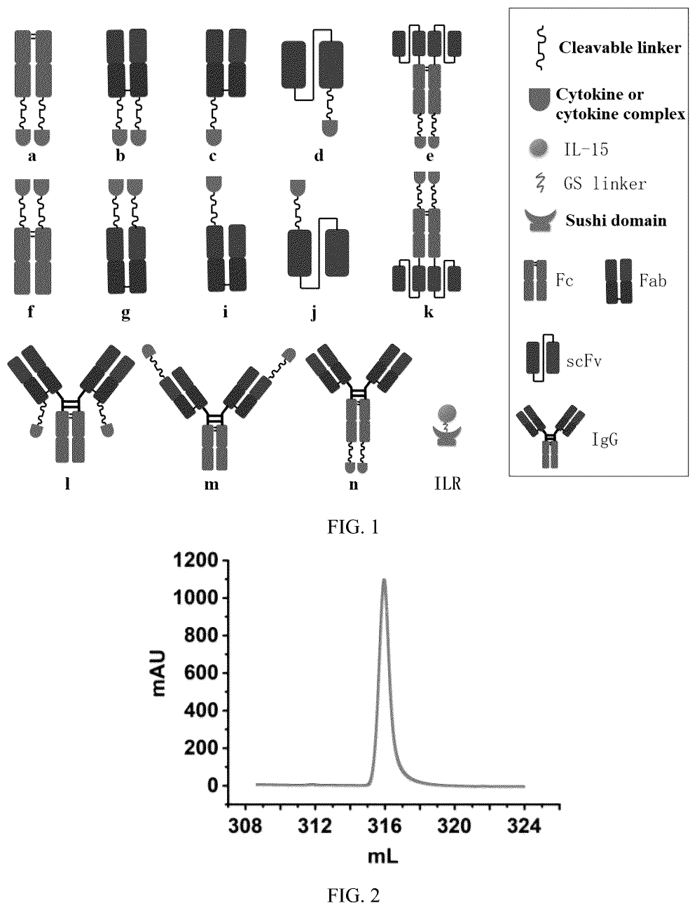

That is, the connection order of domains of the fusion protein in the present disclosure, from the N-terminus to the C-terminus, is any one selected from the following:
1) an antibody Fc fragment, a cleavable linker, cytokine or a complex of cytokine and a receptor thereof (as illustrated in a of FIG. 1);
2) cytokine or a complex of cytokine and a receptor thereof, a cleavable linker, and an antibody Fc fragment (as illustrated in f of FIG. 1).

The fusion protein with the structure of 1) or 2) above can be further fused with an antigen-binding fragment (e.g., Fab, scFv) to form a new fusion protein (as illustrated in e, k, and n of FIG. 1).
3) an antibody or antigen-binding fragment, a cleavable linker, cytokine or a complex of cytokine and a receptor thereof (as illustrated in b, c, and d of FIG. 1);
4) cytokine or a complex of cytokine and a receptor thereof, a cleavable linker, and an antibody or antigen-binding fragment thereof (as illustrated in g, i, and j of FIG. 1);

The fusion protein with the structure of 3) or 4) above can be further fused with an antibody Fc fragment to form a new fusion protein (as illustrated in 1 and m of FIG. 1). The two peptides of the antibody Fc fragment can be both fused with a fusion protein with the above-described structure simultaneously, or one of the two peptides can be fused with the fusion protein with the above-described structure. The specific type is not limited and falls within the protection scope of the present disclosure.

The third structural unit is affected by the steric hindrance of the first structural unit and the second structural unit, such that, in the fused state, its binding to the receptor is restricted, resulting in relatively low biological activity. After the cleavable linker of the second structural unit is cleaved, the third structural unit is released, thereby restoring its affinity for the receptor and recovering its biological activity to a high level. As a result, the toxic and side effects on non-target tissues are reduced while the therapeutic effect on the target is enhanced.

In some embodiments, the second structural unit of the present disclosure is a cleavable linker. Preferably, the cleavable linker is a peptide linker that can be cleaved by the target tissue, such as a protease overexpressed in the tumor tissue. After the fusion protein of the present disclosure reaches the target tissue, such as the tumor tissue, the linker is cleaved by the protease overexpressed in the target tissue, releasing the cytokine or the complex of cytokine and the receptor thereof of the third structural unit.

In some embodiments of the present disclosure, the first structural unit is located at the N-terminus of the fusion protein and includes an immunoglobulin molecule or an immunoglobulin Fc fragment, as illustrated in e, 1, and n of FIG. 1.

In some embodiments of the present disclosure, the first structural unit is located at the C-terminus of the fusion protein and includes an immunoglobulin molecule or an immunoglobulin Fc fragment, as illustrated in k and m of FIG. 1.

In an alternative embodiment, the first structural unit is linked to the N-terminus of the third structural unit via the second structural unit. As an example, where the first structural unit includes an Fc fragment and the third structural unit is IL-15 and an IL-15R fragment containing the sushi domain, the fusion protein includes sequentially from the N-terminus to the C-terminus: an Fc fragment, a cleavable linker, IL-15, and the IL-15R fragment containing the sushi domain (hereinafter referred to as LIC11, as illustrated in a of FIG. 1). The present disclosure has found that, Fc and the linker form steric hindrance to the binding region of IL-15 and a receptor thereof, thereby affecting the activity of IL-15. As a result, the activity of LIC11 is masked before cleavage and is relatively low. However, after cleavage, IL-15 is released, the steric hindrance is eliminated, and the activity is restored. Consequently, compared with the IL-15 monomer, LIC11 exhibits significantly enhanced tumor targeting and safety.

In another alternative embodiment, the first structural unit is linked to the N-terminus of the third structural unit via the second structural unit. As an example, where the first structural unit is an antibody Fab fragment, and the third structural unit is IL-15 and the IL-15R fragment containing the sushi domain, the C-terminus of the light chain and heavy chain of Fab is respectively fused sequentially with the cleavable linker, IL-15, and the IL-15R fragment containing the sushi domain (hereinafter referred to as LIC19, as illustrated in b of FIG. 1). The present disclosure has found that, Fab and the linker form steric hindrance to the binding region of IL-15 and a receptor thereof, thereby affecting the activity of IL-15. As a result, the activity of LIC19 is masked before cleavage and is relatively low. However, after cleavage, IL-15 is released, the steric hindrance is eliminated, and the activity is restored. Consequently, compared with the IL-15 monomer, LIC19 exhibits significantly enhanced tumor targeting and safety.

In an alternative embodiment, the first structural unit is linked to the N-terminus of the third structural unit via the second structural unit. As an example, where the first structural unit is an antibody Fab fragment, and the third structural unit is IL-15 and the IL-15R fragment containing the sushi domain, the C-terminus of the light chain or heavy chain of Fab is sequentially fused with the cleavable linker, IL-15, and the IL-15R fragment containing the sushi domain (hereinafter referred to as LIC110, as illustrated in c of FIG. 1). The present disclosure has found that, Fab and the linker form steric hindrance to the binding region of IL-15 and the receptor thereof, thereby affecting the activity of IL-15. As a result, the activity of LIC110 is masked before cleavage and is relatively low. However, after cleavage, IL-15 is released, the steric hindrance is eliminated, and the activity is restored. Consequently, compared with the IL-15 monomer, LIC110 exhibits significantly enhanced tumor targeting and safety.

In another alternative embodiment, the first structural unit is linked to the N-terminus of the third structural unit via the second structural unit. As an example, where the first structural unit is an antibody scFv, the third structural unit is IL-15 and the IL-15R fragment containing the sushi domain, the fusion protein includes sequentially from the N-terminus to the C-terminus: scFv, the cleavable linker, IL-15, and the IL-15R fragment containing the sushi domain (hereinafter referred to as LIC111, as illustrated in e of FIG. 1). The present disclosure has found that, Fab and the linker form steric hindrance to the binding region of IL-15 and a receptor thereof, thereby affecting the activity of IL-15. As a result, the activity of LIC111 is masked before cleavage and is relatively low. However, after cleavage, IL-15 is released, the steric hindrance is eliminated, and the activity is restored. Consequently, compared with the IL-15 monomer, LIC111 exhibits significantly enhanced tumor targeting and safety.

In another embodiments of the present disclosure, the first structural unit is located at the C-terminus of the fusion protein and includes an antibody Fc fragment.

In some embodiments of the present disclosure, the antibody or antigen-binding fragment can be selected from antibodies or antibody fragments targeting tumor antigens or immune checkpoints, generating a new immunocytokine. These antigens and immune checkpoints include at least one of EGFR, VEGF, Claudin 18.2, Nectin-4, GPC-3, PD-L1, PD-1, TIGIT, LAG3, TIM-3, and CTLA-4.

In the present disclosure, “EGFR” refers to an epidermal growth factor receptor, whose mutation or overexpression generally causes tumor. After dimerization, EGFR activates downstream signal pathways in cells, which are associated with proliferation of tumor cells, angiogenesis, tumor invasion, metastasis, and apoptosis inhibition. The antibody or antigen-binding fragment that binds to EGFR can inhibit the activity of EGFR and prevent its phosphorylation, thereby inhibiting activation of downstream signal pathways and further achieving anti-tumor effects.

In the present disclosure, “VEGF” refers to a vascular endothelial growth factor, which acts on endothelial cells through a paracrine mechanism and plays an important role in promoting angiogenesis, inhibiting endothelial cell apoptosis, and increasing vascular permeability. VEGF is overexpressed in nearly all human tumors and tumor cell lines. The antibody or antigen-binding fragment that binds to VEGF can inhibit binding of VEGF to its receptors VEGFR-1 and VEGFR-2 on endothelial cells, thereby inactivating VEGF and reducing tumor angiogenesis, and thus inhibiting tumor growth.

In the present disclosure, “Claudin 18.2” refers to the 18.2 isomer molecule of the Claudin family of tight junction proteins and is a highly specific cell surface molecule. It is only expressed on differentiated gastric mucosal epithelial cells in normal tissues and is highly expressed in solid tumors such as gastric cancer, pancreatic cancer, ovarian cancer, cholangiocarcinoma, and lung adenocarcinoma. The antibody that binds to Claudin 18.2 can specifically recognize tumor cells and trigger antibody-dependent cellular cytotoxicity (ADCC), complement-dependent cytotoxicity (CDC), apoptosis, and inhibition of cell proliferation, possessing a strong ability to eliminate cancer cells and to control disease.

In the present disclosure, “PD-L1” refers to programmed death-ligand 1 and is an immune checkpoint protein. Tumor cells overexpress PD-L1, which continuously activates the PD-1/PD-L1 signal pathway and causes various types of immune suppression. The antibody or antigen-binding fragment that binds to PD-L1, as a blocking agent of the immune checkpoint protein, can inhibit the PD-1/PD-L1 pathway, block the co-inhibitory function of CD80 and PD-L1, and facilitate the comprehensive activation of T-cell functions and the promotion of cytokine production.

In the present disclosure, “PD-1” refers to a programmed death protein 1 and a co-inhibitory receptor that can be induced on the surfaces of T cells, B cells, monocytes, and natural killer cells.

In the present disclosure, “TIGIT” refers to a protein containing T cell immunoglobulin and ITIM domain. Abnormal expression of TIGIT can be detected in a variety of tumors, which can lead to immune cell dysfunction and is associated with tumor progression and poor prognosis. Blocking TIGIT can reverse immune cell exhaustion and exert anti-tumor effects, making it a new-generation target for immunotherapy.

In the present disclosure, “LAG3” refers to lymphocyte activation gene-3, “TIM-3” refers to T cell immunoglobulin mucin molecule 3, and “CTLA-4” refers to cytotoxic T lymphocyte-associated antigen-4. All three are immunosuppressive receptors and participate in the immune regulation of T cells.

In the fusion protein according to the above-described embodiments of the present disclosure, the first structural unit, the second structural unit, and the third structural unit all adopt a monomeric form, which exhibits a half-life and targeting property similar to those of a dimeric form. In another embodiments of the present disclosure, at least one of the first structural unit, the second structural unit, and the third structural unit is in the dimeric form.

In some embodiments of the present disclosure, the second structural unit of the present disclosure includes a peptide linker cleavable by a matrix metalloproteinase. All the peptide linkers cleavable by the matrix metalloproteinase that are known in the art, such as those disclosed in WO2019010219A, can be used in the present disclosure, and the above-described document is incorporated herein by reference. In some embodiments, the amino acid sequence of the second structural unit includes SGQLLGFLTA, which is a substrate for the matrix metalloproteinases MMP-2/9/14.

In some embodiments of the present disclosure, the target tissue includes a protease; optionally, the protease is selected from proteases overexpressed in the tumor tissue. In some preferred embodiments of the present disclosure, the protease is a matrix metalloproteinase, a serine protease, or an asparagine endopeptidase.

Exemplarily, the serine protease is selected from urokinase and trypsin. All the peptide linkers cleavable by the serine protease that are known in the art can be used in the present disclosure. In some embodiments, the cleavable peptide linker includes LSGRSDNH, which is a substrate for the urokinase.

In some embodiments of the present disclosure, the cleavable peptide linker includes a peptide linker cleavable by the asparagine endopeptidase. All the peptide linkers cleavable by the asparagine endopeptidase that are known in the art can be used in the present disclosure. In some embodiments, the amino acid sequence of the second structural unit includes AANL, which is a substrate for the asparagine endopeptidase.

In some embodiments of the present disclosure, the cleavable peptide linker includes a flexible peptide segment and/or a protease substrate sequence.

In some embodiments of the present disclosure, an amino acid sequence of the flexible peptide segment includes at least one selected from (GS)n, (GGS)n, (GGSG)n, (GSSG)n, (GGGS)n, (GGGGS)n, and (GSGGS)n, where n is any integer between 1 and 20.

Exemplarily, n may be 1, 2, 3, 4, 5, 6, 7, 8, 9, 10, 11, 12, 13, 14, 15, 16, 17, 18, 19, or 20.

In some embodiments of the present disclosure, an amino acid sequence of the flexible peptide segment is 1 to 30 amino acids in length.

In some embodiments of the present disclosure, the cleavable peptide linker includes a flexible peptide segment and a protease substrate sequence.

In some embodiments of the present disclosure, the cleavable peptide linker is selected from a flexible peptide segment-protease substrate sequence and a flexible peptide segment-protease substrate sequence-flexible peptide segment.

In some embodiments of the present disclosure, the flexible peptide segment located between the protease substrate sequence and the third structural unit is 5 to 13 amino acids in length. The present disclosure has unexpectedly discovered that, compared with a case where there is no flexible peptide segment between the enzymatic cleavage substrate sequence and IL-15, the fusion protein exhibits better enzymatic cleavage efficiency when the flexible peptide segment between the enzymatic cleavage substrate sequence and IL-15 is 5 to 13 amino acids in length.

In some embodiments of the present disclosure, the cleavable peptide linker has an amino acid sequence as set forth in any one of SEQ ID No: 29 to SEQ ID No: 32. The present disclosure has found that, the cleavable peptide linker in the fusion protein can influence the strength of the steric masking effect of the first structural unit on the third structural unit and the cleavage efficiency.

In some embodiments of the present disclosure, the cleavable peptide linker has an amino acid sequence as set forth in SEQ ID No: 29.

In some embodiments of the present disclosure, the complex of cytokine and the receptor thereof is a complex of IL-15 and a receptor thereof with a sushi domain. In some embodiments of the present disclosure, the complex of IL-15 and the receptor thereof with the sushi domain includes a fusion protein of IL-15-flexible linker-sushi domain. That is, in the complex of IL-15 and a receptor thereof with the sushi domain of the present disclosure, IL-15 is linked to the sushi domain of IL-15Rα via the flexible linker to form an IL-15 superagonist. Compared with monomeric IL-15, the IL-15 superagonist exhibits better in vivo stability and half-life and can reduce difficulties and improve efficiency in terms of production processes and quality control. The flexible linker includes repeats of sequences selected from GS, GSGGS, GGGGS and/or GGGS. In some embodiments of the present disclosure, an amino acid sequence of the flexible linker is set forth in SEQ ID No: 34.

In some embodiments of the present disclosure, the flexible linker includes at least one selected from (GS)n, (GSGGS)n, (GGGGS)n, and/or (GGGS)n, where n is any integer between 1 and 20.

Exemplarily, n may be 1, 2, 3, 4, 5, 6, 7, 8, 9, 10, 11, 12, 13, 14, 15, 16, 17, 18, 19, or 20.

In the present disclosure, the “cytokine” refers to a class of low-molecular-weight proteins with a wide range of biological activities, including families such as interleukins, interferons, tumor necrosis factors, hematopoietic factors, growth factors, and chemokines. The cytokines exert their biological effects by binding to corresponding cytokine receptors on the cell surface. Therefore, the fusion protein of the present disclosure masks the receptor-binding region of the cytokine through the steric hindrance between the first structural unit and the second structural unit and interferes with its binding to the receptor, thereby affecting its biological activity. After enzymatic cleavage of the second structural unit, the cytokine is partially released, thereby restoring its ability to bind to the receptor on the cell surface and recovering its activity. In the present disclosure, “fusion protein” refers to a biologically active polypeptide and an effector molecule that are linked (i.e., fused) by a genetic recombination method, a chemical method, or other appropriate methods. If necessary, the fusion molecule can be fused at one or more positions via an amino acid linker. The fusion protein can exist in the form of a monomer or a polymer (e.g., a dimer). In some embodiments of the present disclosure, “IL-15” refers to interleukin 15, including any natural (wild-type) IL-15, functional fragments or mutants thereof, preferably mammalian interleukin 15, and more preferably human interleukin 15. In some embodiments, mammalian interleukin 15 includes, but is not limited to, interleukin 15 derived from mice/rats, pigs, rabbits, sheep/goats, monkeys, and cats. In some embodiments of the present disclosure, the IL-15 is a human IL-15 molecule or a mutant of the human IL-15 molecule.

In some embodiments, an amino acid sequence of the human IL-15 molecule is set forth in SEQ ID No: 1. In some embodiments, the mutant of the human IL-15 molecule is obtained by one or more amino acid substitutions, additions, and/or deletions of a native human IL-15 molecule, such as a truncated human IL-15 polypeptide sequence. In some embodiments, the mutant of the human IL-15 molecule is a mutant having one or more amino acid substitutions selected from C42S, L45C, Q48C, V49C, L52C, E53C, E87C, and E89C. In some embodiments, the mutant of the human IL-15 molecule is a mutant having one or more amino acid substitutions selected from N1D, N4D, D8N, D30N, D61N, E64Q, N65D, and Q108E. For example, in some embodiments, the amino acid substitution is Q108E. In some embodiments, the amino acid substitution is N65D. In some embodiments, the amino acid substitution is N1D/N65D. In some embodiments, the amino acid substitution is N4D/N65D. In some embodiments, the amino acid substitution is D30N/E64Q/N65D. Preferably, the mutant of the human IL-15 molecule is a mutant having N72D mutation. The present disclosure has found that, the fusion protein containing the IL-15 mutant having N72D mutation of the present disclosure exhibits enhanced biological activity. Preferably, an amino acid sequence of IL-15 of the present disclosure is set forth in SEQ ID No: 4.

In the present disclosure, the “sushi domain of IL-15Rα” refers to a functional fragment within the extracellular domain of the IL-15α receptor, with 65 to 85 amino acids in length. It may be a functional fragment or mutant of IL-15Rα from any species, preferably human IL-15Rα. In some embodiments, the IL-15Rα of the present disclosure has an amino acid sequence as set forth in NCBI Reference Sequence No. NP_002180.1. In another embodiments, an extracellular domain fragment of IL-15Rα containing the sushi domain is used, and an amino acid sequence thereof is set forth in SEQ ID No: 5.

In the present disclosure, the “antibody” refers to an immunoglobulin molecule typically composed of two pairs of polypeptide chains, each pair consisting of a light chain (L chain) and a heavy chain (H chain). In a general sense, the heavy chain can be understood as the polypeptide chain with a larger molecular weight in an antibody, while a light chain refers to the polypeptide chain with a smaller molecular weight in an antibody. Each heavy chain is composed of a heavy chain variable region (VH) and a heavy chain constant region (CH), and the heavy chain constant region consists of three domains (CH-1, CH-2, and CH-3). Each light chain is composed of a light chain variable region (VL) and a light chain constant region (CL), and the light chain constant region consists of one domain CL. The VH and VL regions can be further subdivided into regions with high variability (referred to as complementary determining regions (CDRs)), and the variable regions (VH and VL) of heavy chain/light chain pairs form antibody binding sites. The term “antibody” is not limited by any specific method for preparing antibodies. For example, the antibody includes, in particular, recombinant antibodies, monoclonal antibodies and polyclonal antibodies. Antibodies may be antibodies of different types, such as IgG or a mutant thereof, IgA1, IgA2, IgD, IgE, or IgM antibody. In some embodiments of the present disclosure, the antibody that binds to a tumor antigen or an immune checkpoint is a full-length immunoglobulin, including IgG1, IgG2, IgG3, and IgG4.

In the present disclosure, the “antigen-binding fragment” refers to a polypeptide including a portion or all of a full-length antibody. It lacks at least some amino acids present in the full-length chain but still retains the ability to specifically bind to the same antigen that the full-length antibody binds to, and/or competes with the full-length antibody for specific binding to the antigen. It is also referred to as an antigen-binding portion. For example, this fragment may include a portion or all of the CDRs of the antibody. Such fragments are biologically active because they bind to the antigen and can compete with other antigen-binding molecules (including intact antibodies) for binding to a given epitope. Antigen-binding fragments of antibodies can be prepared by recombinant DNA technology or by enzymatic or chemical cleavage of intact antibodies. In some embodiments of the present disclosure, the antibody includes at least one selected from a polyclonal antibody, a full-length monoclonal antibody, a Fab antibody, a Fab′ antibody, a F(ab′)₂ antibody, a Fv antibody, a single-chain antibody, a single-domain antibody, and a minimum recognition unit; or the antigen-binding fragment includes at least one selected from a F(ab′)₂ fragment, a Fab′ fragment, a Fab fragment, a F(ab)₂ fragment, a Fv fragment, a scFv fragment, a scFv-Fc fusion protein, a scFv-Fv fusion protein, and a minimum recognition unit.

In some preferred embodiments of the present disclosure, the antigen-binding fragment of the antibody is Fab, Fab′, (Fab′)₂, Fv, scFv, or VHH, or a fusion form of Fab, Fab′, (Fab′)₂, Fv, scFv, or VHH with scFv.

In the present disclosure, “Fab” is a fragment obtained by treating an antibody molecule with papain (which cleaves the amino acid residue at position 224 of the H chain). Approximately half of the N-terminus side of the H chain is linked to the entire L chain via a disulfide bond.

In the present disclosure, “F(ab′)₂” is an antibody fragment obtained by treating the portion below the two disulfide bonds in the hinge region of an antibody molecule with pepsin. It has a molecular weight of approximately 100,000 Da and contains two Fab regions linked at the hinge position.

In the present disclosure, “Fab′” is an antibody fragment obtained by cleaving the disulfide bonds in the hinge region of the above-described F(ab′)₂. Fab′ can be prepared by treating F(ab′)₂, which specifically recognizes and binds to an antigen, with a reducing agent such as dithiothreitol.

In the present disclosure, “Fv” refers to the smallest functional fragment of an antibody molecule that retains the antigen-binding site and consists of a light chain variable region and a heavy chain variable region, which are linked together via non-covalent bonds.

In the present disclosure, “scFv” refers to an antibody fragment that includes the heavy chain variable region (VH) and light chain variable region (VL) of an antibody. The size of scFv is generally ⅙ that of a complete antibody, and it is preferably an amino acid chain sequence encoded by a single nucleotide chain.

In the present disclosure, “VHH” refers to a single variable region of a heavy chain antibody and possesses antigen-binding capacity.

In the present disclosure, the “antibody Fc fragment” refers to a crystallizable segment, which corresponds to the CH-2 and CH-3 domains of an antibody molecule. In some embodiments, the antibody Fc fragment is derived from human IgG1 or IgG4 and includes one or more amino acid substitutions, additions, or deletions compared to the wild type, such that the fusion protein weakens or even eliminates ADCC and/or CDC functions, reducing nonspecific immune responses.

In some embodiments of the present disclosure, the N-terminus of the first structural unit is further fused with a Fab targeting PD-L1, resulting in a fusion protein with PD-L1 targeting capability. In an embodiment, the third structural unit is IL-15 and the sushi domain of IL-15Rα, and the new fusion protein obtained is an anti-PD-L1/IL-15 immunocytokine, designated as LH05 (as illustrated in “n” of FIG. 1). An amino acid sequence of the LH05 light chain is set forth in SEQ ID No: 2, and an amino acid sequence of the heavy chain is set forth in SEQ ID No: 6. In an embodiment, the nucleotide sequences encoding the light and heavy chains of LH05 are set forth in SEQ ID No: 7 and SEQ ID No: 8, respectively.

In another embodiments, the third structural unit is linked to the N-terminus of the third structural unit via a cleavable second structural unit. Alternatively, the third structural unit is linked to the C-terminus of the third structural unit via a cleavable second structural unit. Exemplarily, the third structural unit is linked to the C-terminus of the light chain of the human IgG1 immunoglobulin Fab that binds to PD-L1 via a cleavable second structural unit. In this embodiment, the light chain of the fusion protein includes from the N-terminus to the C-terminus: the light chain of the human IgG1 immunoglobulin that binds to PD-L1, a cleavable linker, IL-15 and the sushi domain of IL-15Rα. The heavy chain of the fusion protein is the heavy chain of the human IgG1 immunoglobulin that binds to PD-L1 (as illustrated in 1 of FIG. 1).

In some embodiments of the present disclosure, the fusion protein has an amino acid sequence as set forth in any one of SEQ ID No: 11, SEQ ID No: 14, SEQ ID No: 16, SEQ ID No: 20, SEQ ID No: 21, and SEQ ID No: 22; or
the fusion protein includes a first peptide segment with an amino acid sequence as set forth in SEQ ID No: 6 and a second peptide segment with an amino acid sequence as set forth in SEQ ID No: 2; or
the fusion protein includes a first peptide segment with an amino acid sequence as set forth in SEQ ID No: 24 and a second peptide segment with an amino acid sequence as set forth in SEQ ID No: 2; or
the fusion protein includes a first peptide segment with an amino acid sequence as set forth in SEQ ID No: 25 and a second peptide segment with an amino acid sequence as set forth in SEQ ID No: 26; or
the fusion protein includes a first peptide segment with an amino acid sequence as set forth in SEQ ID No: 27 and a second peptide segment with an amino acid sequence as set forth in SEQ ID No: 28.

An embodiment of the present disclosure relates to a fusion protein complex. The fusion protein complex of the present disclosure may be a dimeric protein formed by the binding of two different or identical fusion proteins. That is, the fusion protein complex may be a homodimer or a heterodimer. The fusion proteins in the fusion protein complex can be linked to each other via interchain bonds formed between Fc fragments and are preferably covalently linked via disulfide bonds formed between the Fc fragments to form a dimer. In some embodiments, the fusion protein complex includes two fusion proteins of the present disclosure.

The present disclosure further provides the nucleic acid encoding the fusion protein, the corresponding expression vector, and the recombinant cell.

The nucleic acid of the present disclosure may be DNA or RNA molecules, or nucleic acid analogs. The nucleic acid molecule of the present disclosure may include naturally occurring or artificially generated nucleic acid residues. The nucleic acid molecule of the present disclosure may be single-stranded or double-stranded, linear or circular, natural or synthetic, and unless otherwise specified, there is no restriction on its size. The nucleic acid molecule may also include a promoter, which may be homologous or heterologous.

The nucleic acid molecule of the present disclosure can be cloned into a vector. The “vector” in the present disclosure includes plasmids, cosmids, viruses, phages, and other vectors commonly used in genetic engineering. In some embodiments, these vectors are suitable for transforming cells, eukaryotic cells such as fungal cells, and microbial cells such as yeast or prokaryotic cells. In a preferred embodiment, these vectors are suitable for stable transformation of bacterial cells, for example, to transcribe the nucleic acid molecules of the present disclosure.

The vector of the present disclosure may be an expression vector. Suitable eukaryotic expression vectors, which have been extensively described in the literature, can all be used in the present disclosure. In an embodiment, the expression vector may contain a marker gene, an origin of replication that ensures replication in the selected host, a promoter, and a transcription termination signal. Between the promoter and the termination signal, there is preferably at least one restriction site into which the nucleic acid sequence/molecule to be expressed can be inserted. Preferably, the expression vector of the present disclosure is selected from pET series expression vectors, pGEX series expression vectors, pcDNA series expression vectors, and pCMV series expression vectors. More preferably, the expression vector of the present disclosure is the pMF09 expression vector.

In an embodiment, the fusion protein is expressed in the recombinant cell, and the antibody is subsequently isolated and typically purified to a pharmaceutically acceptable purity. For protein expression, the nucleic acid encoding the protein is inserted into an expression vector using standard methods. Expression is carried out in suitable stable recombinant cells, and the protein is recovered from the cells (supernatant or lysed cells).

In another embodiment, the nucleic acid molecule of the present disclosure and/or the vector containing the nucleic acid molecule of the present disclosure can be transduced, transformed, transfected, or introduced into the host cell by other means. For example, the host cell is a eukaryotic or prokaryotic cell, preferably a eukaryotic cell. As a non-limiting example, the host cell is a mammalian cell. The host cell of the present disclosure may be human, yeast, or fungal cells, such as Chinese hamster ovary (CHO) cells, baby hamster kidney (BHK, ATCC CCL 10) cells, Sertoli cells from baby mice, monkey kidney cells (COS cells), monkey kidney CVI cells transformed by SV40 (COS-7, ATCC CRL 1651), human embryonic kidney (HEK-293) cells, monkey kidney (CVI, ATCC CCL-70) cells, African green monkey kidney (VERO-76, ATCC CRL-1587) cells, human cervical cancer (HELA, ATCC CCL-2) cells, etc. Preferably, the host cell of the present disclosure is a human HEK293 cell.

The present disclosure further provides a method for preparing the above-described fusion protein. The method includes: culturing the recombinant cell of the present disclosure under conditions suitable for expression of the fusion protein, and recovering the fusion protein from the cell or cell culture supernatant. In an embodiment, the method for preparing the fusion protein of the present disclosure includes: constructing an expression vector containing a gene encoding the fusion protein, constructing the recombinant cell containing the expression vector through transiently transfection of the recombinant cell, culturing the recombinant cell, collecting the cell supernatant, and purifying the fusion protein by affinity chromatography using Protein A/G as a filler.

Suitable conditions for expressing the above-described fusion protein should be known to those skilled in the art. Those skilled in the art can select an applicable culture medium based on experience to culture the cells under conditions suitable for the growth of the recombinant cells. After the recombinant cell grows to an appropriate cell density, the selected promoter is induced using a suitable method (such as temperature shift or chemical induction), and then the cells are cultured for an additional period of time. The recombinant polypeptide in the above method can be expressed intracellularly, on the cell membrane, or secreted extracellularly.

Methods for isolating and purifying the fusion protein are well known to those skilled in the art. Examples of these methods include, but are not limited to, conventional renaturation, treatment with protein precipitants, centrifugation, osmotic shock, ultra-treatment, ultracentrifugation, molecular sieve chromatography, adsorption chromatography, ion exchange chromatography, high-performance liquid chromatography, and various other liquid chromatography techniques, as well as combinations of these methods.

The present disclosure further provides a pharmaceutical composition containing the fusion protein, the nucleic acid, the expression vector, the recombinant cell, and/or the fusion protein complex of the present disclosure.

In some embodiments, the pharmaceutical composition of the present disclosure further includes a monoclonal antibody targeting a tumor antigen or an immune checkpoint, such as PD-1, PD-L1, LAG3, TIM-3, CTLA-4, Her2, EGFR, Claudin 18.2, VEGF, Claudin 18.2, Nectin-4, GPC-3, VEGF, CD20, and CD33. Combined use with the fusion protein of the present disclosure can exert a synergistic anti-tumor effect and enhance the therapeutic efficacy on diseases.

In some embodiments, the pharmaceutical composition of the present disclosure may further include a pharmaceutically acceptable carrier. The pharmaceutically acceptable carrier refers to a conventional pharmaceutical carrier in the field of pharmacy, such as diluents, excipients, and water; a filler such as starch, sucrose, lactose, and microcrystalline cellulose; a binder such as cellulose derivatives, alginates, gelatin, and polyvinylpyrrolidone; a wetting agent such as glycerol; a disintegrant such as carboxymethyl starch sodium, hydroxypropyl cellulose, cross-linked carboxymethyl cellulose, agar, calcium carbonate, and sodium bicarbonate; an absorption enhancer such as quaternary ammonium compounds; a surfactant such as cetyl alcohol and sodium lauryl sulfate; an adsorption carrier such as kaolin and bentonite; or a lubricant such as talc, calcium stearate, magnesium stearate, silicon dioxide, and polyethylene glycol. In addition, other adjuvants such as flavoring agents and sweeteners can also be added to the composition.

Examples of suitable pharmaceutical carriers are well known in the art. The pharmaceutical compositions containing such carriers can be formulated by well-known conventional methods. In some embodiments, the pharmaceutical composition of the present disclosure may further contain other active ingredients for treatment.

The pharmaceutical composition of the present disclosure can be administered in different ways, such as enteral, oral (e.g., pills, tablets, buccal, sublingual, disintegrants, capsules, films, liquid solutions or suspensions, powders, and solid crystals or liquids), rectal (e.g., suppositories and enemas), via injection (e.g., intravenous, subcutaneous, intramuscular, intraperitoneal, and intradermal), via inhalation (e.g., intrabronchial), topical, vaginal, transdermal, or intranasal administration. Preferably, the pharmaceutical composition of the present disclosure is in the form of a lyophilized formulation or an aqueous solution. The clinical dosage regimen is determined by the attending physician and clinical factors. As is well known in the medical field, the dosage for any individual patient depends on many factors, including the patient's physical build, body surface area, age, drug to be administered, gender, time and route of administration, general health, and other drugs administered simultaneously. The pharmaceutical composition of the present disclosure can be administered locally or systemically. Preferably, it can be administered intravenously or subcutaneously. The pharmaceutical composition of the present disclosure can also be administered directly to the target site, for example, through targeted drug delivery to internal or external target sites.

The present disclosure further provides use of the fusion protein and the fusion protein complex described in the present disclosure in the manufacture of a medicament for treating a cancer, an infectious disease, or an autoimmune disease. The cancer includes but is not limited to leukemia (e.g., acute leukemia, acute lymphocytic leukemia, acute myeloid leukemia, acute myeloblastic leukemia, acute promyelocytic leukemia, acute myelomonocytic leukemia, acute monocytic leukemia, acute erythrocytic leukemia, chronic leukemia, chronic myeloid leukemia, or chronic lymphocytic leukemia), polycythemia vera, lymphoma (Hodgkin's disease, non-Hodgkin's disease), macroglobulinemia, and solid tumors such as sarcomas and malignant tumors (e.g., fibrosarcoma, myxosarcoma, liposarcoma, chondrosarcoma, osteosarcoma, chordoma, angiosarcoma, endotheliosarcoma, lymphangiosarcoma, lymphangioendotheliosarcoma, synovioma, mesothelioma, Ewen's tumor, leiomyosarcoma, rhabdomyosarcoma, colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, liver cancer, cholangiocarcinoma, choriocarcinoma, seminoma, embryonal carcinoma, Wilms' tumor, cervical cancer, uterine cancer, testicular cancer, lung cancer, small cell lung cancer, bladder cancer, epithelial carcinoma, glioma, astrocytoma, medulloblastoma, craniopharyngioma, ependymoma, pinealma, hemangioblastoma, acoustic neuroma, oligodendroglioma, schwannoma, meningioma, melanoma, neuroblastoma, and retinoblastoma). The infectious disease includes, but is not limited to, smallpox virus infection, HIV infection, bacterial infection, fungal infection, and HBV infection. The autoimmune disease includes, but is not limited to, multiple sclerosis, psoriasis, rheumatoid arthritis, systemic lupus erythematosus, ankylosing spondylitis, Crohn's disease, gastritis, and mucositis.

In the present disclosure, the above-described medicament includes therapeutic cells, such as CAR T cells and CAR NK cells. The fusion protein of the present disclosure is expressed or modified on the surface of the cells, which helps enhance the survival and activity of cells at the tumor site, thereby improving the efficacy of cell therapy for diseases.

In the present disclosure, “treatment” or “treat” refers to obtaining a desired pharmacological and/or physiological effect. The effect may be prophylactic in terms of complete or partial prevention of the disease or its symptoms, and/or therapeutic in terms of partial or complete cure of the disease and/or adverse effects caused by the disease. As used herein, the term “treatment” or “treat” is intend to aim at diseases in mammals, especially humans, including: inhibiting diseases, for example, retarding the progression of the diseases; or alleviating diseases, such as mitigating the symptoms associated with the diseases. As used herein, the term “treatment” or “treat” includes treating, curing, alleviating, ameliorating, mitigating or inhibiting disease in an individual by administrating any of a medicament or a compound to the individual, including but not limited to, administering the medicament described herein to an individual in need thereof.

Additional aspects and advantages of the present disclosure will be provided at least in part in the following description, or will become apparent at least in part from the following description, or can be learned from practicing of the present disclosure.

### Example 1: Preparation of LIC11 Fusion Protein Capable of Cleavage and Release of IL-15

### 1. Construction of Expression Vector Encoding LIC11 Fusion Protein

The nucleotide sequence as set forth in SEQ ID No: 13 was prepared by gene synthesis and PCR technology, and this nucleotide sequence encoded the LIC11 fusion protein with the amino acid sequence as set forth in SEQ ID No: 14. As illustrated in FIG. 1a, the Fc fragment in the LIC11 fusion protein was located at the N-terminus and linked to the IL-15 complex via a cleavable linker (i.e., a cleavable peptide linker). The IL-15 complex contained a fusion protein of IL-15 (an amino acid sequence as set forth in SEQ ID No: 1) and the IL-15Rα sushi domain (an amino acid sequence as set forth in SEQ ID No: 5) linked via a flexible linker (an amino acid sequence as set forth in SEQ ID No: 34).

The obtained fragment was inserted between the Hind III and Not I restriction sites of the pMF09 plasmid through homologous recombination. The resulting homologous recombination product was transformed into the DH5a strain to obtain the expression vector pMF09/LIC11. Strains with correctly constructed vectors were verified by sequencing. After culturing to the logarithmic growth phase, glycerol with a final concentration of 20% was added for long-term storage at −80° C.

### 2. Plasmid Amplification

Strains containing the expression vector pMF09/LIC11 were cultured in an expanded volume at an inoculum ratio of 1:100. Plasmids were extracted by the alkaline lysis method using the E.Z.N.A. Endo-free Plasmid Maxi Kit (catalog number D6926-03) provided by OMEGA, filtered through a sterile filter membrane, and then stored at −20° C.

### 3. Cell Culture and Transient Transfection for Expression of LIC11 Fusion Protein

### 1) Cell Preparation

Human HEK293 cells were cultured in suspension. The formula of the medium was prepared by mixing two media SFM4HEK293 Medium (Hyclone, USA) and Freestyle 293 Expression Medium (Invitrogen, USA) at a volume ratio of 1:1, followed by the addition of fetal bovine serum with a final concentration of 2%. The suspension was passaged into the fresh medium at a density of 1×10⁶ cells/mL and cultured in suspension in a constant temperature shaker (5% CO₂) at 37° C. with a rotational speed of 125 rpm.

One day before transfection, cells were passaged into a 500 ml conical culture flask, and the cell state and density were adjusted to 4×10⁶ cells/mL with a culture volume of 200 mL.

On the day of transfection, when the cell density reached 6×10⁶ cells/mL and the cell viability exceeded 95%, the cells were subjected to transfection. 100 mL of cell culture was subjected to centrifugation to obtain cells. The cells were washed once with Freestyle 293 medium, and then suspended in 100 mL of Freestyle 293 medium.

### 2) Plasmid Dilution

The plasmid was used at a mass of 0.5 μg plasmid per 10⁶ cells, diluted to 40

| | |
|---|---|
| Mass, μg | 300 |
| Concentration, ng/μL | 780 |
| Dilute to 40 ng/μL, Volume/mL | 385 μL →7.5 mL |

3) Plasmid Encapsulation with PEI

Plasmid and PEI (polyethylenimine, transfection reagent, purchased from Polysciences) were mixed at a mass ratio of 1:5. Specifically, 1.5 mL of PEI was added to the diluted plasmid, mixed thoroughly, and allowed to stand for 10 to 15 minutes for encapsulation. The time was controlled as the mixture became turbid.

### 4) PEI Transfection

The PEI-encapsulated plasmid was added to the cells and placed in a shaker for culture.

### 5) Medium Supplementation at 4 h

After 4 hours, an equal volume of 100 mL of SFM4 HEK293 medium, 1 mL of VPA (200×, 750 mmol/L, final concentration of 3.75 mmol/L), and 400 μL of G418 (50 mg/mL) were added.

### 6) Medium Supplementation at 24 h

200 μL of anti-clumping solution (1000×) and 5.0 mL of 20% TN1 (final concentration of 0.5%) were added.

### 7) Cell Viability was Monitored.

After 6 days to 7 days of culture, when the cell viability dropped below 50%, the cell culture supernatant was collected for subsequent purification step.

### 4. Purification of LIC11 Fusion Protein

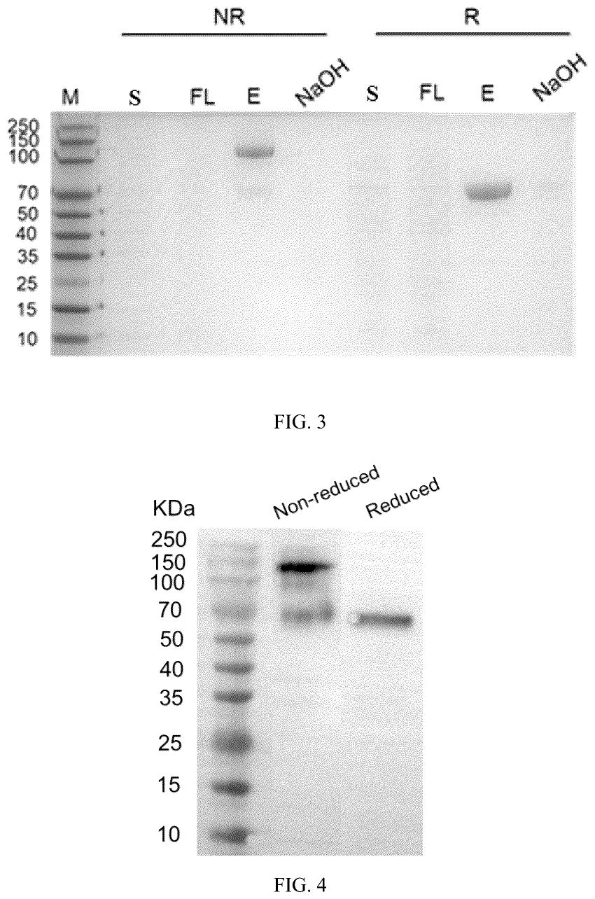

1) Sample Preparation: The cell suspension was centrifuged at 7000 rpm for 30 minutes. The precipitate was discarded, and the supernatant was filtered through a 0.45 μm filter membrane for later use.
2) The protein purification system was rinsed with 20% ethanol. The Protein A column was rinsed and equilibrated with 10 column volumes of double-distilled water and loading buffer, respectively.
3) The sample was loaded using a peristaltic pump of the protein purifier. The flow rate was set according to the column volume, generally not exceeding one column volume per minute, and the flow-through was collected.
4) After loading, the Protein A column was re-equilibrated with 10 column volumes of loading buffer.
5) Elution was performed using an elution buffer (citric acid-sodium citrate buffer, pH=2.8). The eluate was collected in fractions, with 1 mL per tube. The elution peak was monitored according to ultraviolet absorbance value at 280 nm. An appropriate amount of 1 M Tris-HCl (pH=9.0) was added to the collection tube containing the elution peak to adjust the pH of the target protein solution to approximately 7.0 to 8.0. A diagram showing protein A affinity chromatography purification of the LIC11 fusion protein is illustrated in FIG. 2. An ultraviolet absorption peak exceeding 1000 mAU can be observed, indicating that the target protein was eluted by the elution buffer.
6) The Protein A column was rinsed with 150 mM NaOH solution, followed by sequential rinse with 10 column volumes of double-distilled water and 20% ethanol, leaving the column filler fully immersed in ethanol.
7) The purity of each elution tube was detected by SDS-PAGE. All elution tubes were combined, and after ultrafiltration and buffer exchange operations, the protein was dissolved in PBS for later use in the next step. The SDS-PAGE electrophoretogram of the fusion protein LIC11 after affinity chromatography purification is illustrated in FIG. 3. The target protein was obtained after affinity chromatography. The molecular weight of the sample subjected to non-reducing treatment was between 100 kDa and 150 kDa, while the molecular weight of the single chain of the sample subjected to reducing treatment was between 50 kDa and 70 kDa, with a single band observed.

### 5. Western Blot Verification of LIC11 Fusion Protein Expression

1) An appropriate amount of the LIC11 fusion protein purified by affinity chromatography was subjected to SDS-PAGE. Electrophoresis conditions were 90 V for 20 min and then 100 V for 70 min.
2) The target protein was transferred to a PVDF membrane using the wet transfer method. The transfer condition was 200 mA for 90 min.
3) After transfer, the PVDF membrane was blocked with 5% skimmed milk at room temperature for 2 h or overnight at 4° C.
4) A goat anti-human heavy chain and light chain-HRP antibody was directly used as the secondary antibody to verify the expression of the antibody fragment in the LIC11 fusion protein. The incubation time was 1 h at room temperature.
5) After membrane washing and ECL chemiluminescence, the results illustrated in FIG. 4 were obtained. These results demonstrated that the non-reduced LIC11 fusion protein exhibited a band between 100 kDa and 150 kDa, while the reduced fusion protein exhibited heavy chain bands between 50 kDa and 70 kDa, which were consistent with the SDS-PAGE results of the previous purified protein. In addition, the binding of the secondary antibody to the antibody fragment of the LIC11 fusion protein in Western Blot confirmed the expression of this portion.
   Example 2: In vitro Enzymatic Cleavage of LIC11 Fusion Protein

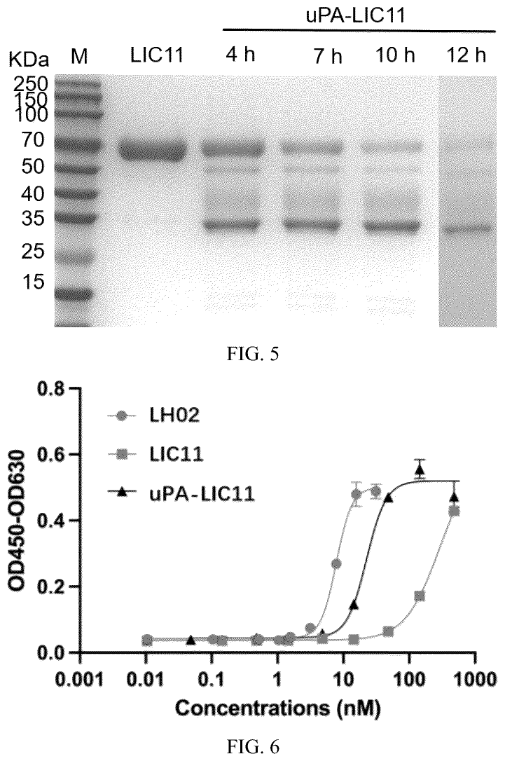

The fusion protein LIC11 prepared in Example 1 and urokinase uPA (0.25 μg/μL) were added to an EP tube at a mass ratio of 20:1, where the mass of LIC11 was 10 μg, and the mass of the urokinase was 0.5 μg. Then, the volume was made up to 20 μL with PBS. Cleavage was performed at 25° C. for 4 h, 7 h, 10 h, and 12 h, respectively, and the cleavage situations at different incubation times were verified by SDS-PAGE. As illustrated in FIG. 5, LIC11 can be cleaved by the urokinase, and the cleavage became more complete as the incubation time increased. The SDS-PAGE results showed that the molecular weight of the uncleaved LIC11 fusion protein was approximately 70 kDa. After cleavage by uPA, a band around 35 kDa was observed, corresponding to the Fc fragment, and a faint band at 40 kDa was observed, corresponding to the IL-15 complex (abbreviated as ILR). The released ILR was a glycosylated protein. Due to the influence of glycosylation, its position in SDS-PAGE was diffused and corresponded to a higher molecular weight than the theoretical one (23.5 kDa), so only a faint band can be observed at around 40 kDa.

### Example 3: Activity Assay of LIC11 Fusion Protein

### Fusion Protein Treatment:

1) 4 μL of uPA (concentration of 0.25 μg/μL) was placed in an EP tube and 20 μg (i.e., 20 μL) of the LIC11 fusion protein prepared in Example 1 was added. The mixture was mixed thoroughly and subjected to cleavage at 37° C. for 12 h.
2) The above-described reaction solution was diluted to 500 μL with RPMI 1640+10% FBS, filtered through a 0.22 μm filter, and then subjected to a three-fold serial dilution. 20 μL of the uncleaved LIC11 fusion protein was diluted to 500 μL with RPMI 1640+10% FBS, filtered through a 0.22 μm filter membrane, and then subjected to a three-fold serial dilution to serve as the control group. Additionally, the positive control LH02 protein was prepared using the same method as in Example 1. Its amino acid sequence was set forth in SEQ ID NO: 9, and its encoding nucleotide sequence was set forth in SEQ ID NO: 10. The IL-15 positive control LH02 was diluted to 500 μL with RPMI 1640+10% FBS, filtered through a 0.22 μm filter membrane, and then subjected to a 3-fold serial dilution.

### Cell Treatment:

1) Mole cells were cultured in suspension using 1640 complete medium (RPMI 1640, Gibco, USA) containing 10 ng/mL commercial recombinant human granulocyte macrophage colony-stimulating factor (hGM-CSF).
2) When cells were in good condition, they were washed twice with complete medium without hGM-CSF, and the cell density was diluted to 4×10⁵ cells/mL. 50 μL of the cell suspension was added to each well of a 96-well plate (i.e., 20,000 cells per well).

### Proliferation Assay:

1) After starving the cells for 4 h, 50 μL of the above-described gradient-diluted LIC11 cleaved by the urokinase was added to each well. The uncleaved LIC11 was used as the control group.
2) After culturing at 37° C. for 4 days, 10 μL/well of CCK-8 (Cell Counting Kit-8) solution was added. After incubation at 37° C. for 2 hours to 3 hours, the absorbance at a wavelength of 450 nm was measured.
3) Data were subjected to four-parameter fitting using GraphPad Prism 8 software to evaluate the proliferation-promoting effect of the fusion protein on lymphocytes. The results were as illustrated in FIG. 6. The activity of the positive control LH02 in promoting Mole proliferation was 4.50 nM. In contrast, the activity of LIC11 without urokinase cleavage in promoting Mo7e cells proliferation was weak. Even at a concentration of 600 nM, it failed to reach the plateau phase, with an activity reduced by at least 150-fold. However, after cleavage of LIC11, its calculated EC₅₀ was 22.53 nM, and its activity in promoting Mo7e cells proliferation was enhanced by at least 20-fold compared with uncleaved LIC11. These results suggested that the Fc fragment and cytokine complex exerted a masking effect on the activity of the cytokine in the uncleaved state, and the released cytokine complex possessed higher biological activity after the linker was cleaved. As a result, the LIC11 fusion protein exhibited improved safety and biological activity by reducing systemic toxic and side effects while maintaining significant anti-tumor effects.

This experimental example also showed that although both were the fusion protein of Fc and IL-15 superagonists, when the fusion mode of IL-15 and the receptor in the complex was sushi-IL-15 (named RLI) and IL-15 was fused to the C-terminus of Fc, that is, the structure of LH02, the Fc exerted no masking effect on the biological activity of IL-15. Only when IL-15 was fused to the C-terminus of Fc and the N-terminus of the sushi domain can Fc exert a masking effect on the activity of IL-15.

### Example 4: Preparation of LIC15 Fusion Protein

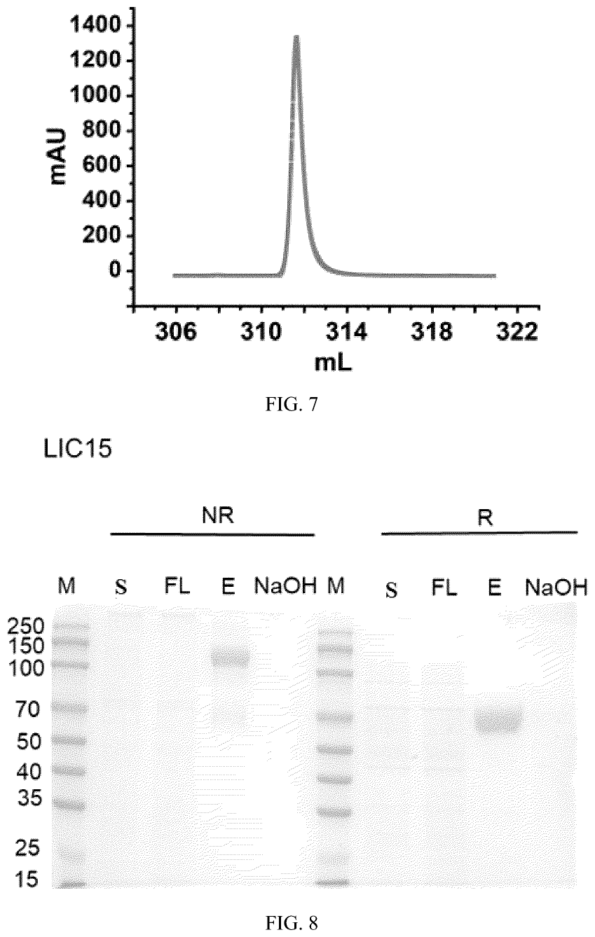

The cleavable linker in LIC11 contained a urokinase substrate sequence. After replacing this sequence with a matrix metalloproteinase substrate sequence, the fusion protein was named LIC15. The structure of LIC15 was similar to that of LIC11, as illustrated in FIG. 1a. The Fc fragment in the LIC15 fusion protein was located at the N-terminus and linked to the IL-15 complex via a cleavable linker, which was capable of being cleaved by MMP enzyme (matrix metalloproteinase). The LIC15 fusion protein was prepared using the same method as in Example 1. The nucleotide sequence prepared by chemical gene synthesis and PCR technology was set forth in SEQ ID NO: 15, and this nucleotide sequence encoded the LIC15 fusion protein with the amino acid sequence as set forth in SEQ ID NO: 16. The elution peak of protein A affinity chromatography and SDS-PAGE are illustrated in FIG. 7 and FIG. 8, respectively. An ultraviolet absorbance peak exceeding 1000 mAU was observed, indicating that the target protein was eluted by the elution buffer. The non-reduced LIC15 fusion protein exhibited a band between 100 kDa and 150 kDa, while the reduced fusion protein exhibited a single band around 70 kDa, corresponding to the single chain of the fusion protein.

### Example 5: Enzymatic Cleavage and Activity Assay of LIC15 Fusion Protein

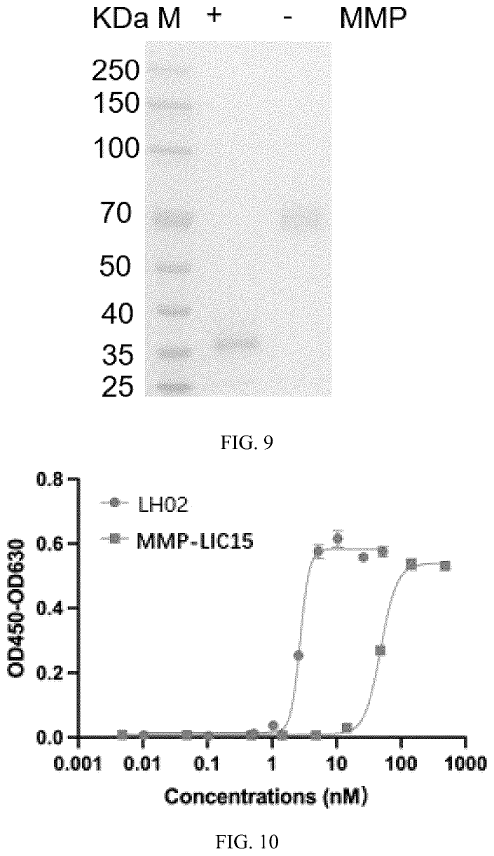

The enzymatic cleavage efficiency of matrix metalloproteinase on the LIC15 fusion protein was tested using a method similar to that in Example 2. 1 μg of activated matrix metalloproteinase rhMMP-2 was added to 100 μg of the LIC15 fusion protein at a mass ratio of 1:100, followed by incubation at 37° C. overnight. Then, the changes in protein bands before and after enzymatic cleavage were detected by SDS-PAGE. The results are illustrated in FIG. 9. The molecular weight of the reduced sample was approximately 70 kDa before enzymatic cleavage. After cleavage by MMP-2, a band around 35 kDa was observed, corresponding to the Fc fragment, and a faint band at 40 kDa was observed, corresponding to the complex ILR of released IL-15 and the sushi domain.

The activity of the LIC15 fusion protein in promoting Mo7e cell proliferation was detected using the same method as in Example 3, with LH02 serving as the positive control. Before enzymatic cleavage, LIC15 at a concentration of 25 μg/mL (equivalent to 448 nM) did not cause significant proliferation of Mo7e cells. The EC₅₀ values of LH02 and LIC15 fusion protein after enzymatic cleavage in promoting Mole cell proliferation were 2.74 nM and 48.56 nM, respectively, as illustrated in FIG. 10. This indicated that the design of LIC15 achieved the masking effect of the Fc fragment in the fusion protein on the activity of IL-15 and can partially restore the activity of IL-15 after enzymatic cleavage. In addition, in in vivo applications, this design can prevent the activation of peripheral immune cells in normal tissues and restore the activity of IL-15 through release in tumor tissues, improving the medication safety and anti-tumor targeting of IL-15.

### Example 6: Preparation of Different Fusion Proteins

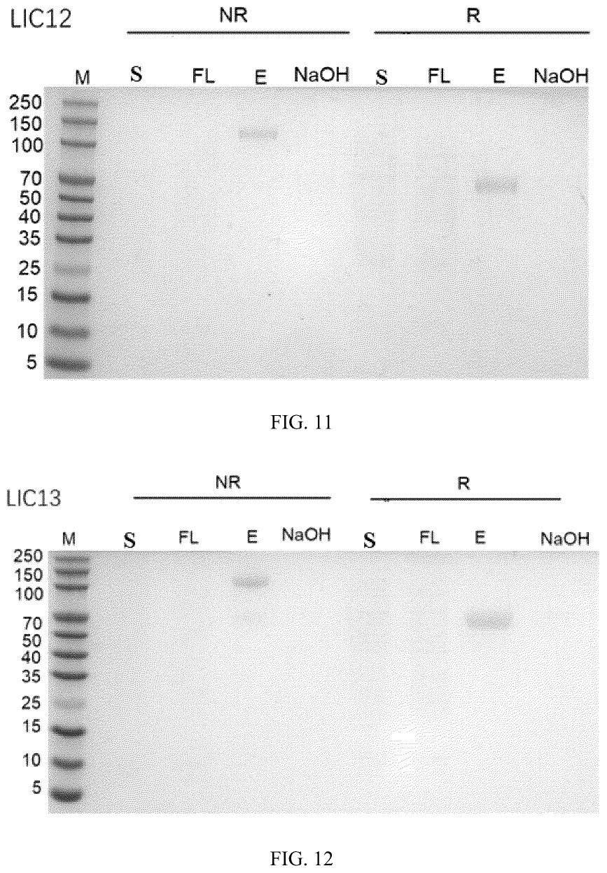

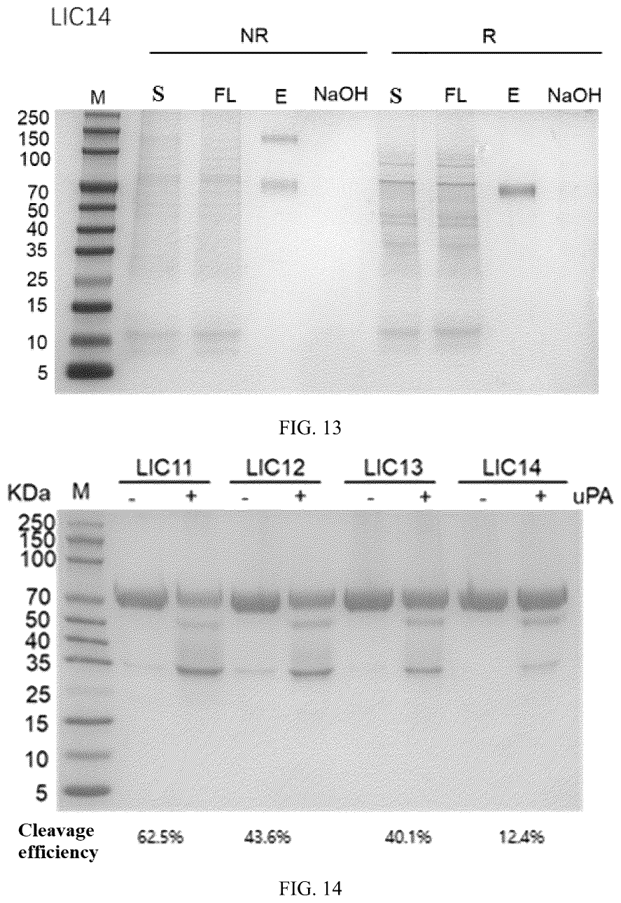

The LIC12, LIC13, and LIC14 fusion proteins were prepared using the same method as in Example 1, with the second structural unit (i.e., the cleavable linker) respectively shortened by 8, 16, or 13 amino acids relative to LIC11. Specifically, their flexible peptide segments between the cleavage substrate sequence and IL-15 were shortened by 8, 8, or 13 amino acids, respectively. In other words, the flexible peptide segments between the enzymatic cleavage substrate sequence and IL-15 in these fusion proteins were 5, 5, and 0 amino acids in length, respectively, and the flexible peptide segment between the cleavage substrate sequence and IL-15 in LIC11 was 13 amino acids in length. The amino acid sequences of the cleavable linkers used in the LIC11, LIC12, LIC13, and LIC14 fusion proteins were set forth in SEQ ID NO: 29, SEQ ID NO: 30, SEQ ID NO: 31, and SEQ ID NO: 32, respectively. The nucleotide sequences prepared by chemical gene synthesis and PCR technology were set forth in SEQ ID NO: 17, SEQ ID NO: 18, and SEQ ID NO: 19, respectively. These nucleotide sequences encoded the amino acid sequences of the LIC12, LIC13, and LIC14 fusion proteins, which were set forth in SEQ ID NO: 20, SEQ ID NO: 21, and SEQ ID NO: 22, respectively. The Protein A affinity chromatography and SDS-PAGE results are illustrated in FIG. 11, FIG. 12, and FIG. 13. The non-reduced LIC12, LIC13, and LIC14 fusion protein exhibited bands at 100 kDa to 150 kDa, while the reduced fusion proteins exhibited bands around 70 kDa, corresponding to the single chain of the fusion proteins.

### Example 7: In Vitro Enzymatic Cleavage and Activity Assays of LIC12, LIC13, and LIC14, as Well as Antitumor Efficacy Studies

1. The enzymatic cleavage efficiency of urokinase on different fusion proteins was detected using the same method as in Example 2, with incubation at 25° C. for 14 h. As illustrated in FIG. 14, the cleavage efficiency of the four fusion proteins with different linkers, detected by SDS-PAGE, was 62.5% for LIC11, 43.6% for LIC12, 40.1% for LIC13, and 12.4% for LIC14. It can be seen that all the different fusion proteins can be cleaved by urokinase. Also, LIC11 exhibited the highest cleavage efficiency, followed by LIC12 and LIC13, while the cleavage efficiency of LIC14 was significantly lower than that of LIC11, LIC12, and LIC13. In addition, LIC14, with a 13-amino acid deletion, exhibited the lowest cleavage efficiency, even lower than that of LIC13, which had a 16-amino acid deletion. A comparison of the two sequences revealed that, between the enzyme cleavage substrate and the IL-15 sequence, LIC13 had a flexible peptide segment for linking, while LIC14 lacked such a flexible peptide segment, indicating that steric hindrance of IL-15 affected substrate recognition by the protease. Therefore, the above-described results demonstrated that the sequence of the second structural unit can significantly influence the cleavage efficiency. Among the components of the second structural unit, the sequence of the flexible peptide segment between the substrate and IL-15 sequence had the greatest impact on the enzymatic cleavage efficiency. When the flexible peptide segment between the enzymatic cleavage substrate sequence and IL-15 were 5 to 13 amino acids in length, the fusion protein exhibited superior enzymatic cleavage efficiency.

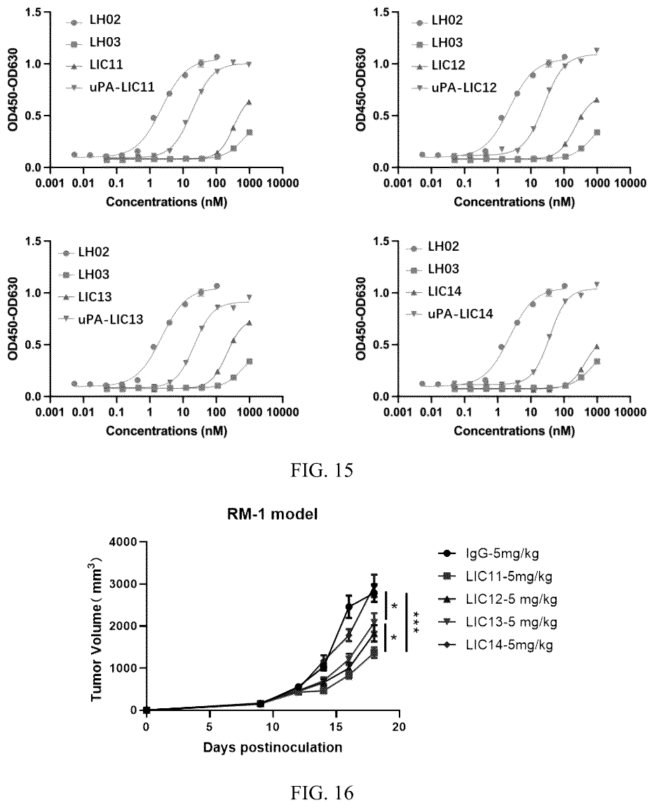

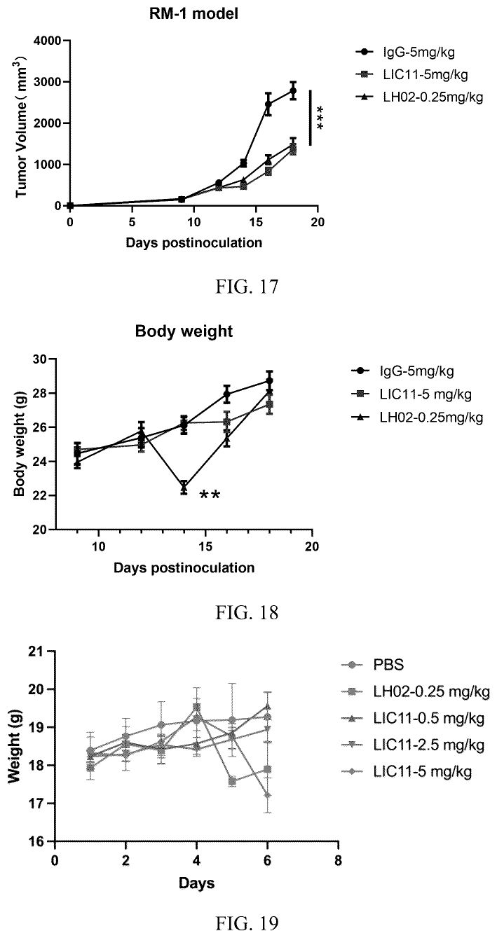

Further, the effects of the above-described fusion proteins on the proliferative activity of Mole before and after cleavage were detected using the same method as in Example 3. As illustrated in FIG. 15, compared with the positive control LH02, LH03 containing a non-cleavable linker (which lacked the uPA substrate sequence compared with LH02 and therefore cannot be cleaved by uPA, and an amino acid sequence of its heavy chain was set forth in SEQ ID NO: 33), and LIC11, LIC12, LIC13, and LIC14 containing a cleavable linker all exhibited significantly reduced effects on the proliferative activity of Mole, with EC₅₀ values of 2.26 nM, 713.3 nM, 331.1 nM, 216.6 nM, 209.3 nM, and 431.8 nM, respectively (in the order of LH02, LH03, LIC11, LIC12, LIC13, and LIC14). After cleaved by uPA, the EC₅₀ values of LIC11, LIC12, LIC13, and LIC14 for Mole proliferative activity were 19.0 nM, 25.0 nM, 22.0 nM, and 37.3 nM, respectively. Based on the above-described data, compared with the positive control LH02, the activities of LIC11, LIC12, LIC13, and LIC14 were reduced by 146-fold, 96-fold, 93-fold, and 191-fold, respectively, while after cleavage, their activities were recovered by 17.4-fold, 8.6-fold, 9.5-fold, and 11.6-fold, respectively. The above-described results suggested that the sequence of the cleavable linker can significantly influence the degree of IL-15 activity being masked in the fusion protein. From comprehensive consideration of factors such as the degree of masking, cleavage efficiency, and restored activity after cleavage, the fusion protein prepared using the cleavable linker of SEQ ID NO: 29 (e.g., LIC11) exhibited the best comprehensive performance among the linkers with four different sequences.
2. Antitumor pharmacodynamic studies were conducted on LIC11 prepared in Example 1, and LIC12, LIC13, and LIC14 prepared in Example 6, with IgG serving as the negative control and LH02 as the positive control. Prostate cancer RM-1 cells were cultured, resuspended in PBS, and then subcutaneously inoculated into C57 mice. Each mouse was inoculated with 5×10⁵ cells in a volume of 100 μL. Intravenous administration was conducted on the 9th, 12th, and 15th days after inoculation, with the negative control IgG administered at 5 mg/kg, the positive control LH02 at 0.25 mg/kg, and each of the LIC11 to LIC14 fusion proteins at 5 mg/kg. The mouse tumor growth curve (FIG. 16 and FIG. 17) and body weights (FIG. 18) were recorded. It was observed that, LIC11 exhibited the best tumor-inhibiting effect, LIC12 and LIC13 showed comparable anti-tumor effects, while the tumor growth curve of LIC14 was close to that of IgG. In addition, LH02 induced a significant decrease in the body weight of mice, but LIC11 did not, indicating that the prodrug molecule LIC11 had good safety. This experiment confirmed that the fusion protein LIC11 capable of cleavage and releasing active IL-15 can improve safety (compared with LH02) and had significant anti-tumor effects due to the targeted release of IL-15 activity in tumor tissues. Furthermore, the data from the four groups of fusion proteins showed that cleavage efficiency was crucial for the tumor-inhibiting effect. That is, LIC11 with high enzymatic cleavage efficiency exhibited the best anti-tumor effect, while LIC14 with lower enzymatic cleavage efficiency had no significant anti-tumor effect.

### Example 8: Safety Evaluation of LIC11 Fusion Protein

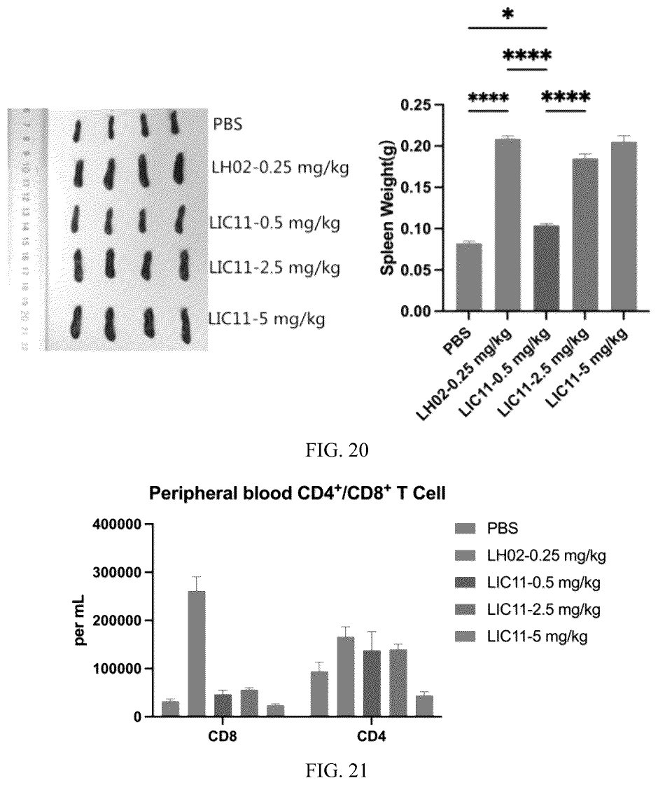

Results of the in vitro cell proliferation assay in Example 3 showed that the activity of IL-15 in the structure of LIC11 prepared in Example 1 was effectively masked, suggesting that LIC11 had low peripheral immune stimulatory activity and systemic toxic and side effects. Balb/c mice aged 6 to 8 weeks were used to evaluate the safety of LIC11. Female Balb/c mice were intravenously injected with PBS, LH02 (0.25 mg/kg), or LIC11 (0.5, 2.5, or 5 mg/kg) on day 1 and day 4. Then, the mice were weighed and their survival states were observed. Experimental results showed that LH02, administered twice at a dose of 0.25 mg/kg, induced severe weight loss, slowed movement, and frizzy hair in the mice. In contrast, LIC11 significantly induced weight loss in mice only at a dose at 5 mg/kg (FIG. 19). Six days later, the mice were sacrificed, and their spleens were isolated, observed, and weighed. The results are illustrated in FIG. 20. LH02 significantly stimulated splenomegaly at a dose of 0.25 mg/kg, while LIC11 fusion protein caused no obvious splenomegaly at a dose of 0.25 mg/kg, exhibited significantly lower stimulatory effect on the spleen than that of LH02 at a dose of 2.5 mg/kg, and exhibited comparable stimulatory effect on the spleen to that of LH02 at a dose of 5 mg/kg. These results demonstrated that compared with the positive control LH02, LIC11 exhibited significantly improved safety in mice due to its masking effect on IL-15 activity.

To further explore the intrinsic mechanism underlying the improved safety of LIC11, orbital blood collection was performed on the mice after the above-described administration. Subsequently, lymphocytes were then isolated from the peripheral blood using lymphocyte separation medium from Dakewe Company, and the changes in CD8⁺ T cells and NK cells in the peripheral blood were analyzed by flow cytometry. The specific procedures were as follows:
1) Extraction of Blood Lymphocytes: 0.3 mL of freshly collected mouse anticoagulated blood was diluted with RPMI 1640 at a 1:1 ratio. 3 mL of lymphocyte separation medium was added to a 15 mL centrifuge tube. The diluted blood was carefully layered on top of the lymphocyte separation medium to prevent mixing the interface of the two liquids. Centrifugation was performed at 800 g with a horizontal rotor for 20 minutes at room temperature by setting the acceleration and deceleration to the third gear. Subsequent steps were the same as those for the separation of mouse splenic lymphocytes.
2) Red blood cell lysis: The single-cell suspension obtained in the previous step was centrifuged at 3000 rpm for 5 min. The supernatant was discarded, and the collected lymphocytes were added to 0.5 mL of red blood cell lysis buffer, gently pipetted to mix thoroughly, and lysed at room temperature for 2 min. Centrifugation was performed at 400 to 500 g for 5 min at 4° C. and the red supernatant was discarded.
3) Flow cytometry: Cells were suspended in PBS+0.5% FBS and then two samples were prepared for detection of NK cells and CD8⁺ T cells. Anti-CD16/32 was added and incubated at 4° C. in the dark for 15 min. The cell surface markers were stained as follows: NK cells (anti-CD45.2-PE, anti-CD3-FITC, anti-Nkp46-Alexa Flour647); and CD8⁺ T cells (anti-CD45.2-PE, anti-CD3-FITC, anti-CD8-APC). After incubating at 4° C. in the dark for 30 min, cells were washed with PBS+0.5% FBS and detected on a flow cytometer.

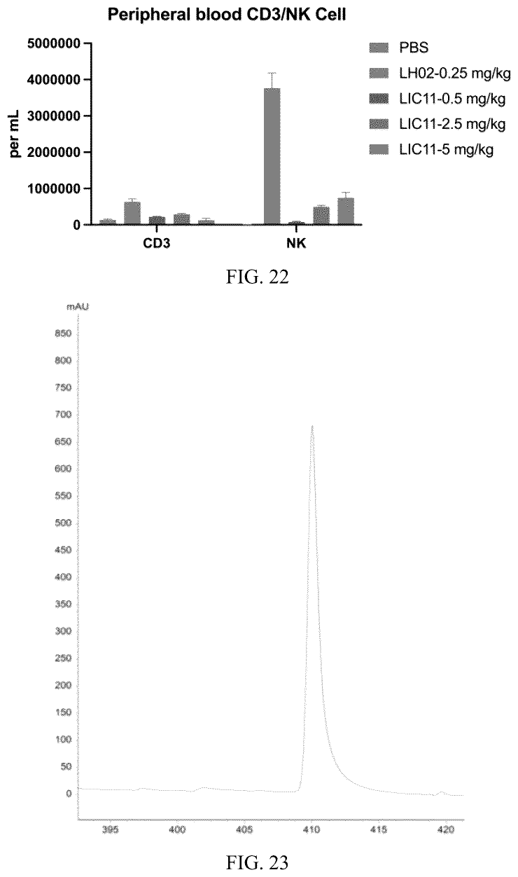

The detection results are illustrated in FIG. 21 and FIG. 22. Compared with the PBS group, LH02 significantly stimulated the proliferation of CD8⁺ T cells and NK cells in peripheral blood. In contrast, at three doses, the effects of LIC11 on promoting proliferation of CD8⁺ T cells and NK cells in peripheral blood were both significantly weaker than those of LH02. The above-described results suggested that the safety of LIC11 was significantly superior to that of LH02, which also confirmed the innovation and practicality of the present disclosure.

### Example 9: Preparation of LIC110 Fusion Protein

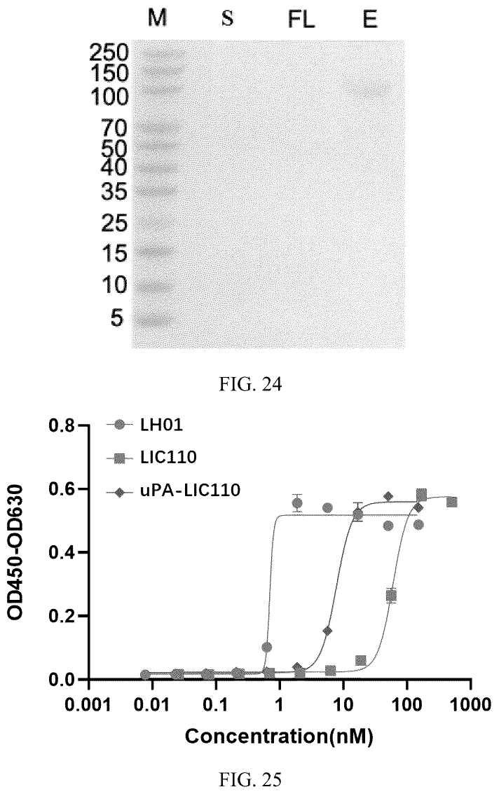

In this example, Fab of the PD-L1 antibody was used as the first structural unit. To the C-terminus of its heavy chain, a cleavable linker and a complex of IL-15 and sushi were fused. The resulting fusion protein was named LIC110, with a structure illustrated in FIG. 1c. The LIC110 fusion protein was prepared using the same method as in Example 1. The nucleotide sequence of the LIC110 heavy chain prepared by chemical gene synthesis and PCR technology was set forth in SEQ ID NO: 23. This nucleotide sequence encoded the heavy chain of the LIC110 fusion protein (SEQ ID NO: 24). The nucleotide and amino acid sequences of the LIC110 light chain were identical to those of the anti-PD-L1 light chain (whose amino acid and nucleotide sequences were set forth in SEQ ID NO: 2 and SEQ ID NO: 7, respectively). The elution peak of protein L affinity chromatography and SDS-PAGE are illustrated in FIG. 23 and FIG. 24, respectively. An ultraviolet absorbance peak exceeding 650 mAU was observed, indicating that the target protein was eluted by the elution buffer. The LIC110 fusion protein without non-reducing treatment exhibited a single band around 100 kDa.

Example 10: In vitro Enzymatic Cleavage and Activity Assay of LIC110

The cleavable linker in LIC110 prepared in Example 9 contained a urokinase substrate. The same method as in Example 3 was used to detect the difference in its effect on proliferative activity of Mo7e before and after cleavage by urokinase. As illustrated in FIG. 25, compared with the positive control LH02, the effect of LIC110 on proliferative activity of Mole was significantly reduced, with EC₅₀ values of 0.69 nM and 60.2 nM, respectively, representing an 87-fold decrease in activity. Furthermore, compared with that before cleavage, the activity was restored by 7.7-fold after cleavage. The above-described results suggested that when the first structural unit was the Fab fragment, the activity of the cytokine can be effectively masked and restored after enzymatic cleavage and release.

### Example 11: Preparation of LIC18 Fusion Protein

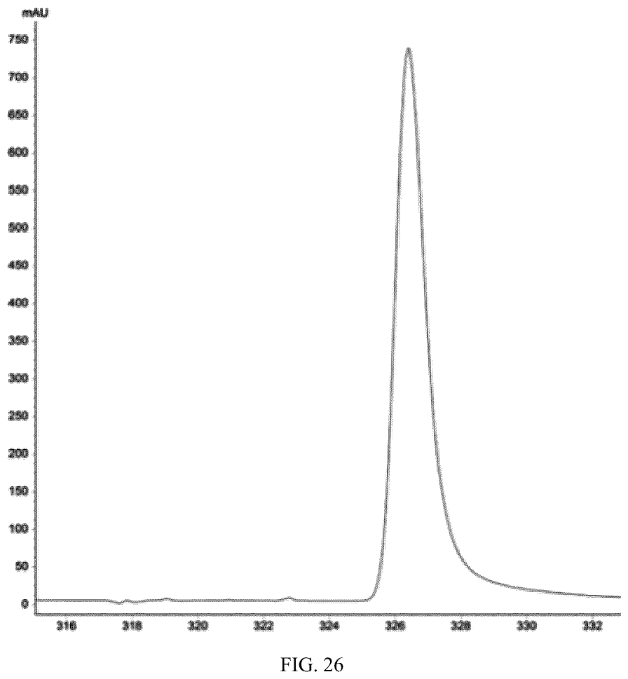

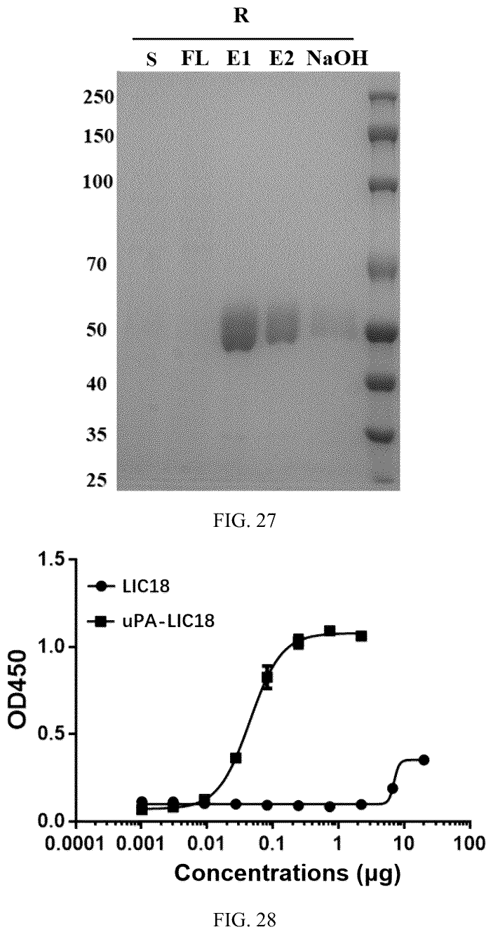

The LIC18 fusion protein was prepared using the same method as in Example 1. The nucleotide sequence prepared by chemical gene synthesis and PCR technology was set forth in SEQ ID No: 12. This nucleotide sequence encoded the LIC18 fusion protein with the amino acid sequence as set forth in SEQ ID No: 11. As illustrated in FIG. 1f, the LIC18 fusion protein included from the N-terminus to the C-terminus: an IL-15Rα fragment containing the sushi domain, IL-15, a cleavable linker containing a urokinase substrate, and an Fc fragment. The elution peak of Protein A affinity chromatography and SDS-PAGE of the prepared fusion protein are illustrated in FIG. 26 and FIG. 27, respectively. An ultraviolet absorbance peak exceeding 700 mAU can be observed, indicating that the target protein was eluted by the elution buffer. The reduced LIC18 fusion protein exhibited a single band at greater than 50 kDa.

### Example 12: Activity Assay of LIC18 Fusion Protein

The changes in the effect of the LIC18 fusion protein prepared in Example 11 on proliferative activity of Mole cells before and after cleavage by urokinase were detected using the same method as in Example 3. As illustrated in FIG. 28, the effect of the uncleaved LIC18 fusion protein on proliferative activity of Mo7e cells was very weak, and did not reach the plateau phase at a concentration of 20 μg/mL. In contrast, the EC₅₀ value of the cleaved LIC18 fusion protein was 0.04494 μg/mL, and its effect on the proliferative activity of Mo7e cells was restored by at least 400-fold.

### Example 13: Preparation of Anti-PD-L1/IL-15 Immunocytokine (LH05) Capable of Cleavage and Release of IL-15

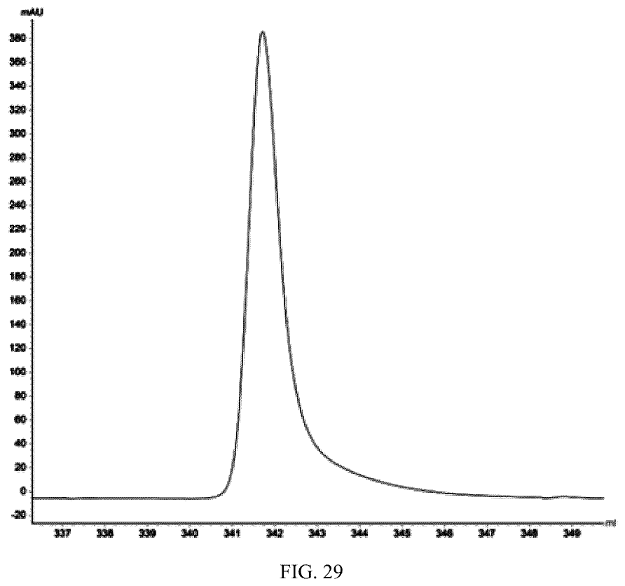

The nucleotide sequences as set forth in SEQ ID No: 7 and SEQ ID No: 8 were prepared by gene synthesis and PCR technology. These nucleotide sequences encoded the LH05 fusion protein (an amino acid sequence of the light chain set forth in SEQ ID No: 2, meaning an amino acid sequence of the light chain of the anti-PD-L1 antibody, and an amino acid sequence of the heavy chain set forth in SEQ ID No: 6). As illustrated in FIG. 1n, the anti-PD-L1 antibody in the LH05 fusion protein was located at the N-terminus, and its C-terminus was fused to IL-15 (with the N72D mutation) and the IL-15Rα-sushi domain fragment via a cleavable linker. In terms of sequence, LH05 was formed by fusing the anti-PD-L1 antibody Fab to the N-terminus of LIC11. The LH05 fusion protein was prepared using the same method as in Example 1. A diagram showing protein A affinity chromatography purification is illustrated in FIG. 29. An ultraviolet absorption peak exceeding 350 mAU can be observed, indicating that the target protein was eluted by the elution buffer. The SDS-PAGE electrophoretogram is illustrated in FIG. 30. The target protein was obtained after affinity chromatography. The molecular weight of the non-reduced sample was greater than 250 kDa. The molecular weight of the heavy chain of the reduced sample was between 80 kDa and 100 kDa, and the molecular weight of the light chain was approximately 25 kDa, with a single band.

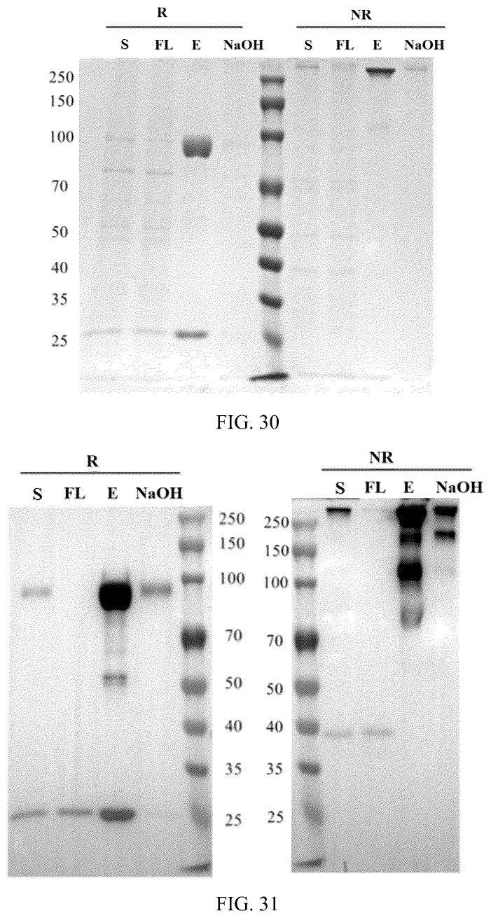

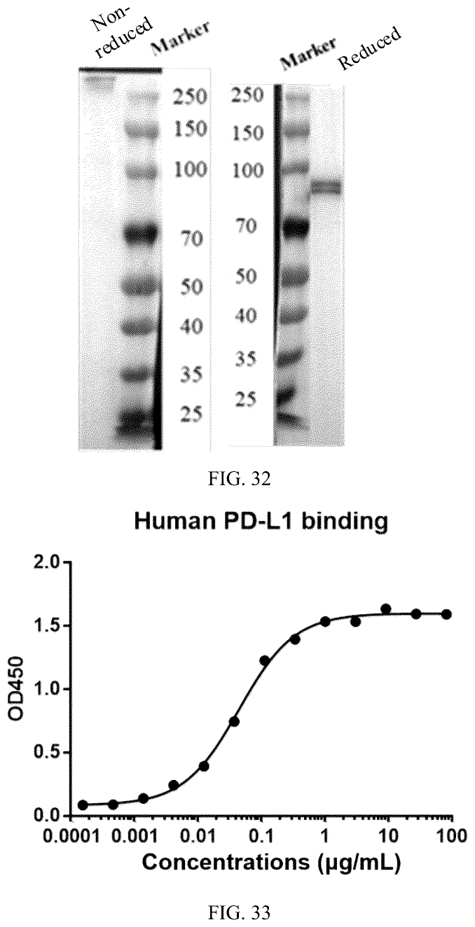

The Western blot experiment was performed using the same method as in Example 1 to verify the expression of the LH05 fusion protein. The same sample was transferred to two PVDF membranes. One membrane was incubated with an anti-human heavy chain and light chain-HRP antibody, which was directly used as the secondary antibody to verify the expression of the antibody fragment in the LH05 fusion protein, and another membrane was incubated with mouse anti-human IL-15 antibody as the primary antibody and goat anti-mouse-HRP antibody as the secondary antibody to verify the expression of IL-15 in the LH05 fusion protein. After antibody incubation, membrane washing, and ECL chemiluminescence, the results were obtained, as illustrated in FIG. 31 and FIG. 32. Both antibody incubation methods confirmed that the non-reduced LH05 fusion protein exhibited a band at greater than 250 kDa, while the molecular weight of the heavy chain of the reduced fusion protein was between 80 kDa and 100 kDa, and the molecular weight of the light chain was approximately 25 kDa. These results were consistent with SDS-PAGE results obtained from the previous protein purification. Binding of antibodies in the Western blot confirmed the expression of both the antibody fragment (FIG. 31) and IL-15 (FIG. 32).

### Example 14: Affinity Detection of LH05 Fusion Protein for Human and Murine PD-L1

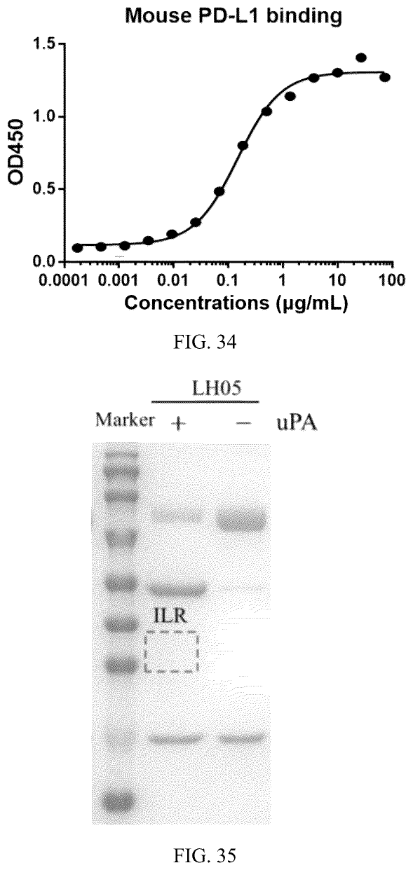

1) Human or mouse PD-L1 antigen was used for coating. PD-L1 was diluted to 1.0 μg/mL using ELISA coating solution. 100 μL was added to each well of a 96-well plate and coated overnight at 4° C.
2) For blocking, the coating solution was discarded and the plate was washed four times with PBST. 250 μL of blocking solution (5% BSA prepared in PBST) was added to each well and incubated at room temperature for 2 h.
3) For incubation with primary antibody (i.e., incubation with LH05 prepared in Example 13), the blocking solution was discarded and the plate was washed four times with PBST. The serially diluted LH05 fusion protein was added to each well at a volume of 100 μL and incubated at room temperature for 2 h.
4) For incubation with secondary antibody, the primary antibody was discarded and the plate was washed four times with PBST. The HRP-conjugated secondary antibody (goat anti-human IgG(H+L)) was diluted with PBST at a ratio of 1:10,000, added to each well at a volume of 100 μL/well, and incubated at room temperature for 1 h.
5) For color development, TMB substrate solution was added to each well at a volume of 100 μL and incubated at 37° C. in the dark for 5 min. The reaction was terminated by adding 50 μL of stop solution (2M H₂SO₄) to each well. Absorbance was measured at 450 nm. Data were processed using GraphPad 7.0, as illustrated in FIG. 33 and FIG. 34. It can be seen that, the LH05 fusion protein had high affinity for both human and murine PD-L1.
   Example 15: In vitro Enzymatic Cleavage of LH05 Fusion Protein

The enzymatic cleavage efficiency of urokinase on the LH05 fusion protein prepared in Example 13 was detected using the same method as in Example 2. After incubation at 16° C. for 24 h, SDS-PAGE and Western Blot (mouse anti-human IL-15 antibody as the primary antibody and goat anti-mouse HRP antibody as the secondary antibody) were performed to detect the cleavage states. As illustrated in FIG. 35, LH05 was cleaved by urokinase. The Western blot results showed a newly appeared band between 25 kDa and 35 kDa after cleavage, indicating that urokinase can release the complex ILR of IL-15 and IL-15Rα-sushi (theoretical molecular weight of 23.5 kDa).

### Example 16: Activity Assay of LH05 Fusion Protein

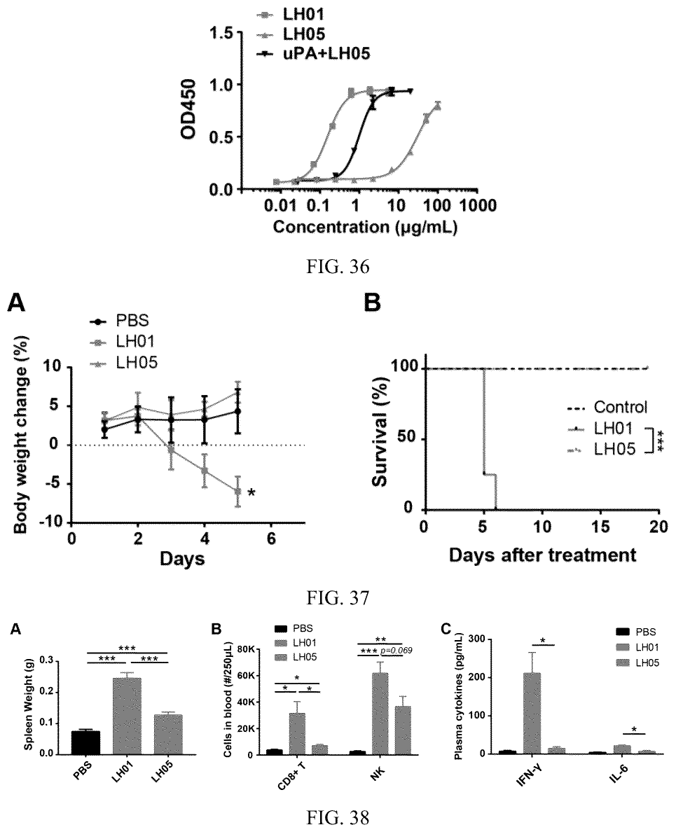

The effect of the LH05 fusion protein prepared in Example 13 on the proliferative activity of Mo7e before and after cleavage was detected using the same method as in Example 3. As a control for the LH05 molecule, the LH01 molecule was prepared, which was an immunocytokine fused with sushi and IL-15 sequentially at the C-terminus of the PD-L1 antibody with high IL-15 activity, and its structure referred to Mol Ther. 2022 Aug. 30; S1525-0016(22)00504-4 (see below). The results are illustrated in FIG. 36. The EC₅₀ values of LH01, LH05, and LH05 after uPA cleavage for promoting Mole cell proliferation were 0.88 nM, 147.5 nM, and 4.9 nM, respectively. These results indicated that when LH05 was not cleaved, the anti-PD-L1 antibody portion of the fusion protein exerted a masking effect on the activity of IL-15. After the linker was cleaved, the released ILR restored its high biological activity. As a result, when the LH05 fusion protein was applied in vivo, it maintained its intact structure in normal tissues, and IL-15 would not activate peripheral immune cells. After reaching tumor tissues, the ILR released by enzymatic cleavage restored its immune activation function and exerted a synergistic anti-tumor effect with the anti-PD-L1 antibody portion. Therefore, compared with LH01, LH05 had better safety and tumor targeting, exerting anti-tumor effects while avoiding systemic toxic and side effects.

### Example 17: Safety Evaluation of LH05 Fusion Protein

Results from the in vitro cell proliferation assay in Example 16 showed that the activity of IL-15 in the LH05 structure was effectively masked, suggesting that LH05 had low peripheral immunostimulatory activity and systemic toxic and side effects. The safety of LH05 was evaluated using the same methods as in Example 8. As illustrated in FIG. 37, LH01 (administered intraperitoneally twice at a dose of 5 mg/kg) induced severe weight loss, slowed movement, and frizzy hair in mice. In contrast, LH05 had no significant effect on body weight at a dose of 10 mg/kg. LH01 induced complete mortality in mice after two doses of 5 mg/kg, while LH05 did not induce mortality after five consecutive doses (once every three days). The above-described results indicated that LH05 exhibited significantly improved safety compared with LH01.

To further explore the intrinsic mechanism underlying the improved safety of LH05, spleens and peripheral blood of mice were collected for analysis. It was found that spleen weights in the LH05 group were significantly higher than those in the PBS group, indicating that immunostimulatory activity of LH05 was not completely masked. In contrast, spleen weights in the LH05 group were significantly lower than those in the LH01 group, indicating that peripheral immunostimulatory activity of LH05 was masked to a large extent (FIG. 38-A). The cell counts of CD8⁺ T cells and NK cells in the peripheral blood showed that LH01 significantly stimulated the proliferation of CD8⁺ T cells and NK cells in the peripheral blood. In contrast, LH05 had a lesser effect on CD8⁺ T cells in the peripheral blood, and although LH05 significantly induced the proliferation of NK cells in the peripheral blood, its effect was significantly weaker than that of LH01 (FIG. 38-B). Compared with the PBS group, LH01 significantly induced an increase in the levels of inflammatory factors (IFN-γ, IL-6) in plasma, while LH05 induced a smaller increase in the levels of plasma inflammatory factors (IFN-γ, IL-6), indicating that LH05 had a lower risk of causing cytokine release syndrome (CRS) (FIG. 38-C).

### Example 18: Anti-Tumor Pharmacodynamics Study of LH05 Fusion Protein

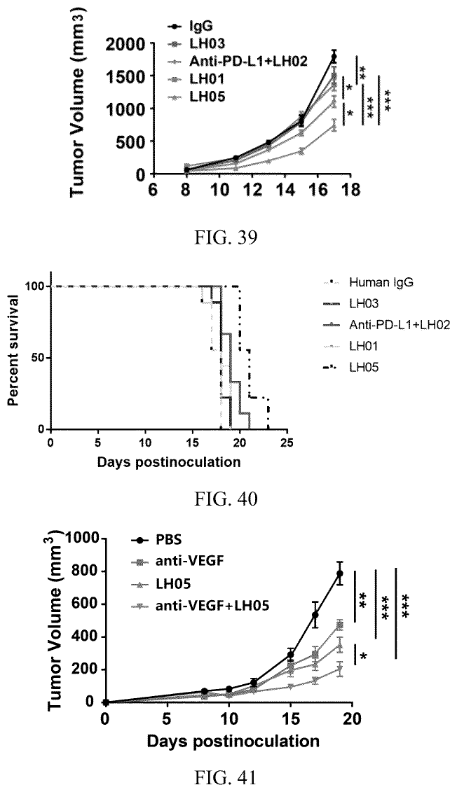

Prostate cancer RM-1 cells were cultured, resuspended in PBS, and subcutaneously inoculated into C57 mice. Each mouse was inoculated with 5×10⁵ cells in a volume of 100 μL. Intravenous administration was conducted on the 9th, 12th, and 15th days after inoculation, with the positive control LH01 administrated at 2.5 mg/kg, the negative control IgG at 10 mg/kg, or LH05 prepared in Example 13 at 10 mg/kg. Furthermore, the non-cleavable form of LH05 (LH03) was administered at 10 mg/kg, along with a combination of the anti-PD-L1 antibody at 10 mg/kg and LH02 at 0.25 mg/kg. The mouse tumor growth curve (FIG. 39) and survival curve (FIG. 40) were recorded. It was observed that, LH05 exhibited significantly superior tumor growth inhibition effect and mouse survival time compared with the negative control, positive control, and the combined treatment group of anti-PD-L1 antibody and IL-15 superagonist LH02, confirming that the fusion protein capable of cleavage and release of active IL-15 exhibited significant anti-tumor effects and good safety.

### Example 19: Anti-tumor Study of Combined Use of LH05 Fusion Protein and Anti-VEGF Monoclonal Antibody

Human HT-29 cells were cultured, resuspended in PBS to a density of 3×10⁷ cells/mL, and then inoculated into immunodeficient NOD-SCID mice. Each mouse was inoculated with 3×10⁶ cells. Subsequently, human PBMCs were injected through the tail vein. The PBMCs were resuspended in PBS to a cell density of 3×10⁷ cells/mL, with a viability exceeding 90%, and each mouse was injected with 3×10⁶ cells per 100 μL. One week after tumor bearing, the mice were administered with 10 mg/kg of the anti-VEGF monoclonal antibody (Bevacizumab Injection, Qilu Pharmaceutical) or the LH05 fusion protein prepared in Example 13, and the negative control group was injected with PBS, intravenously once every three days. Tumor growth situations of the mice were continuously observed and recorded, and the tumor growth curve was plotted as illustrated in FIG. 41. It can be seen from the figure that, the tumor growth inhibition (TGI) rate of the anti-VEGF monotherapy group was 40.6%, the TGI of the LH05 monotherapy group was 53.6%, and the TGI of the combined administration group was 74.5%, indicating that the combination of the two agents exhibited the optimal pharmacodynamic effect, and there was a statistical difference in tumor volume between the combined group and the monotherapy groups. Calculation was conducted using SynergyFinder software, and the combination index (CI) was 1.03. The value greater than 1 indicated that the combined use of LH05 and the anti-VEGF monoclonal antibody exerted a synergistic anti-tumor effect.

### Example 20: Preparation of Anti-EGFR/IL-15 Immunocytokine (LIC23) Capable of Cleavage and Release of IL-15

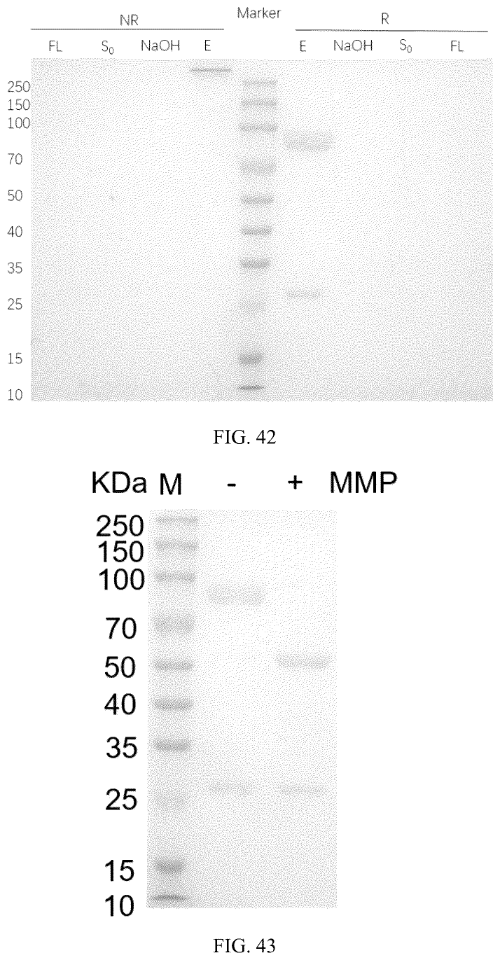

The variable region of the antibody targeting PD-L1 in LH05 prepared in Example 13 was replaced with the variable region of the antibody targeting EGFR and LIC11 in the above-described LH05 was replaced with the sequence of the LIC15 fusion protein from Example 5 to prepare the LIC23 fusion protein using the same method as in Example 1, as illustrated in FIG. 1n. In the heavy chain (or first peptide segment) of the LIC23 fusion protein, the heavy chain of the EGFR antibody was at the N-terminus, and its C-terminus was sequentially fused with IL-15 and the IL-15Rα-sushi fragment via a cleavable linker containing a matrix metalloproteinase. That is, LIC23 had a heavy chain with an amino acid sequence as set forth in SEQ ID No: 25 and a light chain (or referred to as the second peptide segment) with an amino acid sequence as set forth in SEQ ID No: 26. In terms of sequence, LIC23 was formed by fusing an anti-EGFR antibody Fab to the N-terminus of LIC15. The prepared LIC23 fusion protein was subjected to SDS-PAGE electrophoresis detection, as illustrated in FIG. 42. The target protein was obtained after affinity chromatography. The molecular weight of the heavy chain of the reduced sample was between 80 kDa and 100 kDa, and the molecular weight of the light chain was approximately 25 kDa, with a single band.

### Example 21: Enzymatic Cleavage Assay of LIC23 Fusion Protein

The enzymatic cleavage efficiency of the LIC23 fusion protein prepared in Example 20 was tested using a method similar to that used in Example 2. 1 μg of activated matrix metalloproteinase rhMMP-2 was added to 100 μg of the LIC23 fusion protein (at a mass ratio of 1:100) and incubated overnight at 37° C. The changes in protein bands before and after enzymatic cleavage were detected by SDS-PAGE, as illustrated in FIG. 43. The molecular weight of the heavy chain was between 80 kDa and 100 kDa before enzymatic cleavage, and this molecular weight decreased to around 50 kDa after enzymatic cleavage, indicating that MMP-2 released the complex ILR of IL-15 and IL-15Rα-sushi from the heavy chain (theoretical molecular weight of 23.5 kDa). The released ILR exhibited heterogeneous molecular weights due to glycosylation, resulting in diffused bands. Furthermore, the total amount of the protein after cleavage was low, making it difficult to observe clearly in FIG. 43.

### Example 22: Activity Assay of LIC23 Fusion Protein

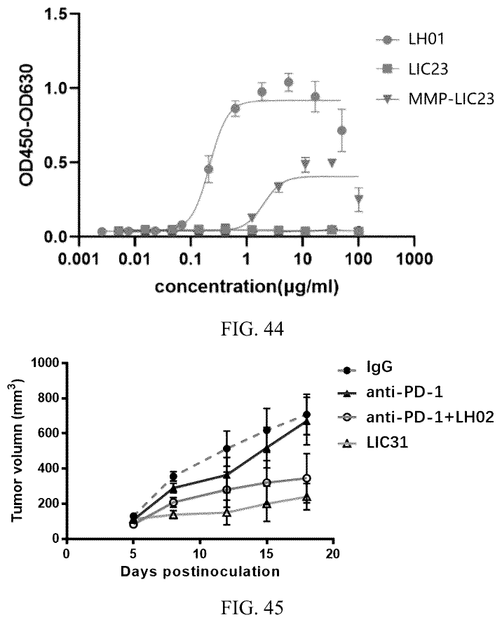

The effect of the LIC23 fusion protein prepared in Example 20 on proliferative activity of Mo7e before and after cleavage was detected using the same method as in Example 3. The LH01 molecule in Example 6 was used as the positive control for IL-15 activity. The results are illustrated in FIG. 44. The EC₅₀ value of LH01 for promoting Mole cell proliferation was 1.06 nM. LIC23 exhibited no proliferative activity of Mole at a concentration of 100 μg/mL (equivalent to 486.6 nM). However, after cleavage by MMP, the EC₅₀ restored to 125.39 nM. These results indicated that when LIC23 was not cleaved, the anti-EGFR antibody portion of the fusion protein exerted a masking effect on the activity of IL-15. When the linker was cleaved, the released ILR restored its high biological activity. As a result, when the LH23 fusion protein was applied in vivo, it maintained its intact structure in normal tissues, and IL-15 would not activate peripheral immune cells. After reaching tumor tissues, the ILR released by enzymatic cleavage restored its immune activation function and exerted a synergistic anti-tumor effect with the anti-EGFR antibody. Therefore, compared with anti-EGFR antibody and IL-15 superagonist, LIC23 had better safety and tumor targeting, exerting anti-tumor effects while avoiding systemic toxic and side effects.

### Example 23: Preparation and Anti-Tumor Studies of Anti-PD-1/IL-15 Immunocytokine (LIC31) Capable of Cleavage and Release of IL-15

The variable region of the antibody targeting EGFR in LIC23 prepared in Example 20 was replaced with the variable region of the antibody targeting PD-1 to prepare the LIC31 fusion protein using the same method as in Example 1. As illustrated in FIG. 1n, the PD-1 antibody in the LIC31 fusion protein was located at the N-terminus, and its C-terminus was sequentially fused with IL-15 and the IL-15Rα-sushi fragment via a cleavable linker containing the matrix metalloproteinase. In terms of sequence, LIC31 was formed by fusing the anti-PD-1 antibody Fab to the N-terminus of LIC15, and LIC31 had a heavy chain (i.e., the first peptide segment) with an amino acid sequence as set forth in SEQ ID No: 27 and a light chain (or referred to as the second peptide segment) with an amino acid sequence as set forth in SEQ ID No: 28.

Animal experiments were conducted to verify that the masking and release of IL-15 activity can improve the safety and anti-tumor efficacy of LIC31 compared with the PD-1 antibody and LH02. In the immunodeficient NCG mouse model, human glioma U87 cells were inoculated and resuspended in PBS to a density of 2.5×10⁷ cells/mL, and each mouse was inoculated with a volume of 100 μL. Four days later, human PBMCs were inoculated via the tail vein at a dose of 4×10⁶ cells/100 μL per mouse. On day 5, the mice were randomly divided into four groups (n=6), which were injected with human immunoglobulin IgG (negative control), anti-PD-1 monoclonal antibody, anti-PD-1 monoclonal antibody combined with the IL-15 superagonist LH02, or LIC31. The administration dose was 10 mg/kg for the antibody and immunoglobulin, and 1 mg/kg for the IL-15 superagonist. Intraperitoneal administration was performed once every three days. The length and width of tumors were measured using a vernier scale, and the tumor volume of each group was calculated. During the experiment, mice in the combined use group of anti-PD-1 monoclonal antibody and IL-15 superagonist LH02 were observed to exhibit weight loss and lethargy, reflecting the toxic and side effects of LH02 at a dose of 1 mg/kg. FIG. 45 showed the tumor growth curve, from which it can be observed that the tumor-inhibiting effect of the LIC31 fusion protein was significantly superior to that of the anti-PD-1 monoclonal antibody treatment group or the combined use group of anti-PD-1 monoclonal antibody and IL-15 superagonist LH02. This demonstrated that the fusion protein capable of cleavage and release of active IL-15 can improve safety, allowing the dose to be increased to 10 mg/kg, and exhibited better anti-tumor effects compared with the combined use group of anti-PD-1 monoclonal antibody and the IL-15 superagonist LH02.

### Example 24: Preparation of LIC20 Fusion Protein

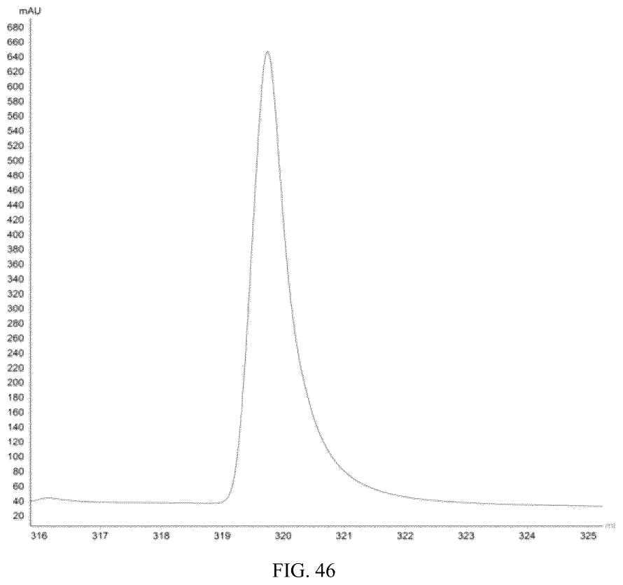

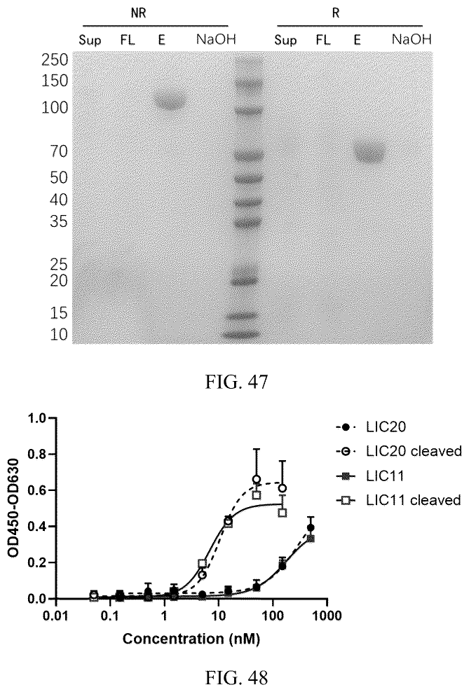

The LIC20 fusion protein with an amino acid sequence as set forth in SEQ ID No: 39 was prepared using the same method as in Example 1. Its structure is illustrated in FIG. 1a. The amino acid sequences of the first and third structural units are identical to those of LIC11. The length of the cleavable linker of the second structural unit is the same as that of LIC11, and its amino acid sequence includes the AAN sequence, as set forth in SEQ ID No: 35. The elution peak of Protein A affinity chromatography and SDS-PAGE of the prepared fusion protein are illustrated in FIG. 46 and FIG. 47, respectively. An ultraviolet absorbance peak exceeding 700 mAU can be observed, indicating that the target protein was eluted by the elution buffer. The reduced LIC20 fusion protein exhibited a single band at greater than 50 kDa.

### Example 25: Activity Assay of LIC20 Fusion Protein

The changes in the effect of the LIC20 fusion protein prepared in Example 24 on proliferative activity of Mole cells before and after cleavage by urokinase were detected using the same method as in Example 3, with LIC11 from Example 1 used as a control. As illustrated in FIG. 48, the effect of LIC20 on proliferative activity of Mole cells, before or after cleavage, was comparable to that of LIC11, with EC₅₀ values of 190 nM and 175.15 nM before cleavage, and 8.6 nM and 6.84 nM after cleavage by uPA, respectively. These data indicate that the fusion protein LIC20, using the AAN sequence as a flexible linker in the second structural unit, exhibited comparable cleavage efficiency, IL-15 activity masking efficiency, and post-cleavage activity levels as the LIC11 control.

### Example 26: Preparation and Activity Assay of LIC19 Fusion Protein

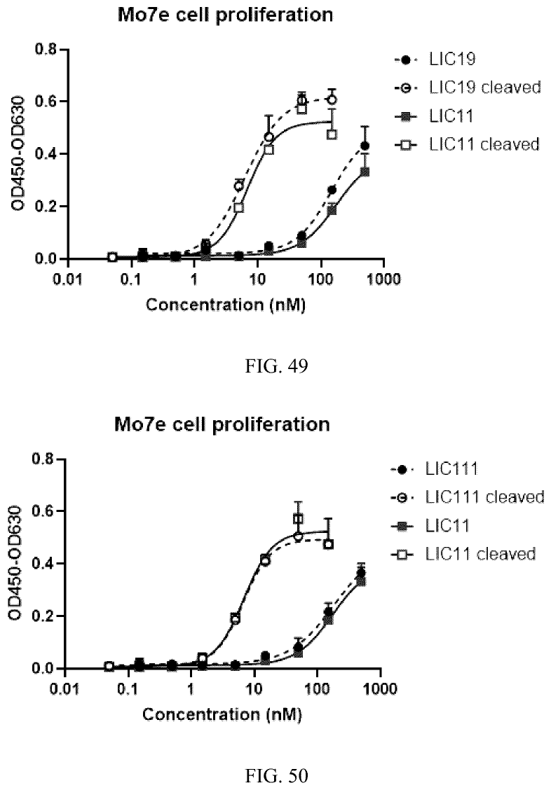

The LIC19 fusion protein was prepared using the same method as in Example 1, with the light chain amino acid sequence as set forth in SEQ ID No: 40, and the heavy chain amino acid sequence as set forth in SEQ ID No: 41. Its structure is illustrated in FIG. 1b. The first structural unit is the Fab sequence of LIC23, and the amino acid sequences of the second and third structural units are identical to those of LIC11. After purification of LIC19 by Protein A affinity chromatography, the changes in the effect of the LIC19 fusion protein on proliferative activity of Mole cells before and after cleavage by urokinase were detected using the same method as in Example 3, with LIC11 from Example 1 used as a control. As illustrated in FIG. 49, the effect of LIC19 on proliferative activity of Mole cells, before or after cleavage, was comparable to that of LIC11, with EC₅₀ values of 146.80 nM and 175.15 nM before cleavage, and 6.23 nM and 6.84 nM after cleavage by uPA, respectively. These data indicate that compared with the LIC11 control, the fusion protein LIC19, using Fab as the first structural unit, also effectively masked IL-15 activity, responded to enzymatic cleavage, and restored IL-15 activity after cleavage.

### Example 27: Preparation and Activity Assay of LIC111 Fusion Protein

The LIC111 fusion protein was prepared using the same method as in Example 1, with its C-terminus fused with a scFv targeting PD-L1. The amino acid sequence is as set forth in SEQ ID No: 42, and the structure is illustrated in FIG. 1e. The first structural unit is scFv-Fc fusion protein, and the amino acid sequences of the second structural unit and the third structural unit are identical to those of LIC11. After purification of LIC111 by Protein A affinity chromatography, the changes in the effect of the LIC19 fusion protein on proliferative activity of Mole cells before and after cleavage by urokinase were detected using the same method as in Example 3, with LIC11 from Example 1 used as a control. As illustrated in FIG. 50, the effect of LIC111 on proliferative activity of Mole cells, before or after cleavage, was comparable to that of LIC11, with EC₅₀ values of 181.50 nM and 175.15 nM before cleavage, and 6.70 nM and 6.84 nM after cleavage by uPA, respectively. These data indicate that compared with the LIC11 control, the fusion protein LIC111, using scFv-Fc fusion protein as the first structural unit, also effectively masked IL-15 activity and restored IL-15 activity after cleavage.

In summary, in specific embodiments, when the first structural unit of the present disclosure includes other antibody or antigen-binding fragment thereof targeting a tumor antigen or an immune checkpoint, which can be selected from, for example, Claudin 18.2, GPC3, LAG3, TIM-3, CTLA-4, etc., the fusion protein will exhibit cytokine activity masking and tissue-targeted release effects similar to those of the above-described fusion proteins and will not be enumerated one by one in the present disclosure.

The specific amino acid sequences and nucleotide sequences of the present disclosure are as follows:

Amino acid sequence of human IL-15 (SEQ ID No: 1):
NWVNVISDLKKIEDLIQSMHIDATLYTESDVHPSCKVTAMKCFLLELQVISLESGDASIHDTVEN
LIILANNSLSSNGNVTESGCKECEELEEKNIKEFLQSFVHIVQMFINTS
Amino acid sequence of light chain of anti-PD-L1 antibody
(or referred to as light chain of LH05 fusion protein)
(SEQ ID No: 2):
DIQMTQSPSSLSASVGDRVTITCRASQDVSTAVAWYQQKPGKAPKLLIYSASFLYSGVPSRFSGS
GSGTDFTLTISSLQPEDFATYYCQQYLYHPATFGQGTKVEIKRTVAAPSVFIFPPSDEQLKSGTA
SVVCLLNNFYPREAKVQWKVDNALQSGNSQESVTEQDSKDSTYSLSSTLTLSKADYEKHKVYACE
VTHQGLSSPVTKSFNRGEC
Amino acid sequence of heavy chain of PD-L1 monoclonal antibody
(SEQ ID No: 3):
EVQLVESGGGLVQPGGSLRLSCAASGFTFSDSWIHWVRQAPGKGLEWVAWISPYGGSTYYADSVK
GRFTISADTSKNTAYLQMNSLRAEDTAVYYCARRHWPGGFDYWGQGTLVTVSSASTKGPSVFPLA
PSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYASTYRVVSVLTVLHQDWLNGKEYKCKVS
NKALPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK
Amino acid sequence of human IL-15N72D mutant (SEQ ID No: 4):
NWVNVISDLKKIEDLIQSMHIDATLYTESDVHPSCKVTAMKCFLLELQVISLESGDASIHDTVEN
LIILANDSLSSNGNVTESGCKECEELEEKNIKEFLQSFVHIVQMFINTS
Amino acid sequence of sushi domain of human IL-15Rα
(SEQ ID No: 5):
ITCPPPMSVEHADIWVKSYSLYSRERYICNSGFKRKAGTSSLTECVLNKATNVAHWTTPSLKCIR
DPALVHQRPAPPSTVTTAGV
Amino acid sequence of heavy chain of LH05 fusion protein
(SEQ ID No: 6):
EVQLVESGGGLVQPGGSLRLSCAASGFTFSDSWIHWVRQAPGKGLEWVAWISPYGGSTYYADSVK
GRFTISADTSKNTAYLQMNSLRAEDTAVYYCARRHWPGGFDYWGQGTLVTVSSASTKGPSVFPLA
PSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYASTYRVVSVLTVLHQDWLNGKEYKCKVS
NKALPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGKGSSGGSG
GSGGSGLSGRSDNHGSSGGSGGSGGSGNWVNVISDLKKIEDLIQSMHIDATLYTESDVHPSCKVT
AMKCFLLELQVISLESGDASIHDTVENLIILANDSLSSNGNVTESGCKECEELEEKNIKEFLQSF
VHIVQMFINTSSGGSGGGGSGGGSGGGGSLQITCPPPMSVEHADIWVKSYSLYSRERYICNSGFK
RKAGTSSLTECVLNKATNVAHWTTPSLKCIRDPALVHQRPAPPSTVTTAGV
Nucleotide sequence of light chain of anti-PD-L1 antibody
(or referred to as light chain of LH05 fusion protein)
(SEQ ID No: 7):
Gacatccagatgacccagtctccatcctccctgtctgcatctgtaggagacagagtcaccatcac
ttgccgggcaagtcaggacgtgagcaccgccgtggcttggtatcagcagaaaccagggaaagccc
ctaagctcctgatctatagcgcatccttcttgtatagtggggtcccatcaaggttcagtggcagt
ggatctgggacagatttcactctcaccatcagcagtctgcaacctgaagattttgcaacttacta
ctgtcaacagtacctgtatcacccggccacgttcggccaagggaccaaggtggaaatcaaacgaa
ctgtggctgcaccatctgtcttcatcttcccgccatctgatgagcagttgaaatctggaactgcc
tctgttgtgtgcctgctgaataacttctatcccagagaggccaaagtacagtggaaggtggataa
cgccctccaatcgggtaactcccaggagagtgtcacagagcaggacagcaaggacagcacctaca
gcctcagcagcaccctgacgctgagcaaagcagactacgagaaacacaaagtttacgcctgcgaa
gtcacccatcagggcctgagctcgcccgtcacaaagagcttcaacaggggagagtgt
Nucleotide sequence encoding heavy chain of LH05 fusion protein
(SEQ ID No: 8):
gaggttcaacttgttgaaagtggaggtggactggttcaaccaggaggctctttgagattgtcatg
cgcagctagtggattcactttctcagacagctggatccattgggtgagacaagcaccaggaaagg
gacttgagtgggttgcttggatctccccctacggaggcagcacctactatgctgatagtgttaag
ggaagattcactatttcagccgataccagcaagaatactgcttaccttcagatgaactcattgag
ggcagaagatacagcagtgtactattgcgctagacggcattggcctgggggatttgattattggg
gacaaggaacattggttactgtttctagtgctagcaccaagggcccatcggtcttccccctggca
ccctcctccaagagcacctctgggggcacagcggccctgggctgcctggtcaaggactacttccc
cgaaccggtgaccgtgtcgtggaactcaggcgccctgaccagcggcgtgcacaccttccctgctg
tcctacagtcctcaggactctactccctcagcagcgtggtgaccgtgccctccagcagcttgggc
acccagacctacatctgcaacgtgaatcacaagcccagcaacaccaaggtggacaagaaagtgga
gcccaaatcttgtgacaaaactcacacatgcccaccgtgcccagcacctgaactcctggggggac
catcagtcttcctcttccccccaaaacccaaggacaccctcatgatctcccggacccctgaggtc
acatgcgtggtggtggacgtgagccacgaagaccctgaggtcaagttcaactggtacgtggacgg
cgtggaggtgcataatgccaagacaaagccgcgggaggagcagtacgccagcacgtaccgtgtgg
tcagcgtcctcaccgtcctgcaccaggactggctgaatggcaaggagtacaagtgcaaggtctcc
aacaaagccctcccagcccccatcgagaaaaccatctccaaagccaaagggcagccccgagaacc
acaggtgtacaccctgcccccatcccgggaagagatgaccaagaaccaggtcagcctgacctgcc
tggtcaaaggcttctatcccagcgacatcgccgtggagtgggagagcaatgggcagccggagaac
aactacaagaccacgcctcccgtgctggactccgacggctccttcttcctctacagcaagctcac
cgtggacaagagcaggtggcagcaggggaacgtcttctcatgctccgtgatgcatgaggctctgc
acaaccactacacgcagaagagcctctccctgtctccaggtaaaggaagctctggaggctctgga
ggctctggcggatctggactgagcggcagatctgataatcatggatctagcggcggatctggcgg
atctggaggctccggaaactgggtgaacgtgatctcggacctgaagaagatcgaggacctcatcc
agtcgatgcacatcgacgcgacgctgtacacggagtcggacgtccacccgtcgtgcaaggtcacg
gcgatgaagtgcttcctcctggagctccaagtcatctcgctcgagtcgggggacgcgtcgatcca
cgacacggtggagaacctgatcatcctggcgaacgactcgctgtcgtcgaacgggaacgtcacgg
agtcgggctgcaaggagtgcgaggagctggaggagaagaacatcaaggagttcctgcagtcgttc
gtgcacatcgtccagatgttcatcaacacgtcgagcggaggatctggcggaggaggctctggagg
aggatctggaggcggaggaagcctgcagatcacgtgcccgccccccatgtccgtggagcacgcag
acatctgggtcaagagctacagcttgtactcccgggagcggtacatctgcaactcgggtttcaag
cggaaggccggcacgtccagcctgacggagtgcgtgttgaacaaggccacgaatgtcgcccactg
gacgaccccctcgctcaagtgcatccgcgacccggccctggttcaccagcggcccgcgccaccct
ccaccgtaacaacagcgggagtgtga
Amino acid sequence of LH02 fusion protein (SEQ ID No: 9):
APELLGGPSVFLFPPKPKDTLMISRTPEVTCVVVDVSHEDPEVKFNWYVDGVEVHNAKTKPREEQ
YASTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSC
SVMHEALHNHYTQKSLSLSPGKITCPPPMSVEHADIWVKSYSLYSRERYICNSGFKRKAGTSSLT
ECVLNKATNVAHWTTPSLKCIRDPALVHQRPAPPSTVTTAGVSGGSGGGGSGGGSGGGGSLQNWV
NVISDLKKIEDLIQSMHIDATLYTESDVHPSCKVTAMKCFLLELQVISLESGDASIHDTVENLII
LANDSLSSNGNVTESGCKECEELEEKNIKEFLQSFVHIVQMFINTS
Nucleotide sequence of LH02 fusion protein (SEQ ID No: 10):
gcacctgaactcctggggggaccatcagtcttcctcttccccccaaaacccaaggacaccctcat
gatctcccggacccctgaggtcacatgcgtggtggtggacgtgagccacgaagaccctgaggtca
agttcaactggtacgtggacggcgtggaggtgcataatgccaagacaaagccgcgggaggagcag
tacgccagcacgtaccgtgtggtcagcgtcctcaccgtcctgcaccaggactggctgaatggcaa
ggagtacaagtgcaaggtctccaacaaagccctcccagcccccatcgagaaaaccatctccaaag
ccaaagggcagccccgagaaccacaggtgtacaccctgcccccatcccgggaagagatgaccaag
aaccaggtcagcctgacctgcctggtcaaaggcttctatcccagcgacatcgccgtggagtggga
gagcaatgggcagccggagaacaactacaagaccacgcctcccgtgctggactccgacggctcct
tcttcctctacagcaagctcaccgtggacaagagcaggtggcagcaggggaacgtcttctcatgc
tccgtgatgcatgaggctctgcacaaccactacacgcagaagagcctctccctgtctccaggtaa
aatcacgtgcccgccccccatgtccgtggagcacgcagacatctgggtcaagagctacagcttgt
actcccgggagcggtacatctgcaactcgggtttcaagcggaaggccggcacgtccagcctgacg
gagtgcgtgttgaacaaggccacgaatgtcgcccactggacgaccccctcgctcaagtgcatccg
cgacccggccctggttcaccagcggcccgcgccaccctccaccgtaacaacagcgggagtgagcg
gaggatctggcggaggaggctctggaggaggatctggaggcggaggaagcctgcagaactgggtg
aacgtgatctcggacctgaagaagatcgaggacctcatccagtcgatgcacatcgacgcgacgct
gtacacggagtcggacgtccacccgtcgtgcaaggtcacggcgatgaagtgcttcctcctggagc
tccaagtcatctcgctcgagtcgggggacgcgtcgatccacgacacggtggagaacctgatcatc
ctggcgaacgactcgctgtcgtcgaacgggaacgtcacggagtcgggctgcaaggagtgcgagga
gctggaggagaagaacatcaaggagttcctgcagtcgttcgtgcacatcgtccagatgttcatca
acacgtcg
Amino acid sequence of LIC18 fusion protein (SEQ ID No: 11):
ITCPPPMSVEHADIWVKSYSLYSRERYICNSGFKRKAGTSSLTECVLNKATNVAHWTTPSLKCIR
DPALVHQRPAPPSTVTTAGVSGGSGGGGSGGGSGGGGSLQNWVNVISDLKKIEDLIQSMHIDATL
YTESDVHPSCKVTAMKCFLLELQVISLESGDASIHDTVENLIILANNSLSSNGNVTESGCKECEE
LEEKNIKEFLQSFVHIVQMFINTSGSSGGSGGSGGSGLSGRSDNHGSSGGSGGSGGSGESKYGPP
CPPCPAPEFLGGPSVFLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
Nucleotide sequence of LIC18 fusion protein (SEQ ID No: 12):
Atcacgtgcccgccccccatgtccgtggagcacgcagacatctgggtcaagagctacagcttgta
ctcccgggagcggtacatctgcaactcgggtttcaagcggaaggccggcacgtccagcctgacgg
agtgcgtgttgaacaaggccacgaatgtcgcccactggacgaccccctcgctcaagtgcatccgc
gacccggccctggttcaccagcggcccgcgccaccctccaccgtaacaacagcgggagtgagcgg
aggatctggcggaggaggctctggaggaggatctggaggcggaggaagcctgcagaactgggtga
acgtgatctcggacctgaagaagatcgaggacctcatccagtcgatgcacatcgacgcgacgctg
tacacggagtcggacgtccacccgtcgtgcaaggtcacggcgatgaagtgcttcctcctggagct
ccaagtcatctcgctcgagtcgggggacgcgtcgatccacgacacggtggagaacctgatcatcc
tggcgaacaactcgctgtcgtcgaacgggaacgtcacggagtcgggctgcaaggagtgcgaggag
ctggaggagaagaacatcaaggagttcctgcagtcgttcgtgcacatcgtccagatgttcatcaa
cacgtcgggaagctctggaggctctggaggctctggcggatctggactgagcggcagatctgata
atcatggatctagcggcggatctggcggatctggaggctccggagagtccaaatatggtccccca
tgcccaccatgcccagcacctgagttcctggggggaccatcagtcttcctgttccccccaaaacc
caaggacactctcatgatctcccggacccctgaggtcacgtgcgtggtggtggacgtgagccagg
aagaccccgaggtccagttcaactggtacgtggatggcgtggaggtgcataatgccaagacaaag
ccgcgggaggagcagttcaacagcacgtaccgtgtggtcagcgtcctcaccgtcctgcaccagga
ctggctgaacggcaaggagtacaagtgcaaggtctccaacaaaggcctcccgtcctccatcgaga
aaaccatctccaaagccaaagggcagccccgagagccacaggtgtacaccctgcccccatcccag
gaggagatgaccaagaaccaggtcagcctgacctgcctggtcaaaggcttctaccccagcgacat
cgccgtggagtgggagagcaatgggcagccggagaacaactacaagaccacgcctcccgtgctgg
actccgacggctccttcttcctctacagcaggctaaccgtggacaagagcaggtggcaggagggg
aatgtcttctcatgctccgtgatgcatgaggctctgcacaaccactacacacagaagagcctctc
cctgtctctgggtaaa
Nucleotide sequence of LIC11 fusion protein (SEQ ID No: 13):
gagtccaaatatggtcccccatgcccaccatgcccagcacctgagttcctggggggaccatcagt
cttcctgttccccccaaaacccaaggacactctcatgatctcccggacccctgaggtcacgtgcg
tggtggtggacgtgagccaggaagaccccgaggtccagttcaactggtacgtggatggcgtggag
gtgcataatgccaagacaaagccgcgggaggagcagttcaacagcacgtaccgtgtggtcagcgt
cctcaccgtcctgcaccaggactggctgaacggcaaggagtacaagtgcaaggtctccaacaaag
gcctcccgtcctccatcgagaaaaccatctccaaagccaaagggcagccccgagagccacaggtg
tacaccctgcccccatcccaggaggagatgaccaagaaccaggtcagcctgacctgcctggtcaa
aggcttctaccccagcgacatcgccgtggagtgggagagcaatgggcagccggagaacaactaca
agaccacgcctcccgtgctggactccgacggctccttcttcctctacagcaggctaaccgtggac
aagagcaggtggcaggaggggaatgtcttctcatgctccgtgatgcatgaggctctgcacaacca
ctacacacagaagagcctctccctgtctctgggtaaaggaagctctggaggctctggaggctctg
gcggatctggactgagcggcagatctgataatcatggatctagcggcggatctggcggatctgga
ggctccggaaactgggtgaacgtgatctcggacctgaagaagatcgaggacctcatccagtcgat
gcacatcgacgcgacgctgtacacggagtcggacgtccacccgtcgtgcaaggtcacggcgatga
agtgcttcctcctggagctccaagtcatctcgctcgagtcgggggacgcgtcgatccacgacacg
gtggagaacctgatcatcctggcgaacgactcgctgtcgtcgaacgggaacgtcacggagtcggg
ctgcaaggagtgcgaggagctggaggagaagaacatcaaggagttcctgcagtcgttcgtgcaca
tcgtccagatgttcatcaacacgtcgagcggaggatctggcggaggaggctctggaggaggatct
ggaggcggaggaagcctgcagatcacgtgcccgccccccatgtccgtggagcacgcagacatctg
ggtcaagagctacagcttgtactcccgggagcggtacatctgcaactcgggtttcaagcggaagg
ccggcacgtccagcctgacggagtgcgtgttgaacaaggccacgaatgtcgcccactggacgacc
ccctcgctcaagtgcatccgcgacccggccctggttcaccagcggcccgcgccaccctccaccgt
aacaacagcgggagtg
Amino acid sequence of LIC11 fusion protein (SEQ ID No: 14):
ESKYGPPCPPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGKGSSGGSGGSGGSGLSGRSDNHGSSGGSGGSG
GSGNWVNVISDLKKIEDLIQSMHIDATLYTESDVHPSCKVTAMKCFLLELQVISLESGDASIHDT
VENLIILANDSLSSNGNVTESGCKECEELEEKNIKEFLQSFVHIVQMFINTSSGGSGGGGSGGGS
GGGGSLQITCPPPMSVEHADIWVKSYSLYSRERYICNSGFKRKAGTSSLTECVLNKATNVAHWTT
PSLKCIRDPALVHQRPAPPSTVTTAGV
Nucleotide sequence of LIC15 fusion protein (SEQ ID No: 15):
gagtccaaatatggtcccccatgcccaccatgcccagcacctgagttcctggggggaccatcagt
cttcctgttccccccaaaacccaaggacactctcatgatctcccggacccctgaggtcacgtgcg
tggtggtggacgtgagccaggaagaccccgaggtccagttcaactggtacgtggatggcgtggag
gtgcataatgccaagacaaagccgcgggaggagcagttcaacagcacgtaccgtgtggtcagcgt
cctcaccgtcctgcaccaggactggctgaacggcaaggagtacaagtgcaaggtctccaacaaag
gcctcccgtcctccatcgagaaaaccatctccaaagccaaagggcagccccgagagccacaggtg
tacaccctgcccccatcccaggaggagatgaccaagaaccaggtcagcctgacctgcctggtcaa
aggcttctaccccagcgacatcgccgtggagtgggagagcaatgggcagccggagaacaactaca
agaccacgcctcccgtgctggactccgacggctccttcttcctctacagcaggctaaccgtggac
aagagcaggtggcaggaggggaatgtcttctcatgctccgtgatgcatgaggctctgcacaacca
ctacacacagaagagcctctccctgtctctgggtaaaggaagctctggaggctctggaggctctg
gcggatctggacagctgctgggcttcctgaccgccggatctagcggcggatctggcggatctgga
ggctccggaaactgggtgaacgtgatctcggacctgaagaagatcgaggacctcatccagtcgat
gcacatcgacgcgacgctgtacacggagtcggacgtccacccgtcgtgcaaggtcacggcgatga
agtgcttcctcctggagctccaagtcatctcgctcgagtcgggggacgcgtcgatccacgacacg
gtggagaacctgatcatcctggcgaacgactcgctgtcgtcgaacgggaacgtcacggagtcggg
ctgcaaggagtgcgaggagctggaggagaagaacatcaaggagttcctgcagtcgttcgtgcaca
tcgtccagatgttcatcaacacgtcgagcggaggatctggcggaggaggctctggaggaggatct
ggaggcggaggaagcctgcagatcacgtgcccgccccccatgtccgtggagcacgcagacatctg
ggtcaagagctacagcttgtactcccgggagcggtacatctgcaactcgggtttcaagcggaagg
ccggcacgtccagcctgacggagtgcgtgttgaacaaggccacgaatgtcgcccactggacgacc
ccctcgctcaagtgcatccgcgacccggccctggttcaccagcggcccgcgccaccctccaccgt
aacaacagcgggagtg
Amino acid sequence of LIC15 fusion protein (SEQ ID No: 16):
ESKYGPPCPPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGKGSSGGSGGSGGSGQLLGFLTAGSSGGSGGSG
GSGNWVNVISDLKKIEDLIQSMHIDATLYTESDVHPSCKVTAMKCFLLELQVISLESGDASIHDT
VENLIILANDSLSSNGNVTESGCKECEELEEKNIKEFLQSFVHIVQMFINTSSGGSGGGGSGGGS
GGGGSLQITCPPPMSVEHADIWVKSYSLYSRERYICNSGFKRKAGTSSLTECVLNKATNVAHWTT
PSLKCIRDPALVHQRPAPPSTVTTAGV
Nucleotide sequence of LIC12 fusion protein (SEQ ID No: 17):
gagtccaaatatggtcccccatgcccaccatgcccagcacctgagttcctggggggaccatcagt
cttcctgttccccccaaaacccaaggacactctcatgatctcccggacccctgaggtcacgtgcg
tggtggtggacgtgagccaggaagaccccgaggtccagttcaactggtacgtggatggcgtggag
gtgcataatgccaagacaaagccgcgggaggagcagttcaacagcacgtaccgtgtggtcagcgt
cctcaccgtcctgcaccaggactggctgaacggcaaggagtacaagtgcaaggtctccaacaaag
gcctcccgtcctccatcgagaaaaccatctccaaagccaaagggcagccccgagagccacaggtg
tacaccctgcccccatcccaggaggagatgaccaagaaccaggtcagcctgacctgcctggtcaa
aggcttctaccccagcgacatcgccgtggagtgggagagcaatgggcagccggagaacaactaca
agaccacgcctcccgtgctggactccgacggctccttcttcctctacagcaggctaaccgtggac
aagagcaggtggcaggaggggaatgtcttctcatgctccgtgatgcatgaggctctgcacaacca
ctacacacagaagagcctctccctgtctctgggtaaaggaagctctggaggctctggaggctctg
gcggatctggactgagcggcagatctgataatcatggatctagcggcggaaactgggtgaacgtg
atctcggacctgaagaagatcgaggacctcatccagtcgatgcacatcgacgcgacgctgtacac
ggagtcggacgtccacccgtcgtgcaaggtcacggcgatgaagtgcttcctcctggagctccaag
tcatctcgctcgagtcgggggacgcgtcgatccacgacacggtggagaacctgatcatcctggcg
aacgactcgctgtcgtcgaacgggaacgtcacggagtcgggctgcaaggagtgcgaggagctgga
ggagaagaacatcaaggagttcctgcagtcgttcgtgcacatcgtccagatgttcatcaacacgt
cgagcggaggatctggcggaggaggctctggaggaggatctggaggcggaggaagcctgcagatc
acgtgcccgccccccatgtccgtggagcacgcagacatctgggtcaagagctacagcttgtactc
ccgggagcggtacatctgcaactcgggtttcaagcggaaggccggcacgtccagcctgacggagt
gcgtgttgaacaaggccacgaatgtcgcccactggacgaccccctcgctcaagtgcatccgcgac
ccggccctggttcaccagcggcccgcgccaccctccaccgtaacaacagcgggagtg
Nucleotide sequence of LIC13 fusion protein (SEQ ID No: 18):
gagtccaaatatggtcccccatgcccaccatgcccagcacctgagttcctggggggaccatcagt
cttcctgttccccccaaaacccaaggacactctcatgatctcccggacccctgaggtcacgtgcg
tggtggtggacgtgagccaggaagaccccgaggtccagttcaactggtacgtggatggcgtggag
gtgcataatgccaagacaaagccgcgggaggagcagttcaacagcacgtaccgtgtggtcagcgt
cctcaccgtcctgcaccaggactggctgaacggcaaggagtacaagtgcaaggtctccaacaaag
gcctcccgtcctccatcgagaaaaccatctccaaagccaaagggcagccccgagagccacaggtg
tacaccctgcccccatcccaggaggagatgaccaagaaccaggtcagcctgacctgcctggtcaa
aggcttctaccccagcgacatcgccgtggagtgggagagcaatgggcagccggagaacaactaca
agaccacgcctcccgtgctggactccgacggctccttcttcctctacagcaggctaaccgtggac
aagagcaggtggcaggaggggaatgtcttctcatgctccgtgatgcatgaggctctgcacaacca
ctacacacagaagagcctctccctgtctctgggtaaaggaagctctggaggcctgagcggcagat
ctgataatcatggatctagcggcggaaactgggtgaacgtgatctcggacctgaagaagatcgag
gacctcatccagtcgatgcacatcgacgcgacgctgtacacggagtcggacgtccacccgtcgtg
caaggtcacggcgatgaagtgcttcctcctggagctccaagtcatctcgctcgagtcgggggacg
cgtcgatccacgacacggtggagaacctgatcatcctggcgaacgactcgctgtcgtcgaacggg
aacgtcacggagtcgggctgcaaggagtgcgaggagctggaggagaagaacatcaaggagttcct
gcagtcgttcgtgcacatcgtccagatgttcatcaacacgtcgagcggaggatctggcggaggag
gctctggaggaggatctggaggcggaggaagcctgcagatcacgtgcccgccccccatgtccgtg
gagcacgcagacatctgggtcaagagctacagcttgtactcccgggagcggtacatctgcaactc
gggtttcaagcggaaggccggcacgtccagcctgacggagtgcgtgttgaacaaggccacgaatg
tcgcccactggacgaccccctcgctcaagtgcatccgcgacccggccctggttcaccagcggccc
gcgccaccctccaccgtaacaacagcgggagtg
Nucleotide sequence of LIC14 fusion protein (SEQ ID No: 19):
gagtccaaatatggtcccccatgcccaccatgcccagcacctgagttcctggggggaccatcagt
cttcctgttccccccaaaacccaaggacactctcatgatctcccggacccctgaggtcacgtgcg
tggtggtggacgtgagccaggaagaccccgaggtccagttcaactggtacgtggatggcgtggag
gtgcataatgccaagacaaagccgcgggaggagcagttcaacagcacgtaccgtgtggtcagcgt
cctcaccgtcctgcaccaggactggctgaacggcaaggagtacaagtgcaaggtctccaacaaag
gcctcccgtcctccatcgagaaaaccatctccaaagccaaagggcagccccgagagccacaggtg
tacaccctgcccccatcccaggaggagatgaccaagaaccaggtcagcctgacctgcctggtcaa
aggcttctaccccagcgacatcgccgtggagtgggagagcaatgggcagccggagaacaactaca
agaccacgcctcccgtgctggactccgacggctccttcttcctctacagcaggctaaccgtggac
aagagcaggtggcaggaggggaatgtcttctcatgctccgtgatgcatgaggctctgcacaacca
ctacacacagaagagcctctccctgtctctgggtaaaggaagctctggaggctctggaggctctg
gcggatctggactgagcggcagatctgataatcataactgggtgaacgtgatctcggacctgaag
aagatcgaggacctcatccagtcgatgcacatcgacgcgacgctgtacacggagtcggacgtcca
cccgtcgtgcaaggtcacggcgatgaagtgcttcctcctggagctccaagtcatctcgctcgagt
cgggggacgcgtcgatccacgacacggtggagaacctgatcatcctggcgaacgactcgctgtcg
tcgaacgggaacgtcacggagtcgggctgcaaggagtgcgaggagctggaggagaagaacatcaa
ggagttcctgcagtcgttcgtgcacatcgtccagatgttcatcaacacgtcgagcggaggatctg
gcggaggaggctctggaggaggatctggaggcggaggaagcctgcagatcacgtgcccgcccccc
atgtccgtggagcacgcagacatctgggtcaagagctacagcttgtactcccgggagcggtacat
ctgcaactcgggtttcaagcggaaggccggcacgtccagcctgacggagtgcgtgttgaacaagg
ccacgaatgtcgcccactggacgaccccctcgctcaagtgcatccgcgacccggccctggttcac
cagcggcccgcgccaccctccaccgtaacaacagcgggagtg
Amino acid sequence of LIC12 fusion protein (SEQ ID No: 20):
ESKYGPPCPPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGKGSSGGSGGSGGSGLSGRSDNHGSSGGNWVNV
ISDLKKIEDLIQSMHIDATLYTESDVHPSCKVTAMKCFLLELQVISLESGDASIHDTVENLIILA
NDSLSSNGNVTESGCKECEELEEKNIKEFLQSFVHIVQMFINTSSGGSGGGGSGGGSGGGGSLQI
TCPPPMSVEHADIWVKSYSLYSRERYICNSGFKRKAGTSSLTECVLNKATNVAHWTTPSLKCIRD
PALVHQRPAPPSTVTTAGV
Amino acid sequence of LIC13 fusion protein (SEQ ID No: 21):
ESKYGPPCPPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGKGSSGGLSGRSDNHGSSGGNWVNVISDLKKIE
DLIQSMHIDATLYTESDVHPSCKVTAMKCFLLELQVISLESGDASIHDTVENLIILANDSLSSNG
NVTESGCKECEELEEKNIKEFLQSFVHIVQMFINTSSGGSGGGGSGGGSGGGGSLQITCPPPMSV
EHADIWVKSYSLYSRERYICNSGFKRKAGTSSLTECVLNKATNVAHWTTPSLKCIRDPALVHQRP
APPSTVTTAGV
Amino acid sequence of LIC14 fusion protein (SEQ ID No: 22):
ESKYGPPCPPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGKGSSGGSGGSGGSGLSGRSDNHNWVNVISDLK
KIEDLIQSMHIDATLYTESDVHPSCKVTAMKCFLLELQVISLESGDASIHDTVENLIILANDSLS
SNGNVTESGCKECEELEEKNIKEFLQSFVHIVQMFINTSSGGSGGGGSGGGSGGGGSLQITCPPP
MSVEHADIWVKSYSLYSRERYICNSGFKRKAGTSSLTECVLNKATNVAHWTTPSLKCIRDPALVH
QRPAPPSTVTTAGV
Nucleotide sequence of heavy chain of LIC110 fusion protein
(SEQ ID No: 23):
gaggttcaacttgttgaaagtggaggtggactggttcaaccaggaggctctttgagattgtcatg
cgcagctagtggattcactttctcagacagctggatccattgggtgagacaagcaccaggaaagg
gacttgagtgggttgcttggatctccccctacggaggcagcacctactatgctgatagtgttaag
ggaagattcactatttcagccgataccagcaagaatactgcttaccttcagatgaactcattgag
ggcagaagatacagcagtgtactattgcgctagacggcattggcctgggggatttgattattggg
gacaaggaacattggttactgtttctagtgctagcaccaagggcccatcggtcttccccctggca
ccctcctccaagagcacctctgggggcacagcggccctgggctgcctggtcaaggactacttccc
cgaaccggtgaccgtgtcgtggaactcaggcgccctgaccagcggcgtgcacaccttccctgctg
tcctacagtcctcaggactctactccctcagcagcgtggtgaccgtgccctccagcagcttgggc
acccagacctacatctgcaacgtgaatcacaagcccagcaacaccaaggtggacaagaaagtggg
aagctctggaggctctggaggctctggcggatctggactgagcggcagatctgataatcatggat
ctagcggcggatctggcggatctggaggctccggaaactgggtgaacgtgatctcggacctgaag
aagatcgaggacctcatccagtcgatgcacatcgacgcgacgctgtacacggagtcggacgtcca
cccgtcgtgcaaggtcacggcgatgaagtgcttcctcctggagctccaagtcatctcgctcgagt
cgggggacgcgtcgatccacgacacggtggagaacctgatcatcctggcgaacgactcgctgtcg
tcgaacgggaacgtcacggagtcgggctgcaaggagtgcgaggagctggaggagaagaacatcaa
ggagttcctgcagtcgttcgtgcacatcgtccagatgttcatcaacacgtcgagcggaggatctg
gcggaggaggctctggaggaggatctggaggcggaggaagcctgcagatcacgtgcccgcccccc
atgtccgtggagcacgcagacatctgggtcaagagctacagcttgtactcccgggagcggtacat
ctgcaactcgggtttcaagcggaaggccggcacgtccagcctgacggagtgcgtgttgaacaagg
ccacgaatgtcgcccactggacgaccccctcgctcaagtgcatccgcgacccggccctggttcac
cagcggcccgcgccaccctccaccgtaacaacagcgggagtggagcccaaatcttgtgacaaaac
tcacacatgcccaccgtgcccagcacctgaactcctggggggaccatcagtcttcctcttccccc
caaaacccaaggacaccctcatgatctcccggacccctgaggtcacatgcgtggtggtggacgtg
agccacgaagaccctgaggtcaagttcaactggtacgtggacggcgtggaggtgcataatgccaa
gacaaagccgcgggaggagcagtacgccagcacgtaccgtgtggtcagcgtcctcaccgtcctgc
accaggactggctgaatggcaaggagtacaagtgcaaggtctccaacaaagccctcccagccccc
atcgagaaaaccatctccaaagccaaagggcagccccgagaaccacaggtgtacaccctgccccc
atcccgggaagagatgaccaagaaccaggtcagcctgacctgcctggtcaaaggcttctatccca
gcgacatcgccgtggagtgggagagcaatgggcagccggagaacaactacaagaccacgcctccc
gtgctggactccgacggctccttcttcctctacagcaagctcaccgtggacaagagcaggtggca
gcaggggaacgtcttctcatgctccgtgatgcatgaggctctgcacaaccactacacgcagaaga
gcctctccctgtctccaggtaaa
Amino acid sequence of heavy chain of LIC110 fusion protein
(SEQ ID No: 24):
EVQLVESGGGLVQPGGSLRLSCAASGFTFSDSWIHWVRQAPGKGLEWVAWISPYGGSTYYADSVK
GRFTISADTSKNTAYLQMNSLRAEDTAVYYCARRHWPGGFDYWGQGTLVTVSSASTKGPSVFPLA
PSSKSTSGGTAALGCLVKDYFPEPVTVSWNSGALTSGVHTFPAVLQSSGLYSLSSVVTVPSSSLG
TQTYICNVNHKPSNTKVDKKVGSSGGSGGSGGSGLSGRSDNHGSSGGSGGSGGSGNWVNVISDLK
KIEDLIQSMHIDATLYTESDVHPSCKVTAMKCFLLELQVISLESGDASIHDTVENLIILANDSLS
SNGNVTESGCKECEELEEKNIKEFLQSFVHIVQMFINTSSGGSGGGGSGGGSGGGGSLQITCPPP
MSVEHADIWVKSYSLYSRERYICNSGFKRKAGTSSLTECVLNKATNVAHWTTPSLKCIRDPALVH
QRPAPPSTVTTAGVEPKSCDKTHTCPPCPAPELLGGPSVFLFPPKPKDTLMISRTPEVTCVVVDV
SHEDPEVKFNWYVDGVEVHNAKTKPREEQYASTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
Amino acid sequence of heavy chain of LIC23 fusion protein
(SEQ ID No: 25):
QVQLKQSGPGLVQPSQSLSITCTVSGFSLTNYGVHWVRQSPGKGLEWLGVIWSGGNTDYNTPFTS
RLSINKDNSKSQVFFKMNSLQSNDTAIYYCARALTYYDYEFAYWGQGTLVTVSAASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGKGSSGGS
GGSGGSGQLLGFLTAGSSGGSGGSGGSGNWVNVISDLKKIEDLIQSMHIDATLYTESDVHPSCKV
TAMKCFLLELQVISLESGDASIHDTVENLIILANDSLSSNGNVTESGCKECEELEEKNIKEFLQS
FVHIVQMFINTSSGGSGGGGSGGGSGGGGSLQITCPPPMSVEHADIWVKSYSLYSRERYICNSGF
KRKAGTSSLTECVLNKATNVAHWTTPSLKCIRDPALVHQRPAPPSTVTTAGV
Amino acid sequence of light chain of LIC23 fusion protein
(i.e., light chain of anti-EGFR) (SEQ ID No: 26):
DILLTQSPVILSVSPGERVSFSCRASQSIGTNIHWYQQRTNGSPRLLIKYASESISGIPSRFSGS
GSGTDFTLSINSVESEDIADYYCQQNNNWPTTFGAGTKLELKRTVAAPSVFIFPPSDEQLKSGTA
SVVCLLNNFYPREAKVQWKVDNALQSGNSQESVTEQDSKDSTYSLSSTLTLSKADYEKHKVYACE
VTHQGLSSPVTKSFNRGEC
Amino acid sequence of heavy chain of LIC31 fusion protein
(SEQ ID No: 27):
QVQLVESGGGVVQPGRSLRLDCKASGITFSNSGMHWVRQAPGKGLEWVAVIWYDGSKRYYADSVK
GRFTISRDNSKNTLFLQMNSLRAEDTAVYYCATNDDYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKDTLMISRTPEVTCVVV
DVSHEDPEVKFNWYVDGVEVHNAKTKPREEQYASTYRVVSVLTVLHQDWLNGKEYKCKVSNKALP
APIEKTISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGKGSSGGSGGSGGS
GQLLGFLTAGSSGGSGGSGGSGNWVNVISDLKKIEDLIQSMHIDATLYTESDVHPSCKVTAMKCF
LLELQVISLESGDASIHDTVENLIILANDSLSSNGNVTESGCKECEELEEKNIKEFLQSFVHIVQ
MFINTSSGGSGGGGSGGGSGGGGSLQITCPPPMSVEHADIWVKSYSLYSRERYICNSGFKRKAGT
SSLTECVLNKATNVAHWTTPSLKCIRDPALVHQRPAPPSTVTTAGV
Amino acid sequence of light chain of LIC31 fusion protein
(i.e., light chain of anti-PD-1) (SEQ ID No: 28):
DVVMTQSPLSLPVTLGQPASISCRSSQSLLDSDGGTYLYWFQQRPGQSPRRLIYLVSTLGSGVPD
RFSGSGSGTDFTLKISRVEAEDVGVYYCMQLTHWPYTFGQGTKLEIKRTVAAPSVFIFPPSDEQL
KSGTASVVCLLNNFYPREAKVQWKVDNALQSGNSQESVTEQDSKDSTYSLSSTLTLSKADYEKHK
VYACEVTHQGLSSPVTKSFNRGEC
Cleavable peptide linker:
GSSGGSGGSGGSGLSGRSDNHGSSGGSGGSGGSG (SEQ ID NO: 29);
GSSGGSGGSGGSGLSGRSDNHGSSGG (SEQ ID NO: 30);
GSSGGLSGRSDNHGSSGG (SEQ ID NO: 31);
GSSGGSGGSGGSGLSGRSDNH(SEQ ID NO: 32);
GSSGGSGGSGAANLSGRSDNHGSSGGSGGSGGSG (SEQ ID NO: 37)
GSSGGSGGSGGSGQLLGFLTAGSSGGSGGSGGSG (SEQ ID No: 38)
Amino acid sequence of heavy chain of LH03 fusion protein
(SEQ ID No: 33):
EVQLVESGGGLVQPGGSLRLSCAASGFTFSDSWIHWVRQAPGKGLEWVAWISPYGGSTYYADSVK
GRFTISADTSKNTAYLQMNSLRAEDTAVYYCARRHWPGGFDYWGQGTLVTVSSASTKGPSVFPLA
PSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYASTYRVVSVLTVLHQDWLNGKEYKCKVS
NKALPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGKSGGGSGG
GGSGGGGSNWVNVISDLKKIEDLIQSMHIDATLYTESDVHPSCKVTAMKCFLLELQVISLESGDA
SIHDTVENLIILANDSLSSNGNVTESGCKECEELEEKNIKEFLQSFVHIVQMFINTSSGGSGGGG
SGGGSGGGGSLQITCPPPMSVEHADIWVKSYSLYSRERYICNSGFKRKAGTSSLTECVLNKATNV
AHWTTPSLKCIRDPALVHQRPAPPSTVTTAGV
Flexible linker:
SGGSGGGGSGGGSGGGGSLQ (SEQ ID No: 34)
Urokinase (uPA) substrate sequence:
LSGRSDNH (SEQ ID No: 35)
Matrix metalloproteinases (MMP) substrate sequence:
QLLGFLTA (SEQ ID No: 36)
Amino acid sequence of LIC20 fusion protein (SEQ ID No: 39):
ESKYGPPCPPCPAPEFLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIEKTISKAKGQPREPQV
YTLPPSQEEMTKNQVSLTCLVKGFYPSDIAVEWESNGQPENNYKTTPPVLDSDGSFFLYSRLTVD
KSRWQEGNVFSCSVMHEALHNHYTQKSLSLSLGKGSSGGSGGSGAANLSGRSDNHGSSGGSGGSG
GSGNWVNVISDLKKIEDLIQSMHIDATLYTESDVHPSCKVTAMKCFLLELQVISLESGDASIHDT
VENLIILANDSLSSNGNVTESGCKECEELEEKNIKEFLQSFVHIVQMFINTSSGGSGGGGSGGGS
GGGGSLQITCPPPMSVEHADIWVKSYSLYSRERYICNSGFKRKAGTSSLTECVLNKATNVAHWTT
PSLKCIRDPALVHQRPAPPSTVTTAGV
Amino acid sequence of light chain of LIC19 fusion protein
(SEQ ID No: 40):
DILLTQSPVILSVSPGERVSFSCRASQSIGTNIHWYQQRINGSPRLLIKYASESISGIPSRFSGS
GSGTDFTLSINSVESEDIADYYCQQNNNWPTTFGAGTKLELKRTVAAPSVFIFPPSDEQLKSGTA
SVVCLLNNFYPREAKVQWKVDNALQSGNSQESVTEQDSKDSTYSLSSTLTLSKADYEKHKVYACE
VTHQGLSSPVTKSFNRGECGSSGGSGGSGGSGLSGRSDNHGSSGGSGGSGGSGNWVNVISDLKKI
EDLIQSMHIDATLYTESDVHPSCKVTAMKCFLLELQVISLESGDASIHDTVENLIILANDSLSSN
GNVTESGCKECEELEEKNIKEFLQSFVHIVQMFINTSSGGSGGGGSGGGSGGGGSLQITCPPPMS
VEHADIWVKSYSLYSRERYICNSGFKRKAGTSSLTECVLNKATNVAHWTTPSLKCIRDPALVHQR
PAPPSTVTTAGV
Amino acid sequence of heavy chain of LIC19 fusion protein
(SEQ ID No: 41):
QVQLKQSGPGLVQPSQSLSITCTVSGFSLTNYGVHWVRQSPGKGLEWLGVIWSGGNTDYNTPFTS
RLSINKDNSKSQVFFKMNSLQSNDTAIYYCARALTYYDYEFAYWGQGTLVTVSAASTKGPSVFPL
APSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTGSSGGSGGSGGSGLSGRSDNHGSSGGSGGSGGS
GNWVNVISDLKKIEDLIQSMHIDATLYTESDVHPSCKVTAMKCFLLELQVISLESGDASIHDTVE
NLIILANDSLSSNGNVTESGCKECEELEEKNIKEFLQSFVHIVQMFINTSSGGSGGGGSGGGSGG
GGSLQITCPPPMSVEHADIWVKSYSLYSRERYICNSGFKRKAGTSSLTECVLNKATNVAHWTTPS
LKCIRDPALVHQRPAPPSTVTTAGV
Amino acid sequence of LIC111 fusion protein (SEQ ID No: 42):
EVQLVESGGGLVQPGGSLRLSCAASGFTFSDSWIHWVRQAPGKGLEWVAWISPYGGSTYYADSVK
GRFTISADTSKNTAYLQMNSLRAEDTAVYYCARRHWPGGFDYWGQGTLVTVSSGGGGSGGGGSGG
GGSDIQMTQSPSSLSASVGDRVTITCRASQDVSTAVAWYQQKPGKAPKLLIYSASFLYSGVPSRF
SGSGSGTDFTLTISSLQPEDFATYYCQQYLYHPATFGQGTKVEIKESKYGPPCPPCPAPEFLGGP
SVFLFPPKPKDTLMISRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQEGNVFSCSVMHEALH
NHYTQKSLSLSLGKGSSGGSGGSGGSGLSGRSDNHGSSGGSGGSGGSGNWVNVISDLKKIEDLIQ
SMHIDATLYTESDVHPSCKVTAMKCFLLELQVISLESGDASIHDTVENLIILANDSLSSNGNVTE
SGCKECEELEEKNIKEFLQSFVHIVQMFINTSSGGSGGGGSGGGSGGGGSLQITCPPPMSVEHAD
IWVKSYSLYSRERYICNSGFKRKAGTSSLTECVLNKATNVAHWTTPSLKCIRDPALVHQRPAPPS
TVTTAGV

In the description of the present specification, reference to the terms such as “an embodiment,” “some embodiments,” “an example,” “a specific example,” or “some examples” means that a particular feature, structure, material, or characteristic described in connection with the embodiment or example is included in at least one embodiment or example of the present disclosure. The illustrative expressions of the above terms as used in this specification do not necessarily refer to the same embodiment or example. Furthermore, the particular features, structures, materials, or characteristics described may be combined in any suitable manner in one or more embodiments or examples. In addition, unless otherwise conflicting, those skilled in the art will appreciate that the different embodiments or examples described in this specification, as well as the features of different embodiments or examples, may be combined or integrated.

Although the embodiments of the present disclosure have been shown and described above, it will be understood by those of ordinary skill in the art that the above embodiments are exemplary only and should not be construed as limiting the scope of the invention. Alterations, substitutions, and variations can be made to the described embodiments without departing from the scope of the present disclosure.

What is claimed is:

1. A cytokine fusion protein, comprising:
a first structural unit, comprising at least one of an Fc fragment, an antibody, or an antigen-binding fragment, wherein the antibody or the antigen-binding fragment targets a tumor antigen or an immune checkpoint;
a second structural unit, comprising a cleavable peptide linker, wherein the cleavable peptide linker comprises a first flexible peptide segment, a protease substrate sequence, and a second flexible peptide segment; and
a third structural unit, comprising cytokine or a complex of cytokine and a receptor thereof,
wherein the first structural unit is linked to the N- or C-terminus of the third structural unit via the second structural unit, the first flexible peptide segment is located between the first structural unit and the protease substrate sequence, and the second flexible peptide segment is located between the protease substrate sequence and the third structural unit.

2. The fusion protein according to claim 1, wherein the protease substrate sequence is recognized and cleaved by a protease overexpressed in a tumor tissue.

3. The fusion protein according to claim 1, wherein the protease substrate sequence is a matrix metalloproteinase substrate sequence, a serine protease substrate sequence, or an asparagine endopeptidase substrate sequence.

4. The fusion protein according to claim 1, wherein an amino acid sequence of the first flexible peptide segment and the second flexible peptide segment is each independently selected from the group consisting of (GS)n, (GGS)n, (GGSG)n, (GSSG)n, (GGGS)n, (GGGGS)n, (GSGGS)n, AAN, and a combination thereof, where n is any integer between 1 and 20.

5. The fusion protein according to claim 1, wherein the second flexible peptide segment has 5 to 13 amino acids in length.

6. The fusion protein according to claim 1, wherein the cleavable peptide linker has an amino acid sequence as set forth in any of SEQ ID No: 29 to SEQ ID No: 31, SEQ ID No: 37, and SEQ ID No: 38.

7. The fusion protein according to claim 1, wherein the complex of cytokine and the receptor thereof is a complex of IL-15 and a receptor thereof with a sushi domain, and wherein the IL-15 is a human IL-15 molecule or a mutant of the human IL-15 molecule.

8. The fusion protein according to claim 7, wherein the complex of IL-15 and the receptor thereof with the sushi domain comprises a fusion protein of IL-15-flexible linker-sushi domain,
wherein the flexible linker has an amino acid sequence as set forth in SEQ ID No: 34, and wherein the sushi domain in the complex of IL-15 and the receptor thereof with the sushi domain has an amino acid sequence as set forth in SEQ ID No: 5.

9. The fusion protein according to claim 7, wherein the human IL-15 molecule has an amino acid sequence as set forth in SEQ ID No: 1, or wherein the mutant of the human IL-15 molecule has an amino acid sequence as set forth in SEQ ID No: 4.

10. The fusion protein according to claim 1, wherein:
the antibody comprises at least one selected from a polyclonal antibody, a full-length monoclonal antibody, a Fab antibody, a Fab′ antibody, a F(ab′)₂ antibody, a Fv antibody, a single-chain antibody, a single-domain antibody, and a minimum recognition unit; or
the antigen-binding fragment comprises at least one selected from a F(ab′)₂ fragment, a Fab′ fragment, a Fab fragment, a F(ab)₂ fragment, a Fv fragment, a scFv fragment, a scFv-Fc fusion protein, a scFv-Fv fusion protein, and a minimum recognition unit.

11. The fusion protein according to claim 1, wherein:
the fusion protein has an amino acid sequence as set forth in any one of SEQ ID No: 14, SEQ ID No: 16, SEQ ID No: 20, SEQ ID No: 21, SEQ ID No: 39, SEQ ID No: 11, and SEQ ID No: 42; or
the fusion protein comprises a first peptide segment with an amino acid sequence as set forth in SEQ ID No: 6 and a second peptide segment with an amino acid sequence as set forth in SEQ ID No: 2; or
the fusion protein comprises a first peptide segment with an amino acid sequence as set forth in SEQ ID No: 24 and a second peptide segment with an amino acid sequence as set forth in SEQ ID No: 2; or
the fusion protein comprises a first peptide segment with an amino acid sequence as set forth in SEQ ID No: 25 and a second peptide segment with an amino acid sequence as set forth in SEQ ID No: 26; or
the fusion protein comprises a first peptide segment with an amino acid sequence as set forth in SEQ ID No: 27 and a second peptide segment with an amino acid sequence as set forth in SEQ ID No: 28, or
the fusion protein comprises a first peptide segment with an amino acid sequence as set forth in SEQ ID No: 41 and a second peptide segment with an amino acid sequence as set forth in SEQ ID No: 40.

12. The fusion protein according to claim 1, wherein the tumor antigen or immune checkpoint comprises at least one of EGFR, VEGF, Claudin 18.2, Nectin-4, GPC-3, PD-L1, PD-1, TIGIT, LAG3, TIM-3, and CTLA-4, or
wherein the antibody or the antigen-binding fragment is from a human IgG1 or IgG4 antibody.

13. A nucleic acid, encoding the fusion protein according to claim 1.

14. An expression vector, comprising the nucleic acid according to claim 13.

15. A recombinant cell, comprising the nucleic acid according to claim 13.

16. A fusion protein complex, comprising the fusion protein according to claim 1.

17. An immunotherapy cell, expressing the fusion protein according to claim 1.

18. A pharmaceutical composition, comprising the fusion protein according to claim 1.

19. A combination agent or kit, comprising:
the fusion protein according to claim 1, as a first active ingredient; and
a monoclonal antibody targeting a tumor or an immune checkpoint and/or a chemotherapy agent, as a second active ingredient,
optionally, the monoclonal antibody targeting the tumor comprises an anti-VEGF antibody, an anti-PD-1/PD-L1 antibody, and/or an anti-Her2 antibody.

20. A method for treating a cancer, an infectious disease, or an autoimmune disease, comprising:
administering to a subject a pharmaceutically acceptable amount of the fusion protein according to claim 1.


SEQUENCE LISTING (42 sequences)

SEQ ID NO: 1 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 114 aa, synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 5 - protein, 85 aa, synthetic construct
ITCPPPMSVEHADIWVKSYSLYSRERYICNSGFKRKAGTSSLTECVLNKATNVAHWTTPSLKCIRDPALVHQRPAPPSTVTTAGV

SEQ ID NO: 6 - protein, 701 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGSGGSGGSGLSGRSDNHGSSGGSGGSGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 7 - DNA, 642 bp, synthetic construct
  source 1..642: mol_type = other DNA
gacatccagatgacccagtctccatcctccctgtctgcatctgtaggagacagagtcaccatcacttgccgggcaagtcaggacgtgagcaccgccgtggcttggtatcagcagaaaccagggaaagcccctaagctcctgatctatagcgcatccttcttgtatagtggggtcccatcaaggttcagtggcagtggatctgggacagatttcactctcaccatcagcagtctgcaacctgaagattttgcaacttactactgtcaacagtacctgtatcacccggccacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtttacgcctgcgaagtcacccatcagggcctgagctcgcccgtcacaaagagcttcaacaggggagagtgt

SEQ ID NO: 8 - DNA, 2106 bp, synthetic construct
  source 1..2106: mol_type = other DNA
gaggttcaacttgttgaaagtggaggtggactggttcaaccaggaggctctttgagattgtcatgcgcagctagtggattcactttctcagacagctggatccattgggtgagacaagcaccaggaaagggacttgagtgggttgcttggatctccccctacggaggcagcacctactatgctgatagtgttaagggaagattcactatttcagccgataccagcaagaatactgcttaccttcagatgaactcattgagggcagaagatacagcagtgtactattgcgctagacggcattggcctgggggatttgattattggggacaaggaacattggttactgtttctagtgctagcaccaagggcccatcggtcttccccctggcaccctcctccaagagcacctctgggggcacagcggccctgggctgcctggtcaaggactacttccccgaaccggtgaccgtgtcgtggaactcaggcgccctgaccagcggcgtgcacaccttccctgctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcctggggggaccatcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccaggtaaaggaagctctggaggctctggaggctctggcggatctggactgagcggcagatctgataatcatggatctagcggcggatctggcggatctggaggctccggaaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacgactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgagcggaggatctggcggaggaggctctggaggaggatctggaggcggaggaagcctgcagatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacaacagcgggagtgtga

SEQ ID NO: 9 - protein, 436 aa, synthetic construct
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKITCPPPMSVEHADIWVKSYSLYSRERYICNSGFKRKAGTSSLTECVLNKATNVAHWTTPSLKCIRDPALVHQRPAPPSTVTTAGVSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 10 - DNA, 1308 bp, synthetic construct
  source 1..1308: mol_type = other DNA
gcacctgaactcctggggggaccatcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccaggtaaaatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacaacagcgggagtgagcggaggatctggcggaggaggctctggaggaggatctggaggcggaggaagcctgcagaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacgactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcg

SEQ ID NO: 11 - protein, 482 aa, synthetic construct
ITCPPPMSVEHADIWVKSYSLYSRERYICNSGFKRKAGTSSLTECVLNKATNVAHWTTPSLKCIRDPALVHQRPAPPSTVTTAGVSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSSGGSGGSGGSGLSGRSDNHGSSGGSGGSGGSGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - DNA, 1446 bp, synthetic construct
  source 1..1446: mol_type = other DNA
atcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacaacagcgggagtgagcggaggatctggcggaggaggctctggaggaggatctggaggcggaggaagcctgcagaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacaactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgggaagctctggaggctctggaggctctggcggatctggactgagcggcagatctgataatcatggatctagcggcggatctggcggatctggaggctccggagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 13 - DNA, 1446 bp, synthetic construct
  source 1..1446: mol_type = other DNA
gagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaggaagctctggaggctctggaggctctggcggatctggactgagcggcagatctgataatcatggatctagcggcggatctggcggatctggaggctccggaaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacgactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgagcggaggatctggcggaggaggctctggaggaggatctggaggcggaggaagcctgcagatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacaacagcgggagtg

SEQ ID NO: 14 - protein, 482 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSSGGSGGSGGSGLSGRSDNHGSSGGSGGSGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 15 - DNA, 1446 bp, synthetic construct
  source 1..1446: mol_type = other DNA
gagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaggaagctctggaggctctggaggctctggcggatctggacagctgctgggcttcctgaccgccggatctagcggcggatctggcggatctggaggctccggaaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacgactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgagcggaggatctggcggaggaggctctggaggaggatctggaggcggaggaagcctgcagatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacaacagcgggagtg

SEQ ID NO: 16 - protein, 482 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSSGGSGGSGGSGQLLGFLTAGSSGGSGGSGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 17 - DNA, 1422 bp, synthetic construct
  source 1..1422: mol_type = other DNA
gagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaggaagctctggaggctctggaggctctggcggatctggactgagcggcagatctgataatcatggatctagcggcggaaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacgactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgagcggaggatctggcggaggaggctctggaggaggatctggaggcggaggaagcctgcagatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacaacagcgggagtg

SEQ ID NO: 18 - DNA, 1398 bp, synthetic construct
  source 1..1398: mol_type = other DNA
gagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaggaagctctggaggcctgagcggcagatctgataatcatggatctagcggcggaaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacgactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgagcggaggatctggcggaggaggctctggaggaggatctggaggcggaggaagcctgcagatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacaacagcgggagtg

SEQ ID NO: 19 - DNA, 1407 bp, synthetic construct
  source 1..1407: mol_type = other DNA
gagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaaggaagctctggaggctctggaggctctggcggatctggactgagcggcagatctgataatcataactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacgactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgagcggaggatctggcggaggaggctctggaggaggatctggaggcggaggaagcctgcagatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacaacagcgggagtg

SEQ ID NO: 20 - protein, 474 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSSGGSGGSGGSGLSGRSDNHGSSGGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 21 - protein, 466 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSSGGLSGRSDNHGSSGGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 22 - protein, 469 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSSGGSGGSGGSGLSGRSDNHNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 23 - DNA, 2103 bp, synthetic construct
  source 1..2103: mol_type = other DNA
gaggttcaacttgttgaaagtggaggtggactggttcaaccaggaggctctttgagattgtcatgcgcagctagtggattcactttctcagacagctggatccattgggtgagacaagcaccaggaaagggacttgagtgggttgcttggatctccccctacggaggcagcacctactatgctgatagtgttaagggaagattcactatttcagccgataccagcaagaatactgcttaccttcagatgaactcattgagggcagaagatacagcagtgtactattgcgctagacggcattggcctgggggatttgattattggggacaaggaacattggttactgtttctagtgctagcaccaagggcccatcggtcttccccctggcaccctcctccaagagcacctctgggggcacagcggccctgggctgcctggtcaaggactacttccccgaaccggtgaccgtgtcgtggaactcaggcgccctgaccagcggcgtgcacaccttccctgctgtcctacagtcctcaggactctactccctcagcagcgtggtgaccgtgccctccagcagcttgggcacccagacctacatctgcaacgtgaatcacaagcccagcaacaccaaggtggacaagaaagtgggaagctctggaggctctggaggctctggcggatctggactgagcggcagatctgataatcatggatctagcggcggatctggcggatctggaggctccggaaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacgactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgagcggaggatctggcggaggaggctctggaggaggatctggaggcggaggaagcctgcagatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacaacagcgggagtggagcccaaatcttgtgacaaaactcacacatgcccaccgtgcccagcacctgaactcctggggggaccatcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccaggtaaa

SEQ ID NO: 24 - protein, 701 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGSSGGSGGSGGSGLSGRSDNHGSSGGSGGSGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 702 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGSGGSGGSGQLLGFLTAGSSGGSGGSGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 26 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 696 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGSGGSGGSGQLLGFLTAGSSGGSGGSGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 28 - protein, 219 aa, synthetic construct
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 34 aa, synthetic construct
GSSGGSGGSGGSGLSGRSDNHGSSGGSGGSGGSG

SEQ ID NO: 30 - protein, 26 aa, synthetic construct
GSSGGSGGSGGSGLSGRSDNHGSSGG

SEQ ID NO: 31 - protein, 18 aa, synthetic construct
GSSGGLSGRSDNHGSSGG

SEQ ID NO: 32 - protein, 21 aa, synthetic construct
GSSGGSGGSGGSGLSGRSDNH

SEQ ID NO: 33 - protein, 682 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGSGGGGSGGGGSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 34 - protein, 20 aa, synthetic construct
SGGSGGGGSGGGSGGGGSLQ

SEQ ID NO: 35 - protein, 8 aa, synthetic construct
LSGRSDNH

SEQ ID NO: 36 - protein, 8 aa, synthetic construct
QLLGFLTA

SEQ ID NO: 37 - protein, 34 aa, synthetic construct
GSSGGSGGSGAANLSGRSDNHGSSGGSGGSGGSG

SEQ ID NO: 38 - protein, 34 aa, synthetic construct
GSSGGSGGSGGSGQLLGFLTAGSSGGSGGSGGSG

SEQ ID NO: 39 - protein, 482 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSSGGSGGSGAANLSGRSDNHGSSGGSGGSGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 40 - protein, 467 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSSGGSGGSGGSGLSGRSDNHGSSGGSGGSGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 41 - protein, 480 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGSSGGSGGSGGSGLSGRSDNHGSSGGSGGSGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV

SEQ ID NO: 42 - protein, 722 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSSGGSGGSGGSGLSGRSDNHGSSGGSGGSGGSGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRDPALVHQRPAPPSTVTTAGV